SEQUENCE LISTING (432 sequences)

SEQ ID NO: 1 - protein, 33 aa, Homo sapiens
  MOD_RES (20)..(20): Ac-Lys
AALHLYSNTLNFQISEVEPKYYADGEDAYAMKR

SEQ ID NO: 2 - protein, 11 aa, Homo sapiens
  MOD_RES (8)..(8): Ac-Lys
RGLGGGGKWKR

SEQ ID NO: 3 - protein, 11 aa, Homo sapiens
  MOD_RES (10)..(10): Ac-Lys
RGLGGGGKWKR

SEQ ID NO: 4 - protein, 20 aa, Homo sapiens
  MOD_RES (6)..(6): Ac-Lys
KLNCYKITLECLPQNVGFYK

SEQ ID NO: 5 - protein, 16 aa, Homo sapiens
  MOD_RES (13)..(13): Ac-Lys
LNQVIFPVSYNDKFYK

SEQ ID NO: 6 - protein, 13 aa, Homo sapiens
  MOD_RES (3)..(3): Ac-Lys
FYKDVLEVGELAK

SEQ ID NO: 7 - protein, 9 aa, Homo sapiens
  MOD_RES (9)..(9): Ac-Lys
RKATQVGEK

SEQ ID NO: 8 - protein, 5 aa, Homo sapiens
  MOD_RES (4)..(4): Ac-Lys
NYYKR

SEQ ID NO: 9 - protein, 22 aa, Homo sapiens
  MOD_RES (4)..(4): Ac-Lys
IAAKFITHAPPGEFNEVFNDVR

SEQ ID NO: 10 - protein, 12 aa, Homo sapiens
  MOD_RES (7)..(7): Ac-Lys
TKIDWNKILSYK

SEQ ID NO: 11 - protein, 12 aa, Homo sapiens
  MOD_RES (10)..(10): Ac-Lys
STLNEIYFGKTK

SEQ ID NO: 12 - protein, 12 aa, Homo sapiens
  MOD_RES (11)..(11): Ac-Lys
NILDSKPTANKK

SEQ ID NO: 13 - protein, 17 aa, Homo sapiens
  MOD_RES (17)..(17): Ac-Lys
KEELMFFLWAPELAPLK

SEQ ID NO: 14 - protein, 19 aa, Homo sapiens
  MOD_RES (8)..(8): Ac-Lys
LLGWIQNKLPQLPITNFSR

SEQ ID NO: 15 - protein, 16 aa, Homo sapiens
  MOD_RES (15)..(15): Ac-Lys
IDINMSGFNETDDLKR

SEQ ID NO: 16 - protein, 12 aa, Homo sapiens
  MOD_RES (5)..(5): Ac-Lys
GFGGKFGVQMDR

SEQ ID NO: 17 - protein, 15 aa, Homo sapiens
  MOD_RES (5)..(5): Ac-Lys
GFGGKYGIDKDKVDK

SEQ ID NO: 18 - protein, 12 aa, Homo sapiens
  MOD_RES (5)..(5): Ac-Lys
GFGGKFGVQTDR

SEQ ID NO: 19 - protein, 13 aa, Homo sapiens
  MOD_RES (6)..(6): Ac-Lys
TGFGGKFGVQSER

SEQ ID NO: 20 - protein, 13 aa, Homo sapiens
  MOD_RES (11)..(11): Ac-Lys
QDSAAVGFDYKEK

SEQ ID NO: 21 - protein, 10 aa, Homo sapiens
  MOD_RES (5)..(5): Ac-Lys
GFGGKYGVQK

SEQ ID NO: 22 - protein, 11 aa, Homo sapiens
  MOD_RES (10)..(10): Ac-Lys
EGVTPWASFKK

SEQ ID NO: 23 - protein, 10 aa, Homo sapiens
  MOD_RES (2)..(2): Ac-Lys
KKSKSKLEEK

SEQ ID NO: 24 - protein, 10 aa, Homo sapiens
  MOD_RES (6)..(6): Ac-Lys
KKSKSKLEEK

SEQ ID NO: 25 - protein, 12 aa, Homo sapiens
  MOD_RES (1)..(1): Ac-Lys
KEKGKLKVKEKK

SEQ ID NO: 26 - protein, 12 aa, Homo sapiens
  MOD_RES (5)..(5): Ac-Lys
KEKGKLKVKEKK

SEQ ID NO: 27 - protein, 12 aa, Homo sapiens
  MOD_RES (7)..(7): Ac-Lys
KEKGKLKVKEKK

SEQ ID NO: 28 - protein, 12 aa, Homo sapiens
  MOD_RES (11)..(11): Ac-Lys
KEKGKLKVKEKK

SEQ ID NO: 29 - protein, 12 aa, Homo sapiens
  MOD_RES (7)..(7): Ac-Lys
ALPSMDKYAVFK

SEQ ID NO: 30 - protein, 16 aa, Homo sapiens
  MOD_RES (11)..(11): Ac-Lys
GGQNSTAASTKYDVFR

SEQ ID NO: 31 - protein, 11 aa, Homo sapiens
  MOD_RES (10)..(10): Ac-Lys
IYEDGDDDMKR

SEQ ID NO: 32 - protein, 11 aa, Homo sapiens
  MOD_RES (3)..(3): Ac-Lys
SGKYYYQLNSK

SEQ ID NO: 33 - protein, 12 aa, Homo sapiens
  MOD_RES (12)..(12): Ac-Lys
QDAFANFANFSK

SEQ ID NO: 34 - protein, 26 aa, Homo sapiens
  MOD_RES (4)..(4): Ac-Lys
KREKRRKKKEQQQSEANELRNLAFKK

SEQ ID NO: 35 - protein, 20 aa, Homo sapiens
  MOD_RES (17)..(17): Ac-Lys
FSPNSSNPIIVSCGWDKLVK

SEQ ID NO: 36 - protein, 12 aa, Homo sapiens
  MOD_RES (2)..(2): Ac-Lys
EKLRRAAEEAER

SEQ ID NO: 37 - protein, 10 aa, Homo sapiens
  MOD_RES (1)..(1): Ac-Lys
KAAVAPGPWK

SEQ ID NO: 38 - protein, 16 aa, Homo sapiens
  MOD_RES (4)..(4): Ac-Lys
FAGKAGGKLAKAPGLK

SEQ ID NO: 39 - protein, 16 aa, Homo sapiens
  MOD_RES (8)..(8): Ac-Lys
FAGKAGGKLAKAPGLK

SEQ ID NO: 40 - protein, 16 aa, Homo sapiens
  MOD_RES (11)..(11): Ac-Lys
FAGKAGGKLAKAPGLK

SEQ ID NO: 41 - protein, 16 aa, Homo sapiens
  MOD_RES (16)..(16): Ac-Lys
SSSLGNLKKETSDGEK

SEQ ID NO: 42 - protein, 26 aa, Homo sapiens
  MOD_RES (21)..(21): Ac-Lys
LGGSPTSLGTWGSWIGPDHDKFSAMK

SEQ ID NO: 43 - protein, 17 aa, Homo sapiens
  MOD_RES (16)..(16): Ac-Lys
DAILDALENLTAEELKK

SEQ ID NO: 44 - protein, 16 aa, Homo sapiens
  MOD_RES (8)..(8): Ac-Lys
GFSGAGEKLFSSQYGK

SEQ ID NO: 45 - protein, 12 aa, Homo sapiens
  MOD_RES (12)..(12): Ac-Lys
MDVFMKGLSMAK

SEQ ID NO: 46 - protein, 23 aa, Homo sapiens
  MOD_RES (10)..(10): Ac-Lys
MGQKVTGGIKTVDMRDPTYRPLK

SEQ ID NO: 47 - protein, 23 aa, Homo sapiens
  MOD_RES (4)..(4): Ac-Lys
MGQKVTGGIKTVDMRDPTYRPLK

SEQ ID NO: 48 - protein, 17 aa, Homo sapiens
  MOD_RES (11)..(11): Ac-Lys
EWAIKQGILLKVAETIK

SEQ ID NO: 49 - protein, 15 aa, Homo sapiens
  MOD_RES (12)..(12): Ac-Lys
LPTPTSSVPAQKTER

SEQ ID NO: 50 - protein, 11 aa, Homo sapiens
  MOD_RES (8)..(8): Ac-Lys
VISSIEQKTER

SEQ ID NO: 51 - protein, 12 aa, Homo sapiens
  MOD_RES (12)..(12): Ac-Lys
YLAEFATGNDRK

SEQ ID NO: 52 - protein, 12 aa, Homo sapiens
  MOD_RES (11)..(11): Ac-Lys
YLAEVAAGDDKK

SEQ ID NO: 53 - protein, 12 aa, Homo sapiens
  MOD_RES (12)..(12): Ac-Lys
YLAEVAAGDDKK

SEQ ID NO: 54 - protein, 14 aa, Homo sapiens
  MOD_RES (8)..(8): Ac-Lys
VVSSIEQKTEGAEK

SEQ ID NO: 55 - protein, 15 aa, Homo sapiens
  MOD_RES (13)..(13): Ac-Lys
AIEINPDSAQPYKWR

SEQ ID NO: 56 - protein, 8 aa, Homo sapiens
  MOD_RES (7)..(7): Ac-Lys
LSGLSFKR

SEQ ID NO: 57 - protein, 20 aa, Homo sapiens
  MOD_RES (19)..(19): Ac-Lys
PSPAISVSVSAPAFYAPQKK

SEQ ID NO: 58 - protein, 20 aa, Homo sapiens
  MOD_RES (20)..(20): Ac-Lys
PSPAISVSVSAPAFYAPQKK

SEQ ID NO: 59 - protein, 23 aa, Homo sapiens
  MOD_RES (7)..(7): Ac-Lys
FTPVASKFSPGAPGGSGSQPNQK

SEQ ID NO: 60 - protein, 18 aa, Homo sapiens
  MOD_RES (13)..(13): Ac-Lys
LTEEETVCLDLDKVEAYR

SEQ ID NO: 61 - protein, 14 aa, Homo sapiens
  MOD_RES (8)..(8): Ac-Lys
AAVHLEGKIEQAQR

SEQ ID NO: 62 - protein, 11 aa, Homo sapiens
  MOD_RES (9)..(9): Ac-Lys
EVENSEDPKFR

SEQ ID NO: 63 - protein, 10 aa, Homo sapiens
  MOD_RES (4)..(4): Ac-Lys
TWEKLLLAAR

SEQ ID NO: 64 - protein, 10 aa, Homo sapiens
  MOD_RES (4)..(4): Ac-Lys
FHYKTDQGIK

SEQ ID NO: 65 - protein, 22 aa, Homo sapiens
  MOD_RES (21)..(21): Ac-Lys
TAVPPGLSSLPLTSVGNTGMKK

SEQ ID NO: 66 - protein, 11 aa, Homo sapiens
  MOD_RES (10)..(10): Ac-Lys
FVNVVPTFGKK

SEQ ID NO: 67 - protein, 11 aa, Homo sapiens
  MOD_RES (11)..(11): Ac-Lys
FVNVVPTFGKK

SEQ ID NO: 68 - protein, 26 aa, Homo sapiens
  MOD_RES (25)..(25): Ac-Lys
GYEVIYLTEPVDEYCIQALPEFDGKR

SEQ ID NO: 69 - protein, 12 aa, Homo sapiens
  MOD_RES (3)..(3): Ac-Lys
SEKFAFQAEVNR

SEQ ID NO: 70 - protein, 24 aa, Homo sapiens
  MOD_RES (5)..(5): Ac-Lys
RLQEKELDLSELDDPDSAYLQEAR

SEQ ID NO: 71 - protein, 16 aa, Homo sapiens
  MOD_RES (7)..(7): Ac-Lys
DLIKDLKSELSGNFEK

SEQ ID NO: 72 - protein, 14 aa, Homo sapiens
  MOD_RES (2)..(2): Ac-Lys
KKFPLKIFKKPLKK

SEQ ID NO: 73 - protein, 14 aa, Homo sapiens
  MOD_RES (6)..(6): Ac-Lys
KKFPLKIFKKPLKK

SEQ ID NO: 74 - protein, 14 aa, Homo sapiens
  MOD_RES (9)..(9): Ac-Lys
KKFPLKIFKKPLKK

SEQ ID NO: 75 - protein, 14 aa, Homo sapiens
  MOD_RES (13)..(13): Ac-Lys
KKFPLKIFKKPLKK

SEQ ID NO: 76 - protein, 14 aa, Homo sapiens
  MOD_RES (9)..(9): Ac-Lys
SEIDMNDIKAFYQK

SEQ ID NO: 77 - protein, 17 aa, Homo sapiens
  MOD_RES (16)..(16): Ac-Lys
AAYLQETGKPLDETLKK

SEQ ID NO: 78 - protein, 24 aa, Homo sapiens
  MOD_RES (17)..(17): Ac-Lys
GDLENAFLNLVQCIQNKPLYFADR

SEQ ID NO: 79 - protein, 16 aa, Homo sapiens
  MOD_RES (7)..(7): Ac-Lys
DLMTDLKSEISGDLAR

SEQ ID NO: 80 - protein, 13 aa, Homo sapiens
  MOD_RES (7)..(7): Ac-Lys
EHQAALKIQSAFR

SEQ ID NO: 81 - protein, 12 aa, Homo sapiens
  MOD_RES (8)..(8): Ac-Lys
VNINVPMKTEQK

SEQ ID NO: 82 - protein, 17 aa, Homo sapiens
  MOD_RES (12)..(12): Ac-Lys
SSTTVSSFANSKPGSAK

SEQ ID NO: 83 - protein, 9 aa, Homo sapiens
  MOD_RES (8)..(8): Ac-Lys
LPTEMEGKK

SEQ ID NO: 84 - protein, 33 aa, Homo sapiens
  MOD_RES (1)..(1): Ac-Lys
KLTPSAGKAMLTPKPAGGDEKDIKAFMGTPVQK

SEQ ID NO: 85 - protein, 33 aa, Homo sapiens; identical to SEQ ID NO: 84
  MOD_RES (14)..(14): Ac-Lys

SEQ ID NO: 86 - protein, 33 aa, Homo sapiens; identical to SEQ ID NO: 84
  MOD_RES (21)..(21): Ac-Lys

SEQ ID NO: 87 - protein, 12 aa, Homo sapiens
  MOD_RES (7)..(7): Ac-Lys
ESVNLGKSEGFK

SEQ ID NO: 88 - protein, 10 aa, Homo sapiens
  MOD_RES (1)..(1): Ac-Lys
KAASKTRAPR

SEQ ID NO: 89 - protein, 14 aa, Homo sapiens
  MOD_RES (3)..(3): Ac-Lys
ILKCAGNEDIITLR

SEQ ID NO: 90 - protein, 14 aa, Homo sapiens
  MOD_RES (13)..(13): Ac-Lys
AAEDDEDDDVDTKK

SEQ ID NO: 91 - protein, 14 aa, Homo sapiens
  MOD_RES (14)..(14): Ac-Lys
AAEDDEDDDVDTKK

SEQ ID NO: 92 - protein, 14 aa, Homo sapiens
  MOD_RES (11)..(11): Ac-Lys
SVEAAAELSAKDLK

SEQ ID NO: 93 - protein, 12 aa, Homo sapiens
  MOD_RES (11)..(11): Ac-Lys
AAEEEDEADPKR

SEQ ID NO: 94 - protein, 13 aa, Homo sapiens
  MOD_RES (13)..(13): Ac-Lys
HLFGQPNSAYDFK

SEQ ID NO: 95 - protein, 11 aa, Homo sapiens
  MOD_RES (9)..(9): Ac-Lys
SNILSNEMKAK

SEQ ID NO: 96 - protein, 15 aa, Homo sapiens
  MOD_RES (6)..(6): Ac-Lys
RVIGAKKDQYFLDKK

SEQ ID NO: 97 - protein, 15 aa, Homo sapiens
  MOD_RES (7)..(7): Ac-Lys
RVIGAKKDQYFLDKK

SEQ ID NO: 98 - protein, 15 aa, Homo sapiens
  MOD_RES (6)..(6): Ac-Lys
LQMEPKMQSKTKIYR

SEQ ID NO: 99 - protein, 15 aa, Homo sapiens
  MOD_RES (10)..(10): Ac-Lys
LQMEPKMQSKTKIYR

SEQ ID NO: 100 - protein, 15 aa, Homo sapiens
  MOD_RES (8)..(8): Ac-Lys
KKGKGLGKKRDPCLR

SEQ ID NO: 101 - protein, 9 aa, Homo sapiens
  MOD_RES (8)..(8): Ac-Lys
KGKGLGKKR

SEQ ID NO: 102 - protein, 10 aa, Homo sapiens
  MOD_RES (1)..(1): Ac-Lys
KKGKGLGKKR

SEQ ID NO: 103 - protein, 15 aa, Homo sapiens
  MOD_RES (2)..(2): Ac-Lys
KKGKGLGKKRDPCLR

SEQ ID NO: 104 - protein, 9 aa, Homo sapiens
  MOD_RES (3)..(3): Ac-Lys
KGKGLGKKR

SEQ ID NO: 105 - protein, 16 aa, Homo sapiens
  MOD_RES (11)..(11): Ac-Lys
IQSTVTQPGGKFAFFK

SEQ ID NO: 106 - protein, 10 aa, Homo sapiens
  MOD_RES (5)..(5): Ac-Lys
GGRGKGKGKK

SEQ ID NO: 107 - protein, 10 aa, Homo sapiens
  MOD_RES (7)..(7): Ac-Lys
GGRGKGKGKK

SEQ ID NO: 108 - protein, 33 aa, Homo sapiens
  MOD_RES (1)..(1): Ac-Lys
KPWKLAIQILKIAMVTIQLVLFGLSNQMVVAFK

SEQ ID NO: 109 - protein, 33 aa, Homo sapiens; identical to SEQ ID NO: 108
  MOD_RES (11)..(11): Ac-Lys

SEQ ID NO: 110 - protein, 18 aa, Homo sapiens
  MOD_RES (6)..(6): Ac-Lys
FSAYIKNSNPALNDNLEK

SEQ ID NO: 111 - protein, 13 aa, Homo sapiens
  MOD_RES (8)..(8): Ac-Lys
MKKDDSTKARPQK

SEQ ID NO: 112 - protein, 15 aa, Homo sapiens
  MOD_RES (5)..(5): Ac-Lys
HITAKEIETFVSLLR

SEQ ID NO: 113 - protein, 18 aa, Homo sapiens
  MOD_RES (6)..(6): Ac-Lys
FGIAAKYQIDPDACFSAK

SEQ ID NO: 114 - protein, 12 aa, Homo sapiens
  MOD_RES (8)..(8): Ac-Lys
GYGFGLIKLDLK

SEQ ID NO: 115 - protein, 12 aa, Homo sapiens
  MOD_RES (8)..(8): Ac-Lys
GFGFGLVKLDVK

SEQ ID NO: 116 - protein, 16 aa, Homo sapiens
  MOD_RES (4)..(4): Ac-Lys
ITLKETFLTSPEELYR

SEQ ID NO: 117 - protein, 12 aa, Homo sapiens
  MOD_RES (12)..(12): Ac-Lys
HKLDVTSVEDYK

SEQ ID NO: 118 - protein, 14 aa, Homo sapiens
  MOD_RES (10)..(10): Ac-Lys
AVDDGVNTFKVLTR

SEQ ID NO: 119 - protein, 14 aa, Homo sapiens
  MOD_RES (9)..(9): Ac-Lys
ELEVAEGGKAELER

SEQ ID NO: 120 - protein, 11 aa, Homo sapiens
  MOD_RES (2)..(2): Ac-Lys
EKYIDQEELNK

SEQ ID NO: 121 - protein, 15 aa, Homo sapiens
  MOD_RES (15)..(15): Ac-Lys
NPDDITNEEYGEFYK

SEQ ID NO: 122 - protein, 17 aa, Homo sapiens
  MOD_RES (16)..(16): Ac-Lys
CLELFSELAEDKENYKK

SEQ ID NO: 123 - protein, 17 aa, Homo sapiens
  MOD_RES (17)..(17): Ac-Lys
CLELFSELAEDKENYKK

SEQ ID NO: 124 - protein, 8 aa, Homo sapiens
  MOD_RES (2)..(2): Ac-Lys
AKFENLCK

SEQ ID NO: 125 - protein, 11 aa, Homo sapiens
  MOD_RES (10)..(10): Ac-Lys
LVNHFVEEFKR

SEQ ID NO: 126 - protein, 15 aa, Homo sapiens
  MOD_RES (15)..(15): Ac-Lys
NQVALNPQNTVFDAK

SEQ ID NO: 127 - protein, 11 aa, Homo sapiens
  MOD_RES (11)..(11): Ac-Lys
FGDPVVQSDMK

SEQ ID NO: 128 - protein, 12 aa, Homo sapiens
  MOD_RES (12)..(12): Ac-Lys
TWNDPSVQQDIK

SEQ ID NO: 129 - protein, 23 aa, Homo sapiens
  MOD_RES (21)..(21): Ac-Lys
AVEEKIEWLESHQDADIEDFKAK

SEQ ID NO: 130 - protein, 21 aa, Homo sapiens
  MOD_RES (18)..(18): Ac-Lys
LYGSAGPPPTGEEDTAEKDEL

SEQ ID NO: 131 - protein, 15 aa, Homo sapiens
  MOD_RES (15)..(15): Ac-Lys
NQLTSNPENTVFDAK

SEQ ID NO: 132 - protein, 11 aa, Homo sapiens
  MOD_RES (10)..(10): Ac-Lys
MVNHFIAEFKR

SEQ ID NO: 133 - protein, 12 aa, Homo sapiens
  MOD_RES (12)..(12): Ac-Lys
RFDDAVVQSDMK

SEQ ID NO: 134 - protein, 11 aa, Homo sapiens
  MOD_RES (8)..(8): Ac-Lys
YDDPEVQKDIK

SEQ ID NO: 135 - protein, 15 aa, Homo sapiens
  MOD_RES (8)..(8): Ac-Lys
ETGVDLTKDNMALQR

SEQ ID NO: 136 - protein, 22 aa, Homo sapiens
  MOD_RES (15)..(15): Ac-Lys
MEEFKDQLPADECNKLKEEISK

SEQ ID NO: 137 - protein, 13 aa, Homo sapiens
  MOD_RES (9)..(9): Ac-Lys
DGVVEITGKHEER

SEQ ID NO: 138 - protein, 16 aa, Homo sapiens
  MOD_RES (13)..(13): Ac-Lys
TLNDELEIIEGMKFDR

SEQ ID NO: 139 - protein, 15 aa, Homo sapiens
  MOD_RES (12)..(12): Ac-Lys
GYISPYFINTSKGQK

SEQ ID NO: 140 - protein, 16 aa, Homo sapiens
  MOD_RES (3)..(3): Ac-Lys
GQKCEFQDAYVLLSEK

SEQ ID NO: 141 - protein, 14 aa, Homo sapiens
  MOD_RES (13)..(13): Ac-Lys
CEFQDAYVLLSEKK

SEQ ID NO: 142 - protein, 23 aa, Homo sapiens
  MOD_RES (19)..(19): Ac-Lys
IQEIIEQLDVTTSEYEKEKLNER

SEQ ID NO: 143 - protein, 16 aa, Homo sapiens
  MOD_RES (16)..(16): Ac-Lys
CIPALDSLTPANEDQK

SEQ ID NO: 144 - protein, 11 aa, Homo sapiens
  MOD_RES (3)..(3): Ac-Lys
TLKIPAMTIAK

SEQ ID NO: 145 - protein, 9 aa, Homo sapiens
  MOD_RES (5)..(5): Ac-Lys
SIDLKDKYK

SEQ ID NO: 146 - protein, 29 aa, Homo sapiens
  MOD_RES (29)..(29): Ac-Lys
MKSEPDKADPFSFEGPEIVDCDGCTIDWK

SEQ ID NO: 147 - protein, 30 aa, Homo sapiens
  MOD_RES (30)..(30): Ac-Lys
MKSEPDKADPFSFEGPEIVDCDGCTIDWKK

SEQ ID NO: 148 - protein, 22 aa, Homo sapiens
  MOD_RES (21)..(21): Ac-Lys
DLGLSESGEDVNAAILDESGKK

SEQ ID NO: 149 - protein, 12 aa, Homo sapiens
  MOD_RES (5)..(5): Ac-Lys
IEVEKPFAIAKE

SEQ ID NO: 150 - protein, 10 aa, Homo sapiens
  MOD_RES (7)..(7): Ac-Lys
IGNSYFKEEK

SEQ ID NO: 151 - protein, 11 aa, Homo sapiens
  MOD_RES (10)..(10): Ac-Lys
SLHDALCVVKR

SEQ ID NO: 152 - protein, 13 aa, Homo sapiens
  MOD_RES (13)..(13): Ac-Lys
LTFSCLGGSDNFK

SEQ ID NO: 153 - protein, 22 aa, Homo sapiens
  MOD_RES (12)..(12): Ac-Lys
VDCTANTNTCNKYGVSGYPTLK

SEQ ID NO: 154 - protein, 13 aa, Homo sapiens
  MOD_RES (4)..(4): Ac-Lys
SYSKLLCGLLAER

SEQ ID NO: 155 - protein, 16 aa, Homo sapiens
  MOD_RES (16)..(16): Ac-Lys
YPIEHGIITNWDDMEK

SEQ ID NO: 156 - protein, 22 aa, Homo sapiens
  MOD_RES (11)..(11): Ac-Lys
HQGVMVGMGQKDSYVGDEAQSK

SEQ ID NO: 157 - protein, 11 aa, Homo sapiens
  MOD_RES (11)..(11): Ac-Lys
DSYVGDEAQSK

SEQ ID NO: 158 - protein, 17 aa, Homo sapiens
  MOD_RES (17)..(17): Ac-Lys
MAPYQGPDAVPGALDYK

SEQ ID NO: 159 - protein, 17 aa, Homo sapiens
  MOD_RES (10)..(10): Ac-Lys
KQAELDEEEKEIDAKLK

SEQ ID NO: 160 - protein, 21 aa, Homo sapiens
  MOD_RES (1)..(1): Ac-Lys
KKLTGIKHELQANCYEEVKDR

SEQ ID NO: 161 - protein, 21 aa, Homo sapiens
  MOD_RES (2)..(2): Ac-Lys
KKLTGIKHELQANCYEEVKDR

SEQ ID NO: 162 - protein, 19 aa, Homo sapiens
  MOD_RES (5)..(5): Ac-Lys
LTGIKHELQANCYEEVKDR

SEQ ID NO: 163 - protein, 19 aa, Homo sapiens
  MOD_RES (17)..(17): Ac-Lys
LTGIKHELQANCYEEVKDR

SEQ ID NO: 164 - protein, 14 aa, Homo sapiens
  MOD_RES (4)..(4): Ac-Lys
IASKYDHQAEEDLR

SEQ ID NO: 165 - protein, 12 aa, Homo sapiens
  MOD_RES (1)..(1): Ac-Lys
KYQSEFEEIEGR

SEQ ID NO: 166 - protein, 18 aa, Homo sapiens
  MOD_RES (17)..(17): Ac-Lys
SVSSNVASVSPIPAGSKK

SEQ ID NO: 167 - protein, 15 aa, Homo sapiens
  MOD_RES (12)..(12): Ac-Lys
FGSVPFTKNPEKYIK

SEQ ID NO: 168 - protein, 15 aa, Homo sapiens
  MOD_RES (15)..(15): Ac-Lys
FGSVPFTKNPEKYIK

SEQ ID NO: 169 - protein, 14 aa, Homo sapiens
  MOD_RES (14)..(14): Ac-Lys
RSIQFVDWCPTGFK

SEQ ID NO: 170 - protein, 16 aa, Homo sapiens
  MOD_RES (10)..(10): Ac-Lys
FETLQAQAGKHGDDLR

SEQ ID NO: 171 - protein, 12 aa, Homo sapiens
  MOD_RES (5)..(5): Ac-Lys
TLNNKFASFIDK

SEQ ID NO: 172 - protein, 14 aa, Homo sapiens
  MOD_RES (7)..(7): Ac-Lys
VTQKSYKVSTSGPR

SEQ ID NO: 173 - protein, 13 aa, Homo sapiens
  MOD_RES (5)..(5): Ac-Lys
MPETKWSLLQQQK

SEQ ID NO: 174 - protein, 13 aa, Homo sapiens
  MOD_RES (5)..(5): Ac-Lys
MLETKWSLLQQQK

SEQ ID NO: 175 - protein, 22 aa, Homo sapiens
  MOD_RES (12)..(12): Ac-Lys
SRAEAESMYQIKYEELQSLAGK

SEQ ID NO: 176 - protein, 10 aa, Homo sapiens
  MOD_RES (5)..(5): Ac-Lys
AVVVKKIETR

SEQ ID NO: 177 - protein, 10 aa, Homo sapiens
  MOD_RES (6)..(6): Ac-Lys
AVVVKKIETR

SEQ ID NO: 178 - protein, 14 aa, Homo sapiens
  MOD_RES (4)..(4): Ac-Lys
VTQKSYKVSTSGPR

SEQ ID NO: 179 - protein, 10 aa, Homo sapiens
  MOD_RES (8)..(8): Ac-Lys
LVEIDNGKQR

SEQ ID NO: 180 - protein, 40 aa, Homo sapiens
  MOD_RES (13)..(13): Ac-Lys
TVLCGTCGQPADKASASGSGAQVGGPISSGSSASSVTVTR

SEQ ID NO: 181 - protein, 18 aa, Homo sapiens
  MOD_RES (13)..(13): Ac-Lys
LYKEELEQTYHAKLENAR

SEQ ID NO: 182 - protein, 20 aa, Homo sapiens
  MOD_RES (5)..(5): Ac-Lys
ETPPKEVKKEVKKEEKKEVK

SEQ ID NO: 183 - protein, 20 aa, Homo sapiens
  MOD_RES (12)..(12): Ac-Lys
ETPPKEVKKEVKKEEKKEVK

SEQ ID NO: 184 - protein, 20 aa, Homo sapiens
  MOD_RES (13)..(13): Ac-Lys
ETPPKEVKKEVKKEEKKEVK

SEQ ID NO: 185 - protein, 20 aa, Homo sapiens
  MOD_RES (17)..(17): Ac-Lys
ETPPKEVKKEVKKEEKKEVK

SEQ ID NO: 186 - protein, 15 aa, Homo sapiens
  MOD_RES (1)..(1): Ac-Lys
KAKDKQAVSGQAAKK

SEQ ID NO: 187 - protein, 15 aa, Homo sapiens
  MOD_RES (3)..(3): Ac-Lys
KAKDKQAVSGQAAKK

SEQ ID NO: 188 - protein, 12 aa, Homo sapiens
  MOD_RES (6)..(6): Ac-Lys
VQIVYKPVDLSK

SEQ ID NO: 189 - protein, 17 aa, Homo sapiens
  MOD_RES (16)..(16): Ac-Lys
IGSLDNITHVPGGGNKK

SEQ ID NO: 190 - protein, 12 aa, Homo sapiens
  MOD_RES (1)..(1): Ac-Lys
KTMLAKFAEDDR

SEQ ID NO: 191 - protein, 15 aa, Homo sapiens
  MOD_RES (11)..(11): Ac-Lys
MNKKAGVAASKVKYK

SEQ ID NO: 192 - protein, 15 aa, Homo sapiens
  MOD_RES (13)..(13): Ac-Lys
MNKKAGVAASKVKYK

SEQ ID NO: 193 - protein, 20 aa, Homo sapiens
  MOD_RES (18)..(18): Ac-Lys
TLLQGSGCLAGIYLEDTKEK

SEQ ID NO: 194 - protein, 12 aa, Homo sapiens
  MOD_RES (12)..(12): Ac-Lys
GRLPLLAVCDYK

SEQ ID NO: 195 - protein, 8 aa, Homo sapiens
  MOD_RES (5)..(5): Ac-Lys
WAAEKLEK

SEQ ID NO: 196 - protein, 10 aa, Homo sapiens
  MOD_RES (7)..(7): Ac-Lys
EAQMAAKLER

SEQ ID NO: 197 - protein, 17 aa, Homo sapiens
  MOD_RES (10)..(10): Ac-Lys
DLSLEEIQKKLEAAEER

SEQ ID NO: 198 - protein, 28 aa, Homo sapiens
  MOD_RES (11)..(11): Ac-Lys
QAQILASEAEKAEQINQAAGEASAVLAK

SEQ ID NO: 199 - protein, 26 aa, Homo sapiens
  MOD_RES (26)..(26): Ac-Lys
SGPFGQIFRPDNFVFGQSGAGNNWAK

SEQ ID NO: 200 - protein, 15 aa, Homo sapiens
  MOD_RES (15)..(15): Ac-Lys
ALTVPELTQQMFDAK

SEQ ID NO: 201 - protein, 13 aa, Homo sapiens
  MOD_RES (4)..(4): Ac-Lys
TNEKVELQELNDR

SEQ ID NO: 202 - protein, 14 aa, Homo sapiens
  MOD_RES (13)..(13): Ac-Lys
KVESLQEEIAFLKK

SEQ ID NO: 203 - protein, 12 aa, Homo sapiens
  MOD_RES (11)..(11): Ac-Lys
DSYVGNEAQSKR

SEQ ID NO: 204 - protein, 12 aa, Homo sapiens
  MOD_RES (3)..(3): Ac-Lys
HGKYMACCLLYR

SEQ ID NO: 205 - protein, 12 aa, Homo sapiens
  MOD_RES (4)..(4): Ac-Lys
LDHKFDLMYAKR

SEQ ID NO: 206 - protein, 15 aa, Homo sapiens
  MOD_RES (14)..(14): Ac-Lys
TTGFYSGFSEVAEKR

SEQ ID NO: 207 - protein, 12 aa, Homo sapiens
  MOD_RES (6)..(6): Ac-Lys
RVVNGKVEYFLK

SEQ ID NO: 208 - protein, 17 aa, Homo sapiens
  MOD_RES (16)..(16): Ac-Lys
GGGNQVSLLNVVMDLKK

SEQ ID NO: 209 - protein, 15 aa, Homo sapiens
  MOD_RES (15)..(15): Ac-Lys
GKPEAAKKGVVKAEK

SEQ ID NO: 210 - protein, 17 aa, Homo sapiens
  MOD_RES (14)..(14): Ac-Lys
SESSSKSSQPLASKQEK

SEQ ID NO: 211 - protein, 17 aa, Homo sapiens
  MOD_RES (6)..(6): Ac-Lys
SESSSKSSQPLASKQEK

SEQ ID NO: 212 - protein, 13 aa, Homo sapiens
  MOD_RES (11)..(11): Ac-Lys
LGEMWSEQSAKDK

SEQ ID NO: 213 - protein, 22 aa, Homo sapiens
  MOD_RES (1)..(1): Ac-Lys
KVEEEDEEQEEEEEEEEEEEDE

SEQ ID NO: 214 - protein, 13 aa, Homo sapiens
  MOD_RES (11)..(11): Ac-Lys
RKAEGDAKGGKAK

SEQ ID NO: 215 - protein, 26 aa, Homo sapiens
  MOD_RES (12)..(12): Ac-Lys
EGNNPAENGDAKTDQAQKAEGAGDAK

SEQ ID NO: 216 - protein, 31 aa, Homo sapiens
  MOD_RES (31)..(31): Ac-Lys
ADAGKEGNNPAENGDAKTDQAQKAEGAGDAK

SEQ ID NO: 217 - protein, 17 aa, Homo sapiens
  MOD_RES (17)..(17): Ac-Lys
DASTLQSQKAEGTGDAK

SEQ ID NO: 218 - protein, 17 aa, Homo sapiens
  MOD_RES (10)..(10): Ac-Lys
FGEVVDCTIKTDPVTGR

SEQ ID NO: 219 - protein, 12 aa, Homo sapiens
  MOD_RES (8)..(8): Ac-Lys
YHQIGSGKCEIK

SEQ ID NO: 220 - protein, 15 aa, Homo sapiens
  MOD_RES (7)..(7): Ac-Lys
LTPLILKPFGNSISR

SEQ ID NO: 221 - protein, 11 aa, Homo sapiens
  MOD_RES (7)..(7): Ac-Lys
YGVAWEKYCQR

SEQ ID NO: 222 - protein, 17 aa, Homo sapiens
  MOD_RES (8)..(8): Ac-Lys
YMGPAGAKNLQSVVLSK

SEQ ID NO: 223 - protein, 17 aa, Homo sapiens
  MOD_RES (10)..(10): Ac-Lys
CRKAGLALSKHYKVYKR

SEQ ID NO: 224 - protein, 17 aa, Homo sapiens
  MOD_RES (13)..(13): Ac-Lys
CRKAGLALSKHYKVYKR

SEQ ID NO: 225 - protein, 17 aa, Homo sapiens
  MOD_RES (16)..(16): Ac-Lys
CRKAGLALSKHYKVYKR

SEQ ID NO: 226 - protein, 30 aa, Homo sapiens
  MOD_RES (29)..(29): Ac-Lys
IVGNKWEAYLGLLQAEYTEGDALDALGLKR

SEQ ID NO: 227 - protein, 11 aa, Homo sapiens
  MOD_RES (4)..(4): Ac-Lys
AWGKFGGAFCR

SEQ ID NO: 228 - protein, 13 aa, Homo sapiens
  MOD_RES (8)..(8): Ac-Lys
DRDSGRLKPALGR

SEQ ID NO: 229 - protein, 29 aa, Homo sapiens
  MOD_RES (8)..(8): Ac-Lys
KMNARQSKCYEMEDVLKHHWLYTSKYLQK

SEQ ID NO: 230 - protein, 26 aa, Homo sapiens
  MOD_RES (6)..(6): Ac-Lys
AYGASKTFGKAAGPSLSHTSGGTQSK

SEQ ID NO: 231 - protein, 26 aa, Homo sapiens
  MOD_RES (10)..(10): Ac-Lys
AYGASKTFGKAAGPSLSHTSGGTQSK

SEQ ID NO: 232 - protein, 19 aa, Homo sapiens
  MOD_RES (13)..(13): Ac-Lys
VVPIASLTPYQSKWTICAR

SEQ ID NO: 233 - protein, 21 aa, Homo sapiens
  MOD_RES (20)..(20): Ac-Lys
AIEDEGGNPDEIEITSEGNKK

SEQ ID NO: 234 - protein, 21 aa, Homo sapiens
  MOD_RES (21)..(21): Ac-Lys
AIEDEGGNPDEIEITSEGNKK

SEQ ID NO: 235 - protein, 31 aa, Homo sapiens
  MOD_RES (3)..(3): Ac-Lys
SQKEEDEQEDLTKDMEDPTPVPNIEEVVLPK

SEQ ID NO: 236 - protein, 8 aa, Homo sapiens
  MOD_RES (6)..(6): Ac-Lys
SFVVSKFR

SEQ ID NO: 237 - protein, 14 aa, Homo sapiens
  MOD_RES (6)..(6): Ac-Lys
LTDLDKAQLLEIAK

SEQ ID NO: 238 - protein, 15 aa, Homo sapiens
  MOD_RES (15)..(15): Ac-Lys
RLTDLDKAQLLEIAK

SEQ ID NO: 239 - protein, 7 aa, Homo sapiens
  MOD_RES (3)..(3): Ac-Lys
LYKEAEK

SEQ ID NO: 240 - protein, 10 aa, Homo sapiens
  MOD_RES (6)..(6): Ac-Lys
AEVGEKTEER

SEQ ID NO: 241 - protein, 20 aa, Homo sapiens
  MOD_RES (13)..(13): Ac-Lys
VWPSLQDRFSSLKGVPTEVK

SEQ ID NO: 242 - protein, 20 aa, Homo sapiens
  MOD_RES (20)..(20): Ac-Lys
VWPSLQDRFSSLKGVPTEVK

SEQ ID NO: 243 - protein, 14 aa, Homo sapiens
  MOD_RES (5)..(5): Ac-Lys
QSLAKVENACYALK

SEQ ID NO: 244 - protein, 12 aa, Homo sapiens
  MOD_RES (3)..(3): Ac-Lys
SGKFMNPTDQAR

SEQ ID NO: 245 - protein, 9 aa, Homo sapiens
  MOD_RES (7)..(7): Ac-Lys
LSAFAFKQE

SEQ ID NO: 246 - protein, 29 aa, Homo sapiens
  MOD_RES (22)..(22): Ac-Lys
NLEALALDLMEPEQAVDLTLPKVEAMNKR

SEQ ID NO: 247 - protein, 16 aa, Homo sapiens
  MOD_RES (2)..(2): Ac-Lys
RKKAEAAAAAMATPAR

SEQ ID NO: 248 - protein, 16 aa, Homo sapiens
  MOD_RES (3)..(3): Ac-Lys
RKKAEAAAAAMATPAR

SEQ ID NO: 249 - protein, 12 aa, Homo sapiens
  MOD_RES (11)..(11): Ac-Lys
FCNTTCLGAYKK

SEQ ID NO: 250 - protein, 17 aa, Homo sapiens
  MOD_RES (4)..(4): Ac-Lys
ECGKTFSGKSNLTEHEK

SEQ ID NO: 251 - protein, 17 aa, Homo sapiens
  MOD_RES (9)..(9): Ac-Lys
ECGKTFSGKSNLTEHEK

SEQ ID NO: 252 - protein, 12 aa, Homo sapiens
  MOD_RES (6)..(6): Ac-Lys
SSSQGKAYENKR

SEQ ID NO: 253 - protein, 15 aa, Homo sapiens
  MOD_RES (6)..(6): Ac-Lys
GATPGKALVATPGKK

SEQ ID NO: 254 - protein, 15 aa, Homo sapiens
  MOD_RES (14)..(14): Ac-Lys
GATPGKALVATPGKK

SEQ ID NO: 255 - protein, 15 aa, Homo sapiens
  MOD_RES (15)..(15): Ac-Lys
GATPGKALVATPGKK

SEQ ID NO: 256 - protein, 10 aa, Homo sapiens
  MOD_RES (7)..(7): Ac-Lys
GAAIPAKGAK

SEQ ID NO: 257 - protein, 12 aa, Homo sapiens
  MOD_RES (5)..(5): Ac-Lys
NLPYKVTQDELK

SEQ ID NO: 258 - protein, 9 aa, Homo sapiens
  MOD_RES (8)..(8): Ac-Lys
EALNSCNKR

SEQ ID NO: 259 - protein, 10 aa, Homo sapiens
  MOD_RES (9)..(9): Ac-Lys
VSELKEELKK

SEQ ID NO: 260 - protein, 13 aa, Homo sapiens
  MOD_RES (5)..(5): Ac-Lys
HLYTKDIDIHEVR

SEQ ID NO: 261 - protein, 14 aa, Homo sapiens
  MOD_RES (7)..(7): Ac-Lys
APQCLGKFIEIAAR

SEQ ID NO: 262 - protein, 29 aa, Homo sapiens
  MOD_RES (18)..(18): Ac-Lys
NQSQGYNQWQQGQFWGQKPWSQHYHQGYY

SEQ ID NO: 263 - protein, 21 aa, Homo sapiens
  MOD_RES (9)..(9): Ac-Lys
ADSLLAVVKREPAEQPGDGER

SEQ ID NO: 264 - protein, 19 aa, Homo sapiens
  MOD_RES (6)..(6): Ac-Lys
GLDWVKEEAPDILCLQETK

SEQ ID NO: 265 - protein, 11 aa, Homo sapiens
  MOD_RES (8)..(8): Ac-Lys
GQDGIGSKAEK

SEQ ID NO: 266 - protein, 13 aa, Homo sapiens
  MOD_RES (5)..(5): Ac-Lys
LYEEKTGNAWHSK

SEQ ID NO: 267 - protein, 13 aa, Homo sapiens
  MOD_RES (7)..(7): Ac-Lys
IAGMSQKTVTITK

SEQ ID NO: 268 - protein, 22 aa, Homo sapiens
  MOD_RES (7)..(7): Ac-Lys
NIHGYMKDIENYIQDGKDDYKK

SEQ ID NO: 269 - protein, 10 aa, Homo sapiens
  MOD_RES (4)..(4): Ac-Lys
KKKKGKKGKK

SEQ ID NO: 270 - protein, 10 aa, Homo sapiens
  MOD_RES (6)..(6): Ac-Lys
KKKKGKKGKK

SEQ ID NO: 271 - protein, 10 aa, Homo sapiens
  MOD_RES (7)..(7): Ac-Lys
KKKKGKKGKK

SEQ ID NO: 272 - protein, 10 aa, Homo sapiens
  MOD_RES (10)..(10): Ac-Lys
KKKKGKKGKK

SEQ ID NO: 273 - protein, 15 aa, Homo sapiens
  MOD_RES (12)..(12): Ac-Lys
KVQDGLSDIAEKFLK

SEQ ID NO: 274 - protein, 11 aa, Homo sapiens
  MOD_RES (4)..(4): Ac-Lys
GRSKGILNGQK

SEQ ID NO: 275 - protein, 11 aa, Homo sapiens
  MOD_RES (11)..(11): Ac-Lys
GRSKGILNGQK

SEQ ID NO: 276 - protein, 15 aa, Homo sapiens
  MOD_RES (8)..(8): Ac-Lys
TLAGSCGKFAPFEIK

SEQ ID NO: 277 - protein, 30 aa, Homo sapiens
  MOD_RES (29)..(29): Ac-Lys
MRSEPDDSDPFSFDGPEIMGCTGCQIDWKK

SEQ ID NO: 278 - protein, 30 aa, Homo sapiens; identical to SEQ ID NO: 277
  MOD_RES (30)..(30): Ac-Lys

SEQ ID NO: 279 - protein, 19 aa, Homo sapiens
  MOD_RES (3)..(3): Ac-Lys
SSKKAEPVKVLQKEKKRGK

SEQ ID NO: 280 - protein, 19 aa, Homo sapiens
  MOD_RES (4)..(4): Ac-Lys
SSKKAEPVKVLQKEKKRGK

SEQ ID NO: 281 - protein, 19 aa, Homo sapiens
  MOD_RES (15)..(15): Ac-Lys
SSKKAEPVKVLQKEKKRGK

SEQ ID NO: 282 - protein, 19 aa, Homo sapiens
  MOD_RES (19)..(19): Ac-Lys
SSKKAEPVKVLQKEKKRGK

SEQ ID NO: 283 - protein, 11 aa, Homo sapiens
  MOD_RES (11)..(11): Ac-Lys
QARELLAALQK

SEQ ID NO: 284 - protein, 14 aa, Homo sapiens
  MOD_RES (8)..(8): Ac-Lys
EGIKQLLKQGSVQK

SEQ ID NO: 285 - protein, 8 aa, Homo sapiens
  MOD_RES (4)..(4): Ac-Lys
GLTKAGAK

SEQ ID NO: 286 - protein, 9 aa, Homo sapiens
  MOD_RES (8)..(8): Ac-Lys
GLTKAGAKK

SEQ ID NO: 287 - protein, 8 aa, Homo sapiens
  MOD_RES (2)..(2): Ac-Lys
SKKKKPLK

SEQ ID NO: 288 - protein, 14 aa, Homo sapiens
  MOD_RES (14)..(14): Ac-Lys
HEQILVLDPPTDLK

SEQ ID NO: 289 - protein, 10 aa, Homo sapiens
  MOD_RES (2)..(2): Ac-Lys
SKFDNLYGCR

SEQ ID NO: 290 - protein, 10 aa, Homo sapiens
  MOD_RES (7)..(7): Ac-Lys
GGVVGIKVDK

SEQ ID NO: 291 - protein, 28 aa, Homo sapiens
  MOD_RES (27)..(27): Ac-Lys
YASICQQNGIVPIVEPEILPDGDHDLKR

SEQ ID NO: 292 - protein, 28 aa, Homo sapiens
  MOD_RES (15)..(15): Ac-Lys
ALSDHHIYLEGTLLKPNMVTPGHACTQK

SEQ ID NO: 293 - protein, 15 aa, Homo sapiens
  MOD_RES (14)..(14): Ac-Lys
GILAADESTGSIAKR

SEQ ID NO: 294 - protein, 10 aa, Homo sapiens
  MOD_RES (5)..(5): Ac-Lys
GYLDKLEPSK

SEQ ID NO: 295 - protein, 10 aa, Homo sapiens
  MOD_RES (8)..(8): Ac-Lys
ISEQSDAKLK

SEQ ID NO: 296 - protein, 11 aa, Homo sapiens
  MOD_RES (10)..(10): Ac-Lys
GSGGFGSTGKN

SEQ ID NO: 297 - protein, 14 aa, Homo sapiens
  MOD_RES (10)..(10): Ac-Lys
IGAEVYHNLKNVIK

SEQ ID NO: 298 - protein, 24 aa, Homo sapiens
  MOD_RES (2)..(2): Ac-Lys
EKYGKDATNVGDEGGFAPNILENK

SEQ ID NO: 299 - protein, 16 aa, Homo sapiens
  MOD_RES (3)..(3): Ac-Lys
SGKYDLDFKSPDDPSR

SEQ ID NO: 300 - protein, 12 aa, Homo sapiens
  MOD_RES (12)..(12): Ac-Lys
YISPDQLADLYK

SEQ ID NO: 301 - protein, 25 aa, Homo sapiens
  MOD_RES (25)..(25): Ac-Lys
SFIKDYPVVSIEDPFDQDDWGAWQK

SEQ ID NO: 302 - protein, 13 aa, Homo sapiens
  MOD_RES (5)..(5): Ac-Lys
AVNEKSCNCLLLK

SEQ ID NO: 303 - protein, 23 aa, Homo sapiens
  MOD_RES (8)..(8): Ac-Lys
SCNCLLLKVNQIGSVTESLQACK

SEQ ID NO: 304 - protein, 11 aa, Homo sapiens
  MOD_RES (8)..(8): Ac-Lys
TVDGPSGKLWR

SEQ ID NO: 305 - protein, 15 aa, Homo sapiens
  MOD_RES (8)..(8): Ac-Lys
VIPELNGKLTGMAFR

SEQ ID NO: 306 - protein, 12 aa, Homo sapiens
  MOD_RES (11)..(11): Ac-Lys
LEKPAKYDDIKK

SEQ ID NO: 307 - protein, 11 aa, Homo sapiens
  MOD_RES (6)..(6): Ac-Lys
FHGTVKAENGK

SEQ ID NO: 308 - protein, 18 aa, Homo sapiens
  MOD_RES (18)..(18): Ac-Lys
YFTLGLPTGSTPLGCYKK

SEQ ID NO: 309 - protein, 14 aa, Homo sapiens
  MOD_RES (8)..(8): Ac-Lys
FIIPNVVKYSPNCK

SEQ ID NO: 310 - protein, 17 aa, Homo sapiens
  MOD_RES (9)..(9): Ac-Lys
DQLIYNLLKEEQTPQNK

SEQ ID NO: 311 - protein, 17 aa, Homo sapiens
  MOD_RES (17)..(17): Ac-Lys
DQLIYNLLKEEQTPQNK

SEQ ID NO: 312 - protein, 13 aa, Homo sapiens
  MOD_RES (4)..(4): Ac-Lys
ATLKDQLIYNLLK

SEQ ID NO: 313 - protein, 15 aa, Homo sapiens
  MOD_RES (13)..(13): Ac-Lys
SADTLWDIQKDLKDL

SEQ ID NO: 314 - protein, 16 aa, Homo sapiens
  MOD_RES (10)..(10): Ac-Lys
HYGGLTGLNKAETAAK

SEQ ID NO: 315 - protein, 30 aa, Homo sapiens
  MOD_RES (19)..(19): Ac-Lys
SVVLMSHLGRPDGVPMPDKYSLEPVAVELK

SEQ ID NO: 316 - protein, 17 aa, Homo sapiens
  MOD_RES (11)..(11): Ac-Lys
GDLGIEIPAEKVFLAQK

SEQ ID NO: 317 - protein, 11 aa, Homo sapiens
  MOD_RES (11)..(11): Ac-Lys
CDENILWLDYK

SEQ ID NO: 318 - protein, 19 aa, Homo sapiens
  MOD_RES (18)..(18): Ac-Lys
GADFLVTEVENGGSLGSKK

SEQ ID NO: 319 - protein, 23 aa, Homo sapiens
  MOD_RES (23)..(23): Ac-Lys
GVNLPGAAVDLPAVSEKDIQDLK

SEQ ID NO: 320 - protein, 11 aa, Homo sapiens
  MOD_RES (10)..(10): Ac-Lys
GSGTAEVELKK

SEQ ID NO: 321 - protein, 27 aa, Homo sapiens
  MOD_RES (12)..(12): Ac-Lys
RQQEQQVPILEKFCFTPHTEEGCLSER

SEQ ID NO: 322 - protein, 22 aa, Homo sapiens
  MOD_RES (6)..(6): Ac-Lys
FLEIGKFDLSQNHPLGMAIFLK

SEQ ID NO: 323 - protein, 28 aa, Homo sapiens
  MOD_RES (19)..(19): Ac-Lys
DGLLENQTPEFFQDVCKPKYSGTLNLDR

SEQ ID NO: 324 - protein, 22 aa, Homo sapiens
  MOD_RES (11)..(11): Ac-Lys
LGMLSPEGTCKAFDTAGNGYCR

SEQ ID NO: 325 - protein, 22 aa, Homo sapiens
  MOD_RES (22)..(22): Ac-Lys
TGGAYGEDLGADYNLSQVCDGK

SEQ ID NO: 326 - protein, 36 aa, Homo sapiens
  MOD_RES (24)..(24): Ac-Lys
SLYQSAGVAPESFEYIEAHGTGTKVGDPQELNGITR

SEQ ID NO: 327 - protein, 14 aa, Homo sapiens
  MOD_RES (7)..(7): Ac-Lys
TPEAVQKLLEQGLR

SEQ ID NO: 328 - protein, 9 aa, Homo sapiens
  MOD_RES (6)..(6): Ac-Lys
EGVFAKEVR

SEQ ID NO: 329 - protein, 21 aa, Homo sapiens
  MOD_RES (11)..(11): Ac-Lys
FDASFFGVHPKQAHTMDPQLR

SEQ ID NO: 330 - protein, 14 aa, Homo sapiens
  MOD_RES (13)..(13): Ac-Lys
GLKPSCTIIPLMKK

SEQ ID NO: 331 - protein, 11 aa, Homo sapiens
  MOD_RES (11)..(11): Ac-Lys
VNYGAYINDFK

SEQ ID NO: 332 - protein, 11 aa, Homo sapiens
  MOD_RES (6)..(6): Ac-Lys
AVLIDKDQSPK

SEQ ID NO: 333 - protein, 15 aa, Homo sapiens
  MOD_RES (15)..(15): Ac-Lys
AAQAPSSFQLLYDLK

SEQ ID NO: 334 - protein, 19 aa, Homo sapiens
  MOD_RES (4)..(4): Ac-Lys
EREKLHAVNAEECNVLQGR

SEQ ID NO: 335 - protein, 14 aa, Homo sapiens
  MOD_RES (4)..(4): Ac-Lys
ASQKDFENSMNQVK

SEQ ID NO: 336 - protein, 15 aa, Homo sapiens
  MOD_RES (6)..(6): Ac-Lys
GDPGIKGDKGPPGGK

SEQ ID NO: 337 - protein, 15 aa, Homo sapiens
  MOD_RES (9)..(9): Ac-Lys
GDPGIKGDKGPPGGK

SEQ ID NO: 338 - protein, 15 aa, Homo sapiens
  MOD_RES (15)..(15): Ac-Lys
GDPGIKGDKGPPGGK

SEQ ID NO: 339 - protein, 9 aa, Homo sapiens
  MOD_RES (1)..(1): Ac-Lys
KGKGKGRKK

SEQ ID NO: 340 - protein, 9 aa, Homo sapiens
  MOD_RES (3)..(3): Ac-Lys
KGKGKGRKK

SEQ ID NO: 341 - protein, 9 aa, Homo sapiens
  MOD_RES (5)..(5): Ac-Lys
KGKGKGRKK

SEQ ID NO: 342 - protein, 9 aa, Homo sapiens
  MOD_RES (9)..(9): Ac-Lys
KGKGKGRKK

SEQ ID NO: 343 - protein, 19 aa, Homo sapiens
  MOD_RES (8)..(8): Ac-Lys
MGKEAVPKKGKEIPLASLR

SEQ ID NO: 344 - protein, 11 aa, Homo sapiens
  MOD_RES (5)..(5): Ac-Lys
LVSEKASIFEK

SEQ ID NO: 345 - protein, 14 aa, Homo sapiens
  MOD_RES (3)..(3): Ac-Lys
IDKTDYMVGSYGPR

SEQ ID NO: 346 - protein, 25 aa, Homo sapiens
  MOD_RES (2)..(2): Ac-Lys
MKFDIFEDYASPTTAAQTLLYTAAK

SEQ ID NO: 347 - protein, 12 aa, Homo sapiens
  MOD_RES (4)..(4): Ac-Lys
LDGKGEPKGAKR

SEQ ID NO: 348 - protein, 12 aa, Homo sapiens
  MOD_RES (8)..(8): Ac-Lys
LDGKGEPKGAKR

SEQ ID NO: 349 - protein, 12 aa, Homo sapiens
  MOD_RES (11)..(11): Ac-Lys
LDGKGEPKGAKR

SEQ ID NO: 350 - protein, 11 aa, Homo sapiens
  MOD_RES (9)..(9): Ac-Lys
FLNAENAQKFK

SEQ ID NO: 351 - protein, 22 aa, Homo sapiens
  MOD_RES (4)..(4): Ac-Lys
VAEKLEALSVKEETKEDAEEKQ

SEQ ID NO: 352 - protein, 22 aa, Homo sapiens
  MOD_RES (11)..(11): Ac-Lys
VAEKLEALSVKEETKEDAEEKQ

SEQ ID NO: 353 - protein, 19 aa, Homo sapiens
  MOD_RES (17)..(17): Ac-Lys
VCENIPIVLCGNKVDIKDR

SEQ ID NO: 354 - protein, 16 aa, Homo sapiens
  MOD_RES (11)..(11): Ac-Lys
FNVWDTAGQEKFGGLR

SEQ ID NO: 355 - protein, 13 aa, Homo sapiens
  MOD_RES (11)..(11): Ac-Lys
TQVAGGQLSFKGK

SEQ ID NO: 356 - protein, 15 aa, Homo sapiens
  MOD_RES (9)..(9): Ac-Lys
LLVHMGLLKSEDKVK

SEQ ID NO: 357 - protein, 12 aa, Homo sapiens
  MOD_RES (7)..(7): Ac-Lys
EQIGAAKEAKKK

SEQ ID NO: 358 - protein, 11 aa, Homo sapiens
  MOD_RES (11)..(11): Ac-Lys
QGNFFASPMLK

SEQ ID NO: 359 - protein, 16 aa, Homo sapiens
  MOD_RES (5)..(5): Ac-Lys
EIRTKKMSENKDKNGK

SEQ ID NO: 360 - protein, 16 aa, Homo sapiens
  MOD_RES (16)..(16): Ac-Lys
EIRTKKMSENKDKNGK

SEQ ID NO: 361 - protein, 11 aa, Homo sapiens
  MOD_RES (10)..(10): Ac-Lys
DLRIGKVAIKK

SEQ ID NO: 362 - protein, 17 aa, Homo sapiens
  MOD_RES (16)..(16): Ac-Lys
WAASLENLLEDPEGVKR

SEQ ID NO: 363 - protein, 25 aa, Homo sapiens
  MOD_RES (21)..(21): Ac-Lys
GNLYSFGCPEYGQLGHNSDGKFIAR

SEQ ID NO: 364 - protein, 15 aa, Homo sapiens
  MOD_RES (15)..(15): Ac-Lys
DQCREILKYVNEAVK

SEQ ID NO: 365 - protein, 14 aa, Homo sapiens
  MOD_RES (11)..(11): Ac-Lys
KMVNDAEPDTKKAK

SEQ ID NO: 366 - protein, 21 aa, Homo sapiens
  MOD_RES (19)..(19): Ac-Lys
YHYELLNYIDLPVLAIHGKQK

SEQ ID NO: 367 - protein, 13 aa, Homo sapiens
  MOD_RES (12)..(12): Ac-Lys
SDAGLESDTAMKK

SEQ ID NO: 368 - protein, 10 aa, Homo sapiens
  MOD_RES (6)..(6): Ac-Lys
QSSCGKFQTK

SEQ ID NO: 369 - protein, 22 aa, Homo sapiens
  MOD_RES (13)..(13): Ac-Lys
GNNNVMSNYEAYKPSTGAMGDR

SEQ ID NO: 370 - protein, 14 aa, Homo sapiens
  MOD_RES (13)..(13): Ac-Lys
HRLDLGEDYPSGKK

SEQ ID NO: 371 - protein, 12 aa, Homo sapiens
  MOD_RES (3)..(3): Ac-Lys
VAKIRLNLGKKR

SEQ ID NO: 372 - protein, 12 aa, Homo sapiens
  MOD_RES (11)..(11): Ac-Lys
VAKIRLNLGKKR

SEQ ID NO: 373 - protein, 13 aa, Homo sapiens
  MOD_RES (13)..(13): Ac-Lys
SSVNCPFSSQDMK

SEQ ID NO: 374 - protein, 10 aa, Homo sapiens
  MOD_RES (9)..(9): Ac-Lys
NFLYAWCGKR

SEQ ID NO: 375 - protein, 11 aa, Homo sapiens
  MOD_RES (11)..(11): Ac-Lys
ILADQQDKYMK

SEQ ID NO: 376 - protein, 11 aa, Homo sapiens
  MOD_RES (1)..(1): Ac-Lys
KGTEVQVDDIK

SEQ ID NO: 377 - protein, 11 aa, Homo sapiens
  MOD_RES (5)..(5): Ac-Lys
IAAYKSILQER

SEQ ID NO: 378 - protein, 23 aa, Homo sapiens
  MOD_RES (22)..(22): Ac-Lys
KKDQVTAQEIFQDNHEDGPTAKK

SEQ ID NO: 379 - protein, 8 aa, Homo sapiens
  MOD_RES (5)..(5): Ac-Lys
SGFGKFER

SEQ ID NO: 380 - protein, 12 aa, Homo sapiens
  MOD_RES (11)..(11): Ac-Lys
NQWQLSADDLKK

SEQ ID NO: 381 - protein, 12 aa, Homo sapiens
  MOD_RES (11)..(11): Ac-Lys
ASPPGDLQNPKR

SEQ ID NO: 382 - protein, 9 aa, Homo sapiens
  MOD_RES (2)..(2): Ac-Lys
NKFATFLQR

SEQ ID NO: 383 - protein, 11 aa, Homo sapiens
  MOD_RES (7)..(7): Ac-Lys
LETRGVKDVLK

SEQ ID NO: 384 - protein, 11 aa, Homo sapiens
  MOD_RES (10)..(10): Ac-Lys
CVDEGLLDCKR

SEQ ID NO: 385 - protein, 9 aa, Homo sapiens
  MOD_RES (9)..(9): Ac-Lys
GGNSLSDYK

SEQ ID NO: 386 - protein, 15 aa, Homo sapiens
  MOD_RES (15)..(15): Ac-Lys
ALLAYAFALAGNQDK

SEQ ID NO: 387 - protein, 22 aa, Homo sapiens
  MOD_RES (12)..(12): Ac-Lys
ATKESGLISTKKRKMVEMLEKK

SEQ ID NO: 388 - protein, 22 aa, Homo sapiens
  MOD_RES (21)..(21): Ac-Lys
ATKESGLISTKKRKMVEMLEKK

SEQ ID NO: 389 - protein, 22 aa, Homo sapiens
  MOD_RES (13)..(13): Ac-Lys
YVWLVYEQDRPLKCDEPILSNR

SEQ ID NO: 390 - protein, 20 aa, Homo sapiens
  MOD_RES (18)..(18): Ac-Lys
LYTLVLTDPDAPSRKDPKYR

SEQ ID NO: 391 - protein, 14 aa, Homo sapiens
  MOD_RES (4)..(4): Ac-Lys
EGVKTENNDHINLK

SEQ ID NO: 392 - protein, 33 aa, Homo sapiens
  MOD_RES (25)..(25): Ac-Lys
VYYFNHITNASQWERPSGNSSSGGKNGQGEPAR

SEQ ID NO: 393 - protein, 34 aa, Homo sapiens
  MOD_RES (27)..(27): Ac-Lys
HTGPGILSMANAGPNTNGSQFFICTAKTEWLDGK

SEQ ID NO: 394 - protein, 15 aa, Homo sapiens
  MOD_RES (13)..(13): Ac-Lys
TEWLDGKHVVFGKVK

SEQ ID NO: 395 - protein, 11 aa, Homo sapiens
  MOD_RES (5)..(5): Ac-Lys
GFGYKGSCFHR

SEQ ID NO: 396 - protein, 22 aa, Homo sapiens
  MOD_RES (7)..(7): Ac-Lys
HNGTGGKSIYGEKFEDENFILK

SEQ ID NO: 397 - protein, 15 aa, Homo sapiens
  MOD_RES (6)..(6): Ac-Lys
SIYGEKFEDENFILK

SEQ ID NO: 398 - protein, 11 aa, Homo sapiens
  MOD_RES (9)..(9): Ac-Lys
KLEEEEDGKLK

SEQ ID NO: 399 - protein, 16 aa, Homo sapiens
  MOD_RES (1)..(1): Ac-Lys
KQTTLAFKPIKKGKKR

SEQ ID NO: 400 - protein, 16 aa, Homo sapiens
  MOD_RES (12)..(12): Ac-Lys
KQTTLAFKPIKKGKKR

SEQ ID NO: 401 - protein, 16 aa, Homo sapiens
  MOD_RES (14)..(14): Ac-Lys
KQTTLAFKPIKKGKKR

SEQ ID NO: 402 - protein, 30 aa, Homo sapiens
  MOD_RES (29)..(29): Ac-Lys
ELASQPDVDGFLVGGASLKPEFVDIINAKQ

SEQ ID NO: 403 - protein, 10 aa, Homo sapiens
  MOD_RES (10)..(10): Ac-Lys
IAVAAQNCYK

SEQ ID NO: 404 - protein, 11 aa, Homo sapiens
  MOD_RES (4)..(4): Ac-Lys
DVAKPVIELYK

SEQ ID NO: 405 - protein, 13 aa, Homo sapiens
  MOD_RES (11)..(11): Ac-Lys
DVAKPVIELYKSR

SEQ ID NO: 406 - protein, 19 aa, Homo sapiens
  MOD_RES (6)..(6): Ac-Lys
IHAGPKFASYLTFSPSEVK

SEQ ID NO: 407 - protein, 15 aa, Homo sapiens
  MOD_RES (15)..(15): Ac-Lys
EISLWFKPEELVDYK

SEQ ID NO: 408 - protein, 15 aa, Homo sapiens
  MOD_RES (10)..(10): Ac-Lys
ECSPWMSDFKVEFLR

SEQ ID NO: 409 - protein, 12 aa, Homo sapiens
  MOD_RES (6)..(6): Ac-Lys
KEGRGKGKGKKK

SEQ ID NO: 410 - protein, 12 aa, Homo sapiens
  MOD_RES (11)..(11): Ac-Lys
KEGRGKGKGKKK

SEQ ID NO: 411 - protein, 12 aa, Homo sapiens
  MOD_RES (12)..(12): Ac-Lys
KEGRGKGKGKKK

SEQ ID NO: 412 - protein, 11 aa, Homo sapiens
  MOD_RES (4)..(4): Ac-Lys
LLLKYGDAWVK

SEQ ID NO: 413 - protein, 12 aa, Homo sapiens
  MOD_RES (4)..(4): Ac-Lys
LLMKFGDEPVTK

SEQ ID NO: 414 - protein, 15 aa, Homo sapiens
  MOD_RES (12)..(12): Ac-Lys
AYVDDTPAEQMKAER

SEQ ID NO: 415 - protein, 16 aa, Homo sapiens
  MOD_RES (15)..(15): Ac-Lys
SVVNMEWDKIWAFNKK

SEQ ID NO: 416 - protein, 16 aa, Homo sapiens
  MOD_RES (16)..(16): Ac-Lys
SVVNMEWDKIWAFNKK

SEQ ID NO: 417 - protein, 12 aa, Homo sapiens
  MOD_RES (3)..(3): Ac-Lys
SYKPVFWSPSSR

SEQ ID NO: 418 - protein, 9 aa, Homo sapiens
  MOD_RES (8)..(8): Ac-Lys
IATGSFLKR

SEQ ID NO: 419 - protein, 11 aa, Homo sapiens
  MOD_RES (5)..(5): Ac-Lys
NLTIKTESTLK

SEQ ID NO: 420 - protein, 20 aa, Homo sapiens
  MOD_RES (11)..(11): Ac-Lys
TTQFSGTLGEKFEENTADGR

SEQ ID NO: 421 - protein, 20 aa, Homo sapiens
  MOD_RES (11)..(11): Ac-Lys
TTQFSCTLGEKFEETTADGR

SEQ ID NO: 422 - protein, 14 aa, Homo sapiens
  MOD_RES (13)..(13): Ac-Lys
GSTLTSPCQDFGKR

SEQ ID NO: 423 - protein, 12 aa, Homo sapiens
  MOD_RES (11)..(11): Ac-Lys
KLEEEGEQFVKK

SEQ ID NO: 424 - protein, 12 aa, Homo sapiens
  MOD_RES (11)..(11): Ac-Lys
QQIGSSAMPYKR

SEQ ID NO: 425 - protein, 24 aa, Homo sapiens
  MOD_RES (22)..(22): Ac-Lys
LCNKPIQSLPNMDSIFSEALLKMR

SEQ ID NO: 426 - protein, 12 aa, Homo sapiens
  MOD_RES (8)..(8): Ac-Lys
MITSVLEKEASK

SEQ ID NO: 427 - protein, 11 aa, Homo sapiens
  MOD_RES (7)..(7): Ac-Lys
GWYQYDKPLGR

SEQ ID NO: 428 - protein, 28 aa, Homo sapiens
  MOD_RES (23)..(23): Ac-Lys
THTQDAVPLTLGQEFSGYVQQVKYAMTR

SEQ ID NO: 429 - protein, 25 aa, Homo sapiens
  MOD_RES (17)..(17): Ac-Lys
TGIEQGSDAGYLCESQKFGELVMTK

SEQ ID NO: 430 - protein, 12 aa, Homo sapiens
  MOD_RES (7)..(7): Ac-Lys
GQQQVFKGLNDK

SEQ ID NO: 431 - protein, 32 aa, Homo sapiens
  MOD_RES (3)..(3): Ac-Lys
VLKEVEAVIPDHCIFASNTSALPISEIAAVSK

SEQ ID NO: 432 - protein, 11 aa, Homo sapiens
  MOD_RES (10)..(10): Ac-Lys
GFYIYQEGVKR

(19) United States
(12) Patent Application Publication
(10) Pub. No.: US 2009/0124023 A1
(43) Pub. Date: May 14, 2009

(54) Reagens for the Detection of Protein Acetylation Signaling Pathways

(76) Inventors: Ailan Guo, Burlington, MA (US); Ting-Lei Gu, Woburn, MA (US); Jeffrey Mitchell, Nashua, NH (US); Peter Hornbeck, Magnolia, MA (US)

Correspondence Address: Simona Levi-Minzi, Ph.D.;General Counsel, CELL SIGNALING TECHNOLOGY, INC., 3 Trask Lane, Danvers, MA, 01923, US

(21) Appl. No.: 12/227,321

(22) PCT Filed: May 11, 2007
(86) PCT No.: PCT/US2007/011444

§ 371 Date: Nov. 12, 2008

Related U.S. Application Data
(60) Provisional application No. 60/799,962, filed on May 12, 2006.

(51) Int. Cl. G01N 33/566; C07K 16/18

(52) U.S. Cl. 436/501; 530/387.9

(57) ABSTRACT

The invention discloses 432 novel acetylation sites identified in signal transduction proteins and pathways underlying human protein acetylation signaling pathways, and provides acetylation-site specific antibodies and heavy-isotope labeled peptides (AQUA peptides) for the selective detection and quantification of these acetylated sites/proteins, as well as methods of using the reagents for such purpose. Among the acetylation sites identified are sites occurring in the following protein types: Acetyltransferases, Adaptor/Scaffold proteins, Actin binding proteins, Adhesion proteins, Apoptosis proteins, Calcium-binding proteins, Cell Cycle Regulation proteins, Cell Surface proteins, DNA binding proteins, DNA replication proteins, Channel proteins, Chaperone proteins, Cellular Metabolism enzymes, Cytoskeletal proteins, DNA repair proteins, Endoplasmic reticulum proteins, Enzyme proteins, G protein and GTPase Activating proteins, Guanine Nucleotide Exchange Factors, Helicase proteins, Isomerase proteins, Extracelluar matrix proteins, Hydrolases, Ligase proteins, Lipid kinases, Inhibtor proteins, Lipid Binding proteins and Lyases.

### RELATED APPLICATIONS

This application claims the benefit of, and priority to, U.S. Ser. No. 60/799,962, filed May 12, 2006, presently pending, the disclosure of which is incorporated herein, in its entirety, by reference.

### FIELD OF THE INVENTION

The invention relates generally to antibodies and peptide reagents for the detection of protein acetylation, and to protein acetylation in cancer.

### BACKGROUND OF THE INVENTION

The activation of proteins by post-translational modification is an important cellular mechanism for regulating most aspects of biological organization and control, including growth, development, homeostasis, and cellular communication. Protein phosphorylation, for example, plays a critical role in the etiology of many pathological conditions and diseases, including cancer, developmental disorders, autoimmune diseases, and diabetes. Yet, in spite of the importance of protein modification, it is not yet well understood at the molecular level, due to the extraordinary complexity of signaling pathways, and the slow development of technology necessary to unravel it.

Protein phosphorylation on a proteome-wide scale is extremely complex as a result of three factors: the large number of modifying proteins, e.g. kinases, encoded in the genome, the much larger number of sites on substrate proteins that are modified by these enzymes, and the dynamic nature of protein expression during growth, development, disease states, and aging. The human genome, for example, encodes over 520 different protein kinases, making them the most abundant class of enzymes known. See Hunter, Nature 411: 355-65 (2001). Most kinases phosphorylate many different substrate proteins, at distinct tyrosine, serine, and/or threonine residues. Indeed, it is estimated that one-third of all proteins encoded by the human genome are phosphorylated, and many are phosphorylated at multiple sites by different kinases. See Graves et al., Pharmacol. Ther. 82:111-21 (1999).

Many of these phosphorylation sites regulate critical biological processes and may prove to be important diagnostic or therapeutic targets for molecular medicine. For example, of the more than 100 dominant oncogenes identified to date, 46 are protein kinases. See Hunter, supra. Understanding which proteins are modified by these kinases will greatly expand our understanding of the molecular mechanisms underlying oncogenic transformation. Therefore, the identification of, and ability to detect, phosphorylation sites on a wide variety of cellular proteins is crucially important to understanding the key signaling proteins and pathways implicated in the progression of diseases like cancer.

Likewise, protein acetylation plays a complex and critical role in the regulation of biological processes and may prove to be important to diagnostic or therapeutic targets for molecular medicine. Protein acetylation on lysine residues is a dynamic, reversible and highly regulated chemical modification. Historically, histone was perceived as the most important substrate of acetylation, if not the sole substrate. It was proposed 40 years ago that structural modification of histones by acetylation plays an important role in chromatin remodeling and gene expression. Two groups of enzymes, histone deacetylases (HDACs) and histone acetyltransferases (HATs), are responsible for deacetylating and acetylating the histones.

Recent studies have revealed that HDACs are involved in a much broader assay of biological processes. For example, HDAC6 has been implicated in the regulation of microtubules, growth factor-induced chemotaxis and misfolded protein stress response. See Cohen et al., Science, vol 245:42 (2004). Consistant with these non-histone functions, HDAC6 is mainly located to the cytoplasm.

A growing list of acetylated proteins is currently available. It shows that both cytoplasmic and nuclear proteins can undergo reversible acetylation, and protein acetylation can have the following effects on its function: 1) Protein stability. Both acetylation and ubiquitylation often occur on the same lysine, competition between these two modifications affects the protein stability. It has been shown that HDACs can decrease the half-life of some proteins by exposing the lysine for ubiquitylation. 2) Protein-protein interactions. It has been shown that acetylation induces STAT3 dimerization and subsequently nuclear translocation. In the case of nuclear DNA-damage-response protein Ku70, the deacetylated form of Ku70 sequesters BAX, the pro-apoptotic protein, in the cytoplasm and protects cells from apoptosis. In response to apoptotic stimuli, Ku70 becomes acetylated and subsequently releases Bax from its sequestration, leading to translocation of BAX to the mitochondria and activation of apoptotic cascade. 3) Protein translocation. As described for STAT3 and BAX, reversible acetylation affects the subcellular localization. In the case of STAT3, its nuclear localization signal contains lysine residues that favor nuclear retension when acetylated. 4) DNA binding. It have been shown that acetylation of p53 regulates its stability, its DNA binding and its transcriptional activity. Similarly, the DNA binding affinity of NF-kB and its transcriptional activation are also regulated by HATs and HDACs. See Minucci et al., Nature Cancer Reviews, 6: 38-51 (2005).

HATs and HDACs have been linked to pathogenesis of cancer. Specific HATs (p300 and CBP) are targets of viral oncoproteins (adenoviral E1A, human papilloma virus E6 and SV40 T antigen). See Eckner, R. et al., Cold Spring Harb. Symp. Quant. Biol., 59: 85-95 (1994). Structural alterations in HATs, including translocation, amplifications, deletions and point mutations have been found in various human cancers. See Iyer, N G. et al., Oncogene, 23: 4225-4231 (2004). For HDACs, increased expression of HDAC1 has been detected in gastric cancers, oesophageal squamous cell carcinoma, and prostate cancer. See Halkidou, K. et al., Prostate 59: 177-189 (2004). Increased expression of HDAC2 has been detected in colon cancer and has been shown to interact functionally with Wnt pathway. Knockdown of HDAC2 by siRNA in colon cancer cells resulted in cell death. See Zhu, P. et al., Cancer Cell, 5: 455-463 (2004). Increased expression of HDAC6 has been linked to better survival in breast cancer, See Zhang, Z. et al., Clin. Cancer Res., 10: 6962-6968 (2004), while reduced expression of HDAC5 and 10 have been associated with poor prognosis in lung cancer patients. See Osada, H. et al., Cancer, 112: 26-32 (2004).

HDAC inhibitors (HDACi) are promising new targeted anti-cancer agents, and first-generation HDACi in several clinical trials show significant activity against a spectrum of both hematologic and solid tumors at doses that are well tolerated by the patients. See Drummond, D C. et al., Annu. Rev. Pharmacol. Toxicol., 45: 495-528 (2005). However, the relationship between the toxicity of HDACi and their pharmacokinetic properties is still largely unknown, which makes it difficult to optimize HDACi treatment. More importantly the key targets for HDACi action are unknown. This makes it difficult to select patients who are most likely to respond to HDACi. Proposed surrogate markers, like measuring the level of acetylated histone from blood cells before and after treatment, should be serve as indicators of effectiveness, but these need to be validated clinically yet and do not always correlated with pharmacokinetic profile. Therefore, to identify the entire spectrum of acetylated proteins deserves a much more systematic experimental strategy which would optimally a dynamic map of the acetylated proteins and their functions.

Despite the identification of a few key molecules involved in protein acetylation signaling pathways, the vast majority of signaling protein changes underlying these pathways remains unknown. There is, therefore, relatively scarce information about acetylation-driven signaling pathways and acetylation sites relevant to the pathogenesis of Cancer. This has hampered a complete and accurate understanding of how protein activation within signaling pathways may be driving different human diseases, including cancer.

Accordingly, there is a continuing and pressing need to unravel the molecular mechanisms of acetylation-driven oncogenesis in cancer by identifying the downstream signaling proteins mediating cellular transformation. Identifying particular acetylation sites on such signaling proteins and providing new reagents, such as acetyl-specific antibodies and AQUA peptides, to detect and quantify them remains particularly important to advancing our understanding of the biology of this pathway. Moreover, identification of downstream signaling molecules and acetylation sites involved in acetylation signaling and development of new reagents to detect and quantify these sites and proteins may lead to improved diagnostic/prognostic markers, as well as novel drug targets, for the detection and treatment of cancer.

### SUMMARY OF THE INVENTION

The invention discloses 432 novel acetylation sites identified in signal transduction proteins and pathways relevant to protein acetylation signaling and provides new reagents, including acetylation-site specific antibodies and AQUA peptides, for the selective detection and quantification of these acetylated sites/proteins. Also provided are methods of using the reagents of the invention for the detection and quantification of the disclosed acetylation sites.

### BRIEF DESCRIPTION OF THE DRAWINGS

FIG. 1—Is a diagram broadly depicting the immunoaffinity isolation and mass-spectrometric characterization methodology (IAP) employed to identify the novel acetylation sites disclosed herein.

FIG. 2—Is a table (corresponding to Table 1) enumerating the novel protein acetylation signaling sites disclosed herein: Column A=the name of the parent protein; Column B=the SwissProt accession number for the protein (human sequence); Column C=the protein type/classification; Column D=the lysine residue (in the parent protein amino acid sequence) at which acetylation occurs within the acetylation site; Column E=the acetylation site sequence encompassing the acetylatable residue (residue at which acetylation occurs (and corresponding to the respective entry in Column D) appears in lowercase; Column F=the type of disease in which the acetylation site was discovered; and Column G=the cell type(s) in which the acetylation site was discovered.

FIG. 3—is an exemplary mass spectrograph depicting the detection of the lysine 235 acetylation site in CTTN (see Row 19 in FIG. 2/Table 1), as further described in Example 1 (red and blue indicate ions detected in MS/MS spectrum); K* indicates the acetylated lysine (shown as uppercase “K” in FIG. 2).

FIG. 4—is an exemplary mass spectrograph depicting the detection of the lysine 689 acetylation site in CULL (see Row 82 in FIG. 2/Table 1), as further described in Example 1 (red and blue indicate ions detected in MS/MS spectrum); K* indicates the acetylated lysine (shown as uppercase “K” in FIG. 2).

FIG. 5—is an exemplary mass spectrograph depicting the detection of the lysine 11 acetylation site in SUM02 (see Row 392 in FIG. 2/Table 1), as further described in Example 1 (red and blue indicate ions detected in MS/MS spectrum); K* indicates the acetylated lysine (shown as uppercase “K” in FIG. 2).

FIG. 6—is an exemplary mass spectrograph depicting the detection of the lysine 82 acetylation site in PPIA (see Row 398 in FIG. 2/Table 1), as further described in Example 1 (red and blue indicate ions detected in MS/MS spectrum); K* indicates the acetylated lysine (shown as uppercase “K” in FIG. 2).

FIG. 7—is an exemplary mass spectrograph depicting the detection of the lysine 53 acetylation site in STMN1 (see Row 198 in FIG. 2/Table 1), as further described in Example 1 (red and blue indicate ions detected in MS/MS spectrum); K* indicates the acetylated lysine (shown as uppercase “K” in FIG. 2).

FIG. 8—is an exemplary mass spectrograph depicting the detection of the lysine 436 acetylation site in FASN (see Row 328 in FIG. 2/Table 1), as further described in Example 1 (red and blue indicate ions detected in MS/MS spectrum); K* indicates the acetylated lysine (shown as lowercase “K” in FIG. 2).

FIG. 9—is an exemplary mass spectrograph depicting the detection of the lysine 70 acetylation site in FASN (see Row 330 in FIG. 2/Table 1), as further described in Example 1 (red and blue indicate ions detected in MS/MS spectrum); K* indicates the acetylated lysine (shown as lowercase “K” in FIG. 2).

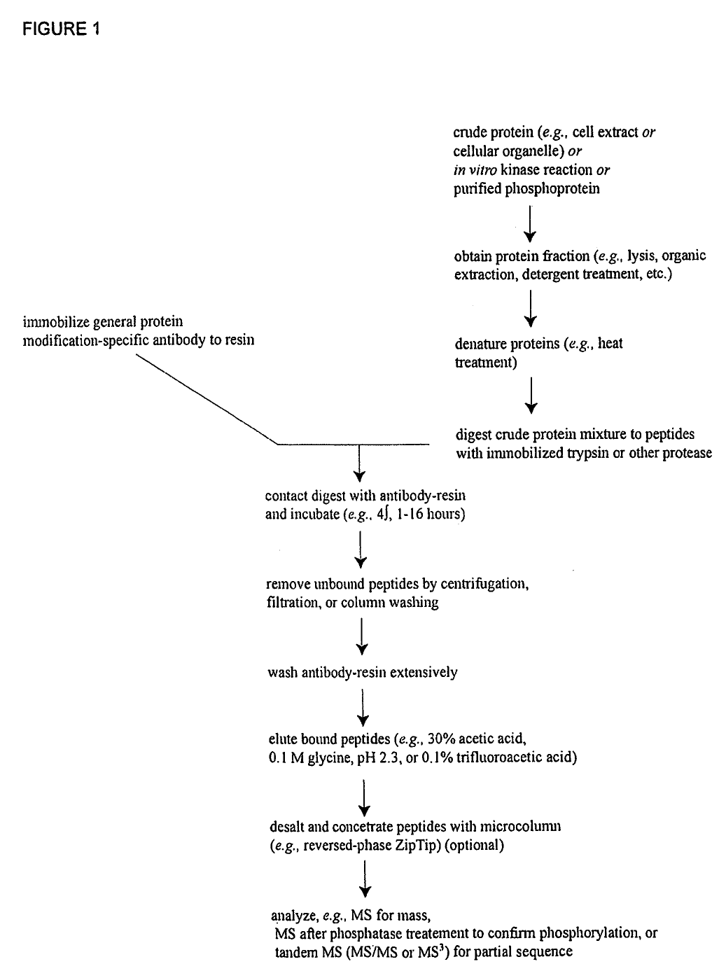

### DETAILED DESCRIPTION OF THE INVENTION

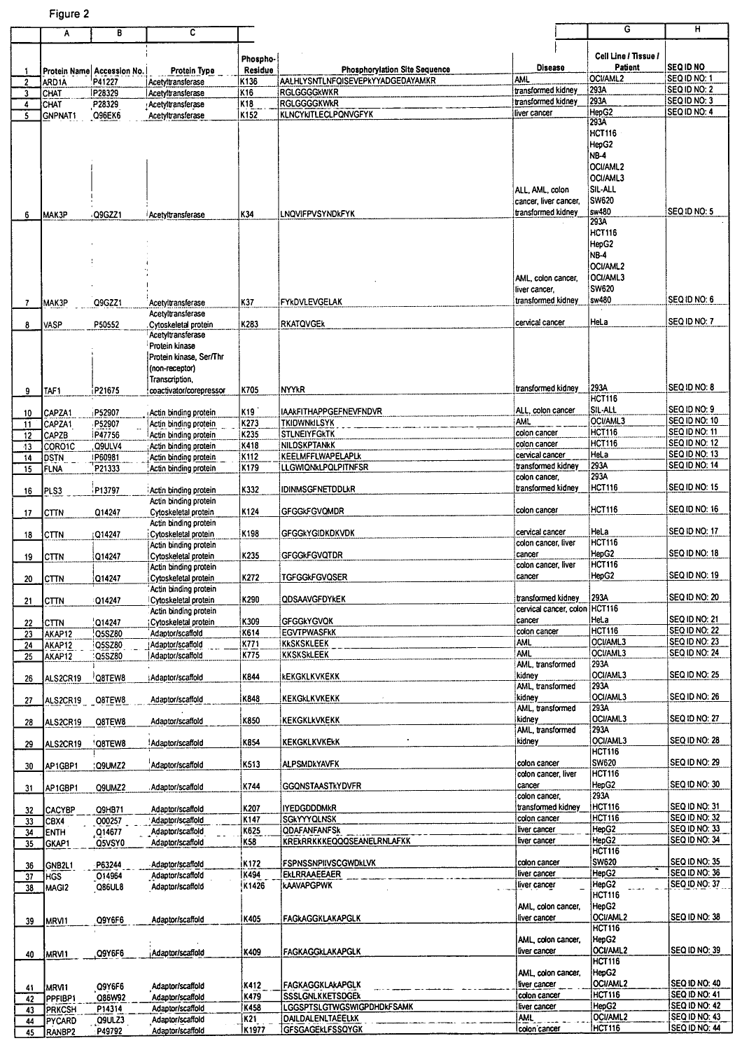

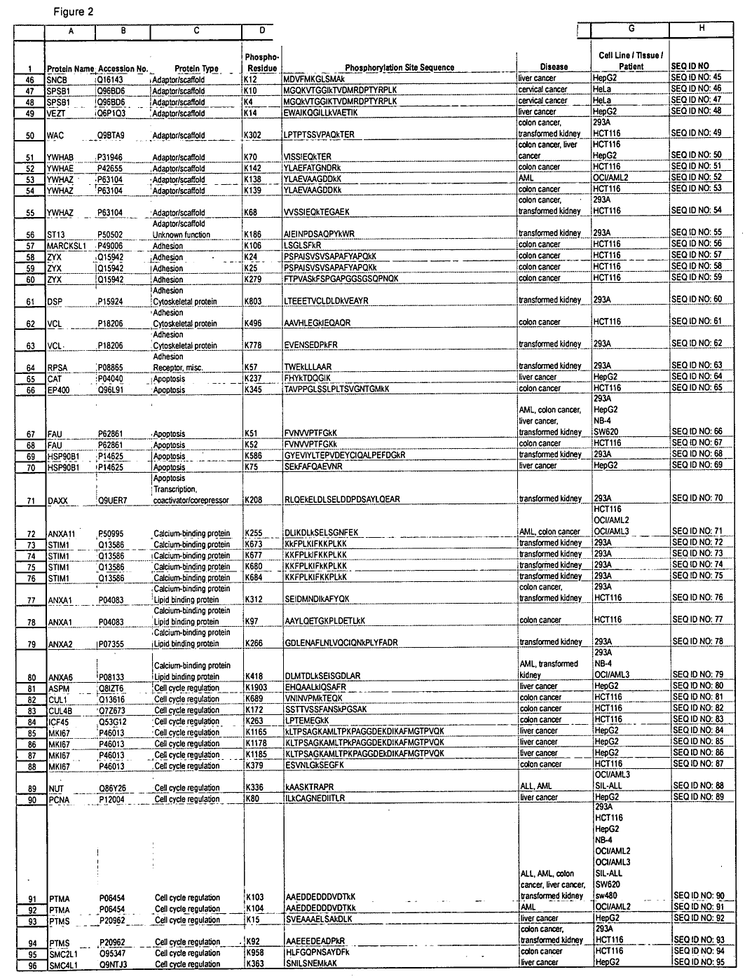

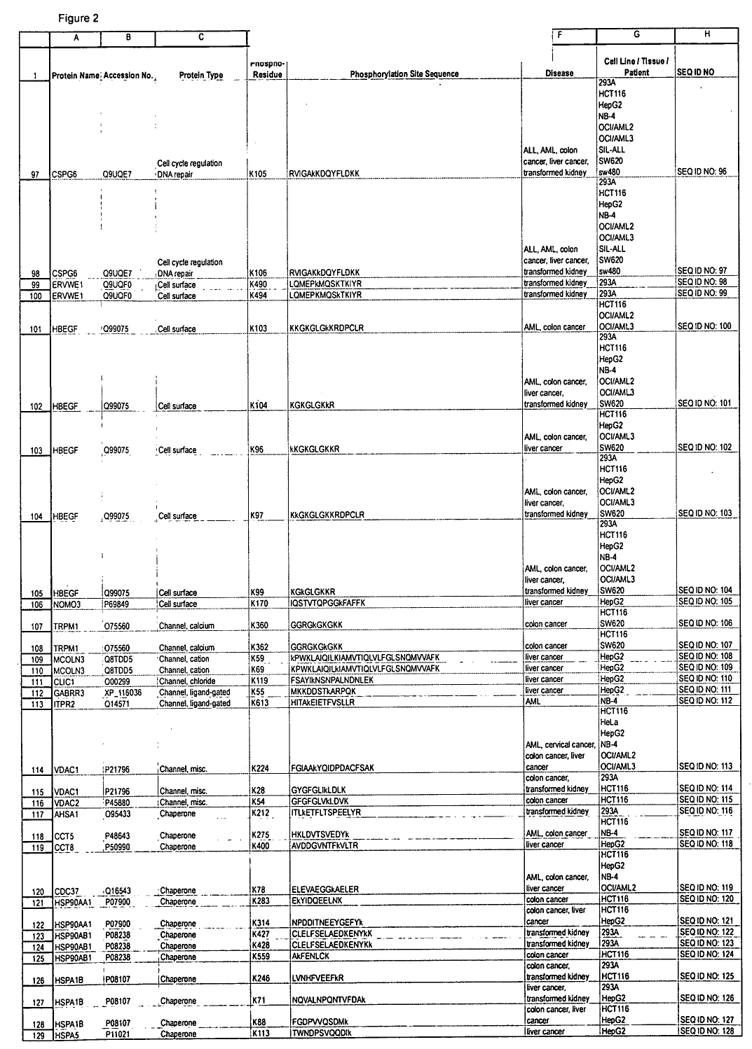

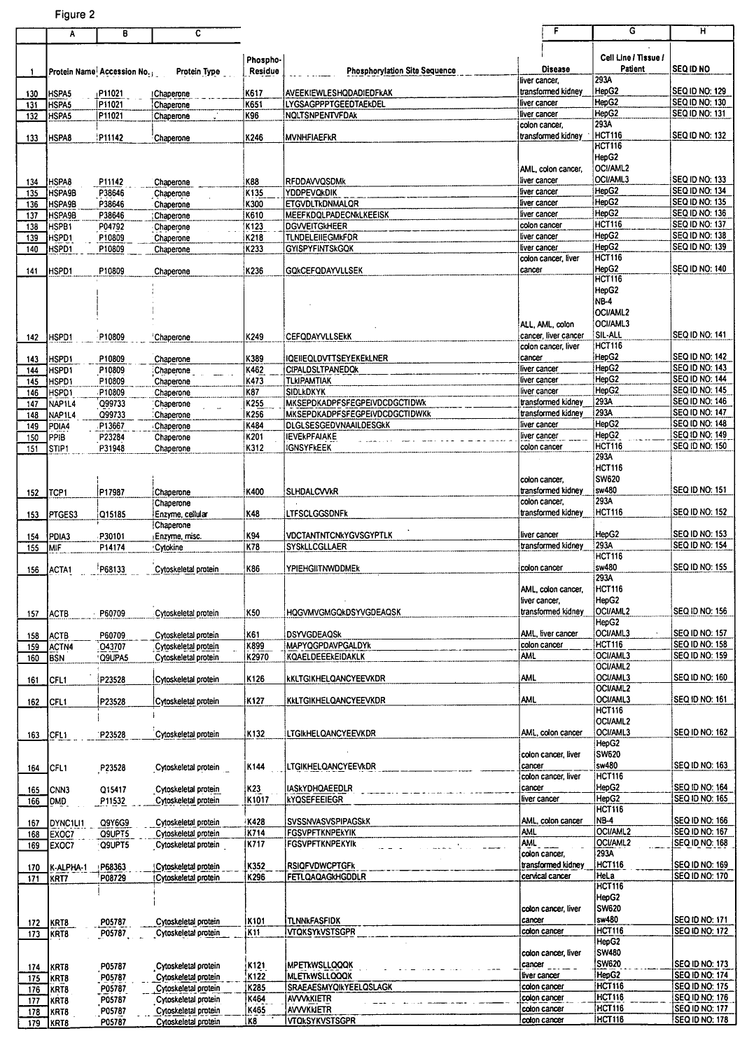

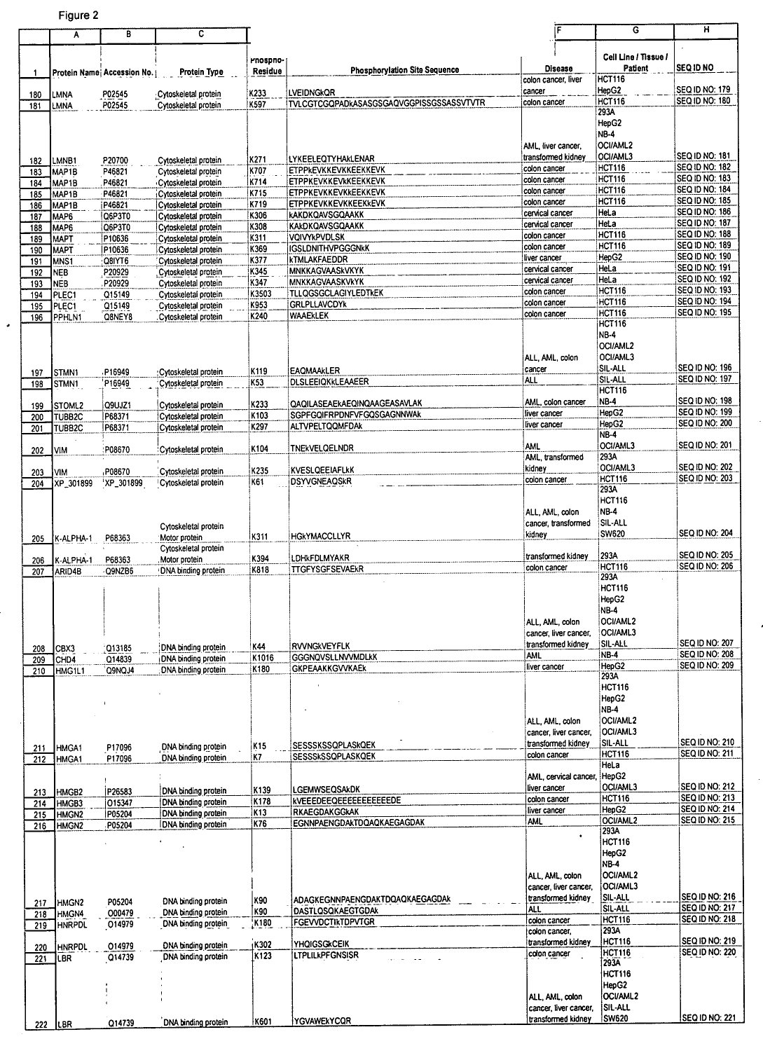

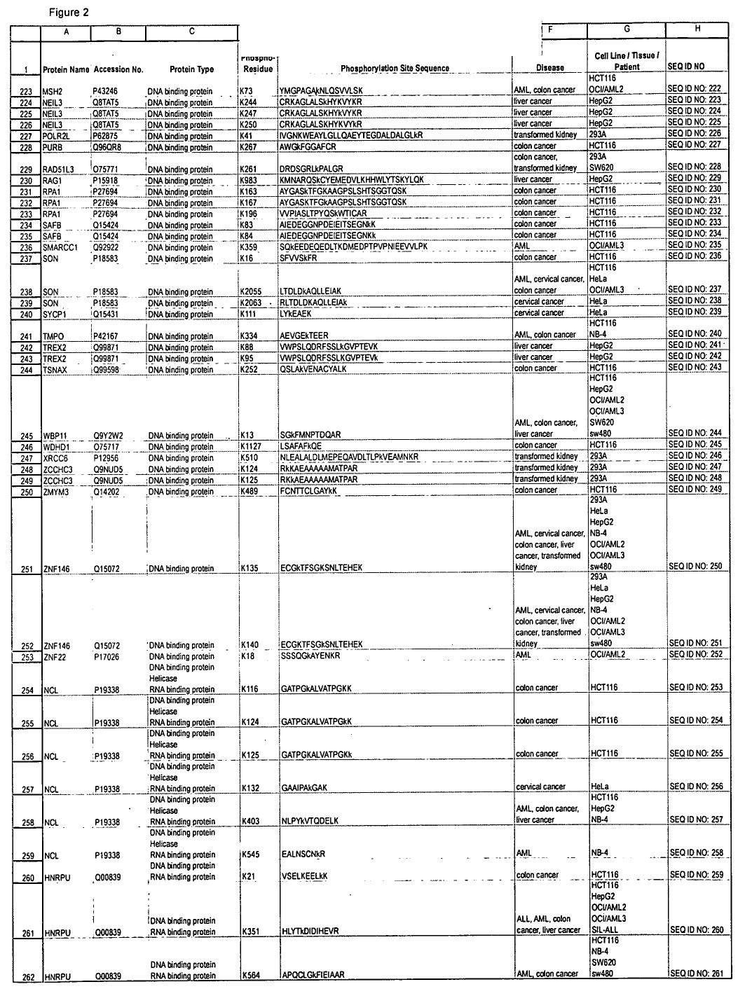

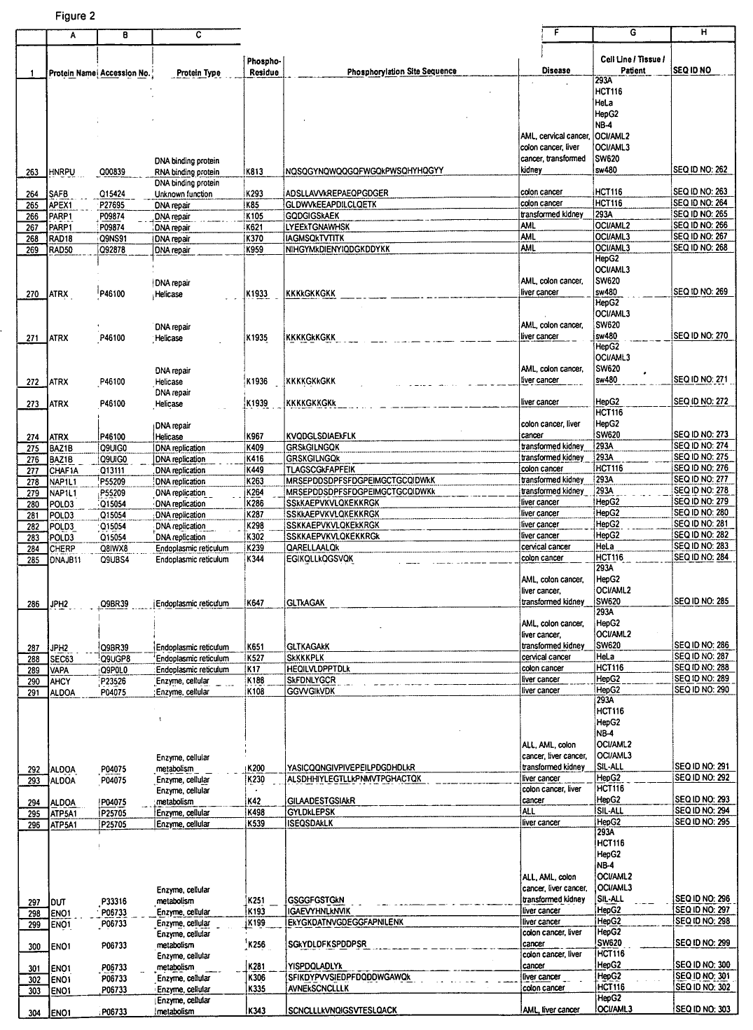

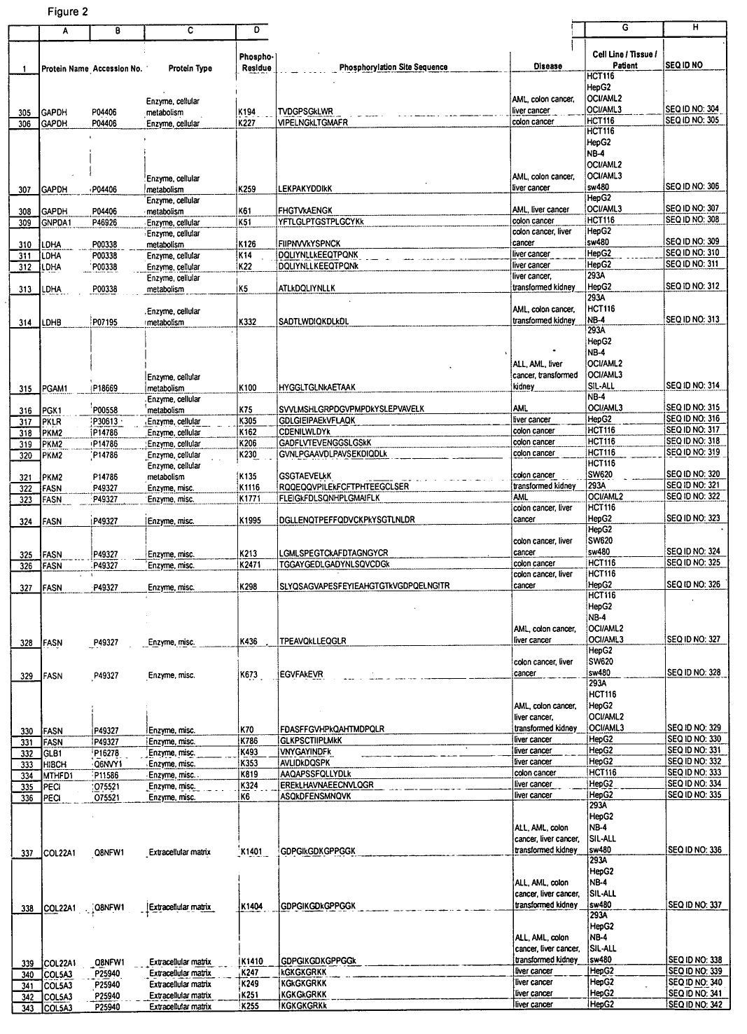

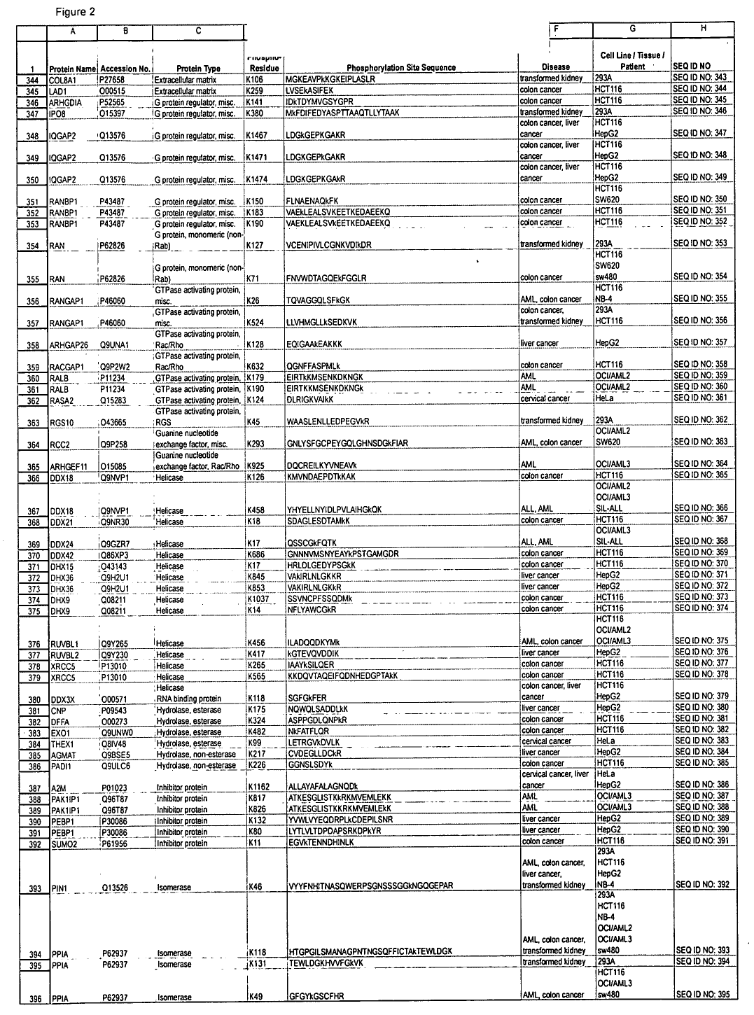

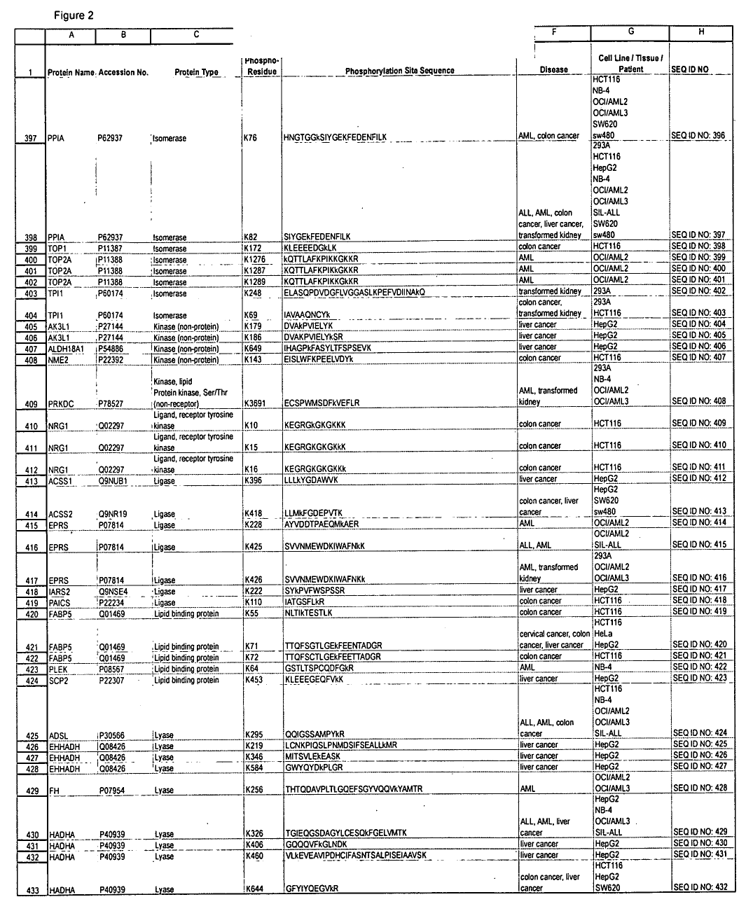

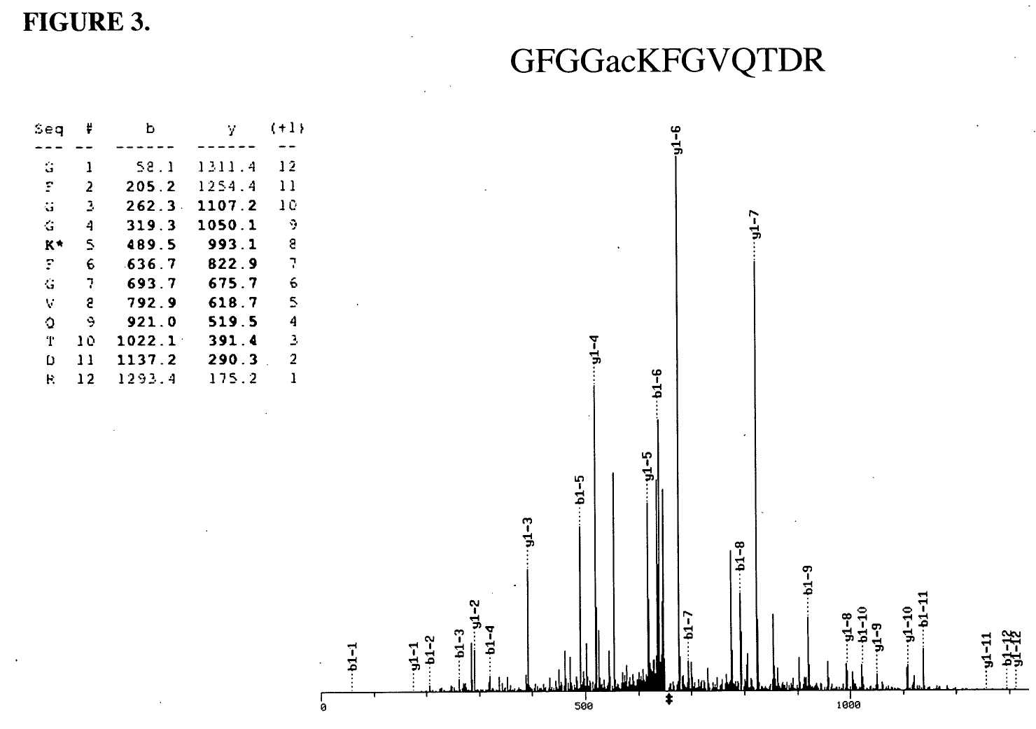

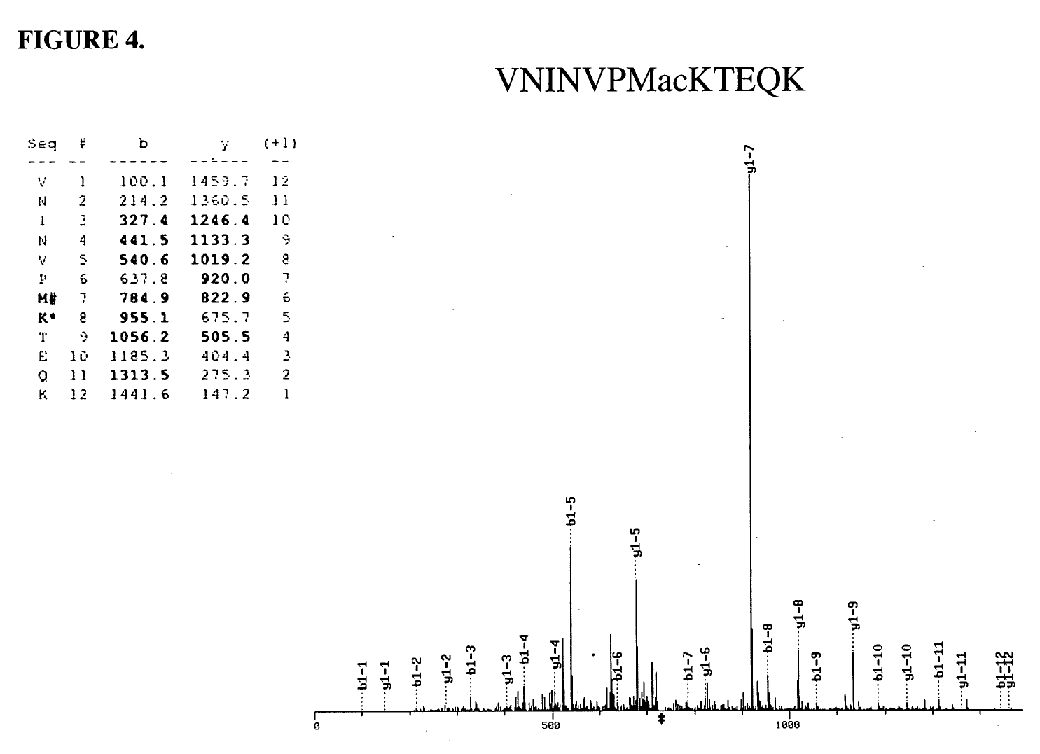

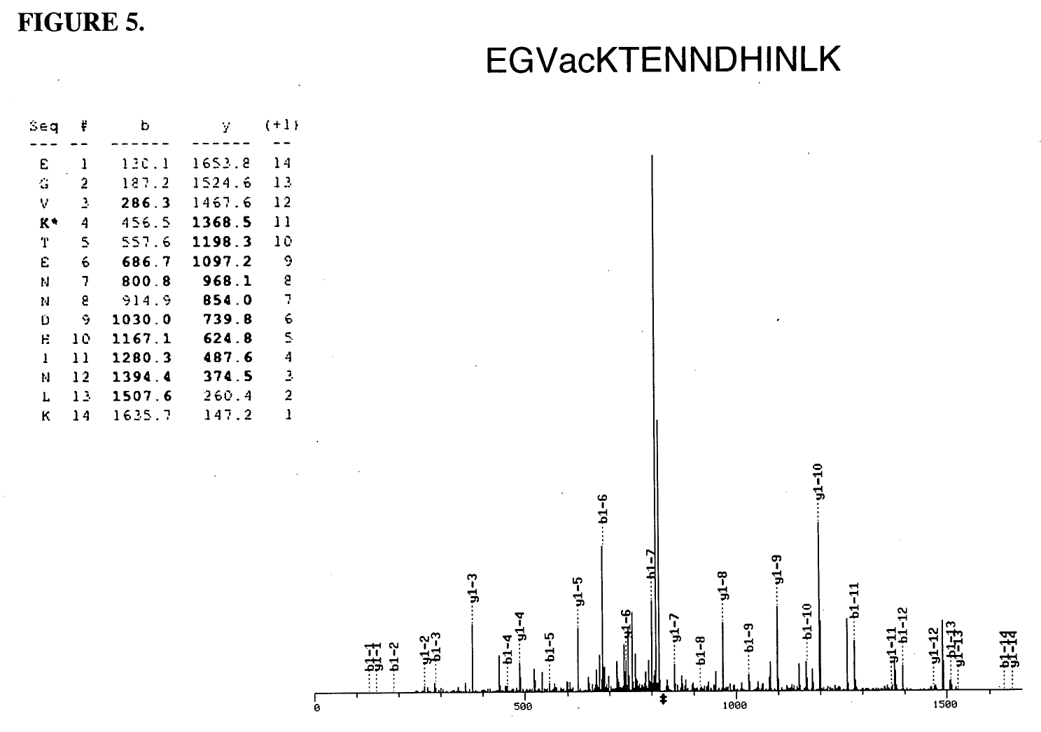

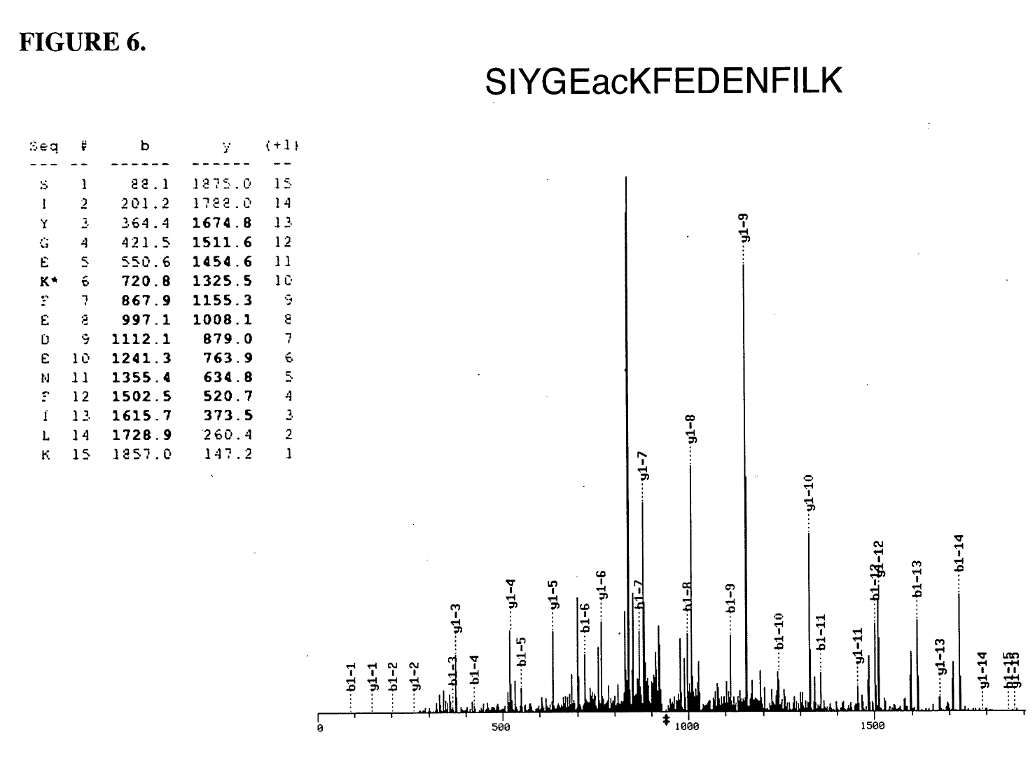

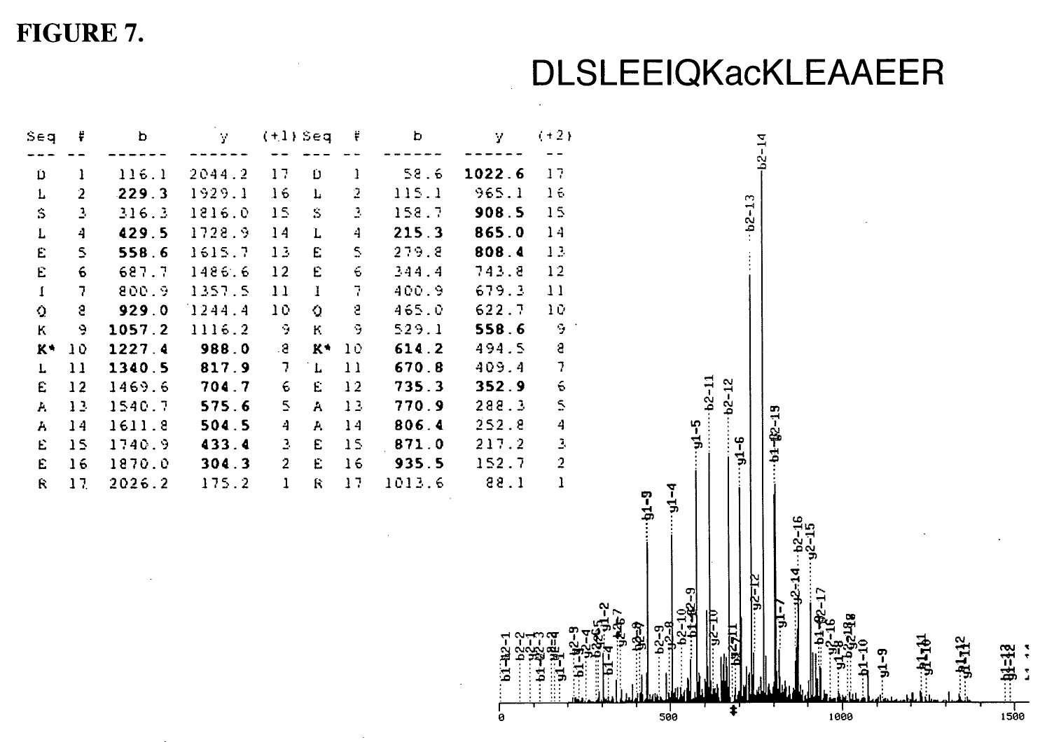

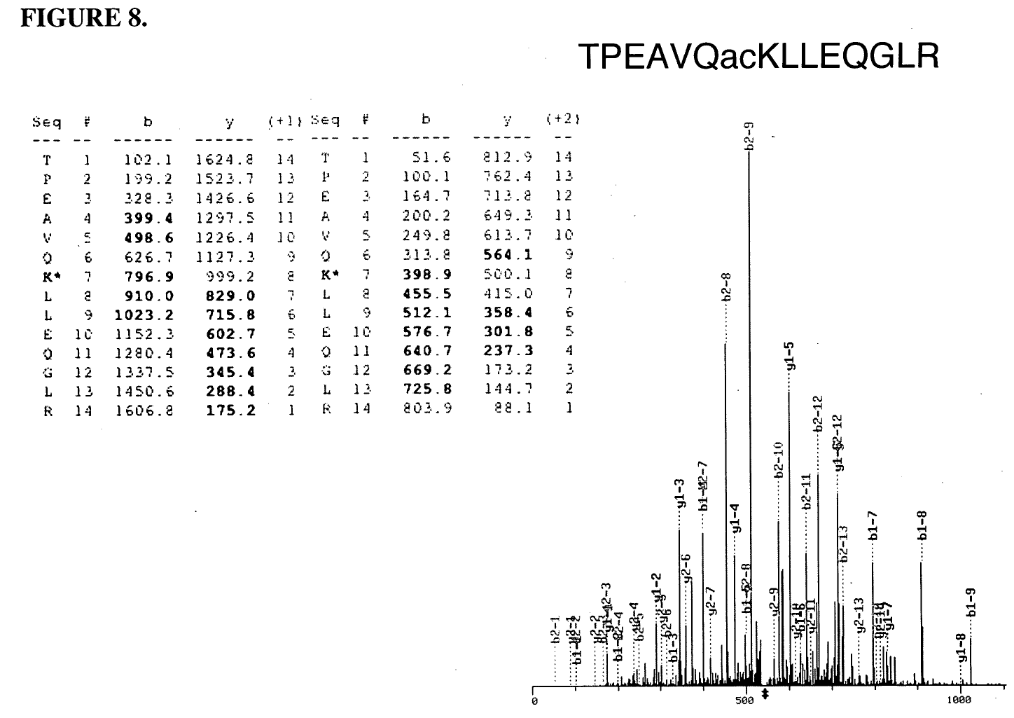

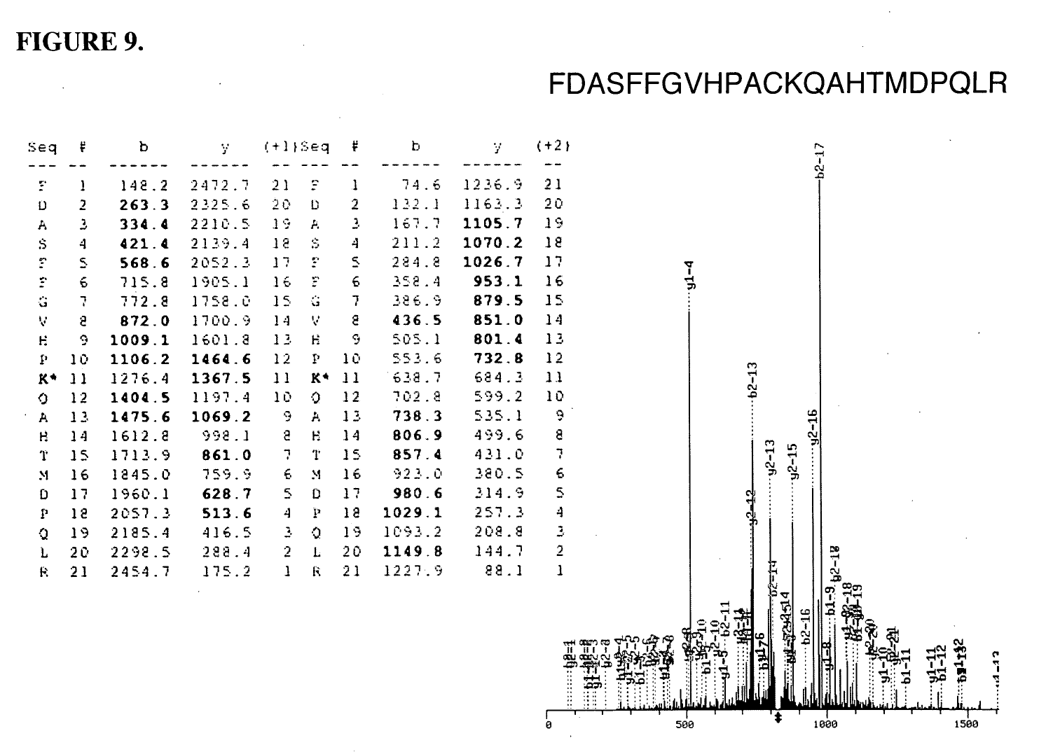

In accordance with the present invention, 432 novel protein acetylation sites underlying protein acetylation signaling pathways have now been discovered. These newly described acetylation sites were identified by employing the techniques described in “Immunoaffinity Isolation of Modified Peptides From Complex Mixtures,” U.S. Patent Publication No. 20030044848, Rush et al., using cellular extracts from a variety of human Cancer derived cell lines, e.g. HepG2, sw480 etc., as further described below. The novel acetylation sites (lysine), and their corresponding parent proteins, disclosed herein are listed in Table 1. These acetylation sites correspond to numerous different parent proteins (the full sequences of which (human) are all publicly available in SwissProt database and their Accession numbers listed in Column B of Table 1/FIG. 2), each of which fall into discrete protein type groups, for example DNA repair proteins, Adaptor Scaffold Proteins, and Enzyme proteins, etc. (see Column C of Table 1), the acetylation of which is relevant to signal transduction activity underlying protein acetylation signaling, as disclosed herein.

The discovery of the 432 novel protein acetylation sites described herein enables the production, by standard methods, of new reagents, such as acetylation site-specific antibodies and AQUA peptides (heavy-isotope labeled peptides), capable of specifically detecting and/or quantifying these acetylated sites/proteins. Such reagents are highly useful, inter alia, for studying signal transduction events underlying the progression of cancer. Accordingly, the invention provides novel reagents—acetyl-specific antibodies and AQUA peptides—for the specific detection and/or quantification of as protein acetylation signaling protein/polypeptide only when acetylated (or only when not acetylated) at a particular acetylation site disclosed herein. The invention also provides methods of detecting and/or quantifying one or more acetylated protein acetylation signaling proteins using the acetylation-site specific antibodies and AQUA peptides of the invention.

In part, the invention provides an isolated acetylation site-specific antibody that specifically binds a given protein acetylation signaling protein only when acetylated (or not acetylated, respectively) at a particular lysine enumerated in Column D of Table 1/FIG. 2 comprised within the acetylatable peptide site sequence enumerated in corresponding Column E. In further part, the invention provides a heavy-isotope labeled peptide (AQUA peptide) for the detection and quantification of a given Protein acetylation signaling protein, the labeled peptide comprising a particular acetylatable peptide site/sequence enumerated in Column E of Table 1/FIG. 2 herein. For example, among the reagents provided by the invention is an isolated acetylation site-specific antibody that specifically binds the VASP acetyltransferase only when acetylated (or only when not acetylated) at lysine 283 (see Row 8 (and Columns D and E) of Table 1/FIG. 2). By way of further example, among the group of reagents provided by the invention is an AQUA peptide for the quantification of acetylated VASP acetyltransferase protein, the AQUA peptide comprising the acetylatable peptide sequence listed in Column E, Row 8, of Table 1/FIG. 2 (which encompasses the acetylatable lysine at position 283).

In one embodiment, the invention provides an isolated acetylation site-specific antibody that specifically binds a human protein acetylation signaling protein selected from Column A of Table 1 (Rows 2-433) only when acetylated at the lysine residue listed in corresponding Column D of Table 1, comprised within the acetylatable peptide sequence listed in corresponding Column E of Table 1 (SEQ ID NOs: 1-432), wherein said antibody does not bind said signaling protein when not acetylated at said lysine. In another embodiment, the invention provides an isolated acetylation site-specific antibody that specifically binds a protein acetylation signaling protein selected from Column A of Table 1 only when not acetylated at the lysine residue listed in corresponding Column D of Table 1, comprised within the peptide sequence listed in corresponding Column E of Table 1 (SEQ ID NOs: 1-432), wherein said antibody does not bind said signaling protein when acetylated at said lysine. Such reagents enable the specific detection of acetylation (or non-acetylation) of a novel acetylatable site disclosed herein. The invention further provides immortalized cell lines producing such antibodies. In one preferred embodiment, the immortalized cell line is a rabbit or mouse hybridoma.

In another embodiment, the invention provides a heavy-isotope labeled peptide (AQUA peptide) for the quantification of an protein acetylation signaling protein selected from Column A of Table 1, said labeled peptide comprising the acetylatable peptide sequence listed in corresponding Column E of Table 1 (SEQ ID NOs: 1-432), which sequence comprises the acetylatable lysine listed in corresponding Column D of Table 1. In certain preferred embodiments, the acetylatable lysine within the labeled peptide is acetylated, while in other preferred embodiments, the acetylatable residue within the labeled peptide is not acetylated.

Reagents (antibodies and AQUA peptides) provided by the invention may conveniently be grouped by the type of protein acetylation signaling protein in which a given acetylation site (for which reagents are provided) occurs. The protein types for each respective protein (in which an acetylation site has been discovered) are provided in Column C of Table 1/FIG. 2, and include: Acetyltransferases, Adaptor/Scaffold proteins, Actin binding proteins, Adhesion proteins, Apoptosis proteins, Calcium-binding proteins, Cell Cycle Regulation proteins, Cell Surface proteins, DNA binding proteins, DNA replication proteins, Channel proteins, Chaperone proteins, Cellular Metabolism enzymes, Cytoskeletal proteins, DNA repair proteins, Endoplasmic reticulum proteins, Enzyme proteins, G protein and GTPase Activating proteins, Guanine Nucleotide Exchange Factors, Helicase proteins, Isomerase proteins, Extracelluar matrix proteins, Hydrolases, Ligase proteins, Lipid kinases, Inhibtor proteinsLipid Binding proteins and Lyases. Each of these distinct protein groups is considered a preferred subset of Protein acetylation signal transduction protein acetylation sites disclosed herein, and reagents for their detection/quantification may be considered a preferred subset of reagents provided by the invention.

Particularly preferred subsets of the acetylation sites (and their corresponding proteins) disclosed herein are those occurring on the following protein types/groups listed in Column C of Table 1/FIG. 2, DNA binding proteins, Acetyltransferases, DNA repair proteins, G protein/GTPase Activating proteins/Guanine Nucleotide Exchange Factors, Helicases, Chaperone proteins, Adaptor/Scaffold proteins, Cell cycle regulation proteins, Cytoskeletal proteins, Enzyme proteins, Isomerases and Actin binding proteins. Accordingly, among preferred subsets of reagents provided by the invention are isolated antibodies and AQUA peptides useful for the detection and/or quantification of the foregoing preferred protein/acetylation site subsets.

In one subset of preferred embodiments, there is provided:

(i) An isolated acetylation site-specific antibody that specifically binds an DNA repair protein selected from Column A, Rows 265-274, of Table 1 only when acetylated at the lysine listed in corresponding Column D, Rows 265-274, of Table 1, comprised within the acetylatable peptide sequence listed in corresponding Column E, Rows 265-274, of Table 1 (SEQ ID NOs: 264-273), wherein said antibody does not bind said protein when not acetylated at said lysine.
(ii) An equivalent antibody to (i) above that only binds the DNA repair protein when not acetylated at the disclosed site (and does not bind the protein when it is acetylated at the site).
(iii) A heavy-isotope labeled peptide (AQUA peptide) for the quantification of an DNA repair protein selected from Column A, Rows 265-274, said labeled peptide comprising the acetylatable peptide sequence listed in corresponding Column E, Rows 265-274, of Table 1 (SEQ ID NOs: 264-273), which sequence comprises the acetylatable lysine listed in corresponding Column D, Rows 265-274, of Table 1.

Among this preferred subset of reagents, antibodies and AQUA peptides for the detection/quantification of the following DNA repair protein acetylation sites is particularly preferred: PARP (K105) (see SEQ ID NO: 104).

In a second subset of preferred embodiments there is provided:

(i) An isolated acetylation site-specific antibody that specifically binds an Acetyltransferase selected from Column A, Rows 2-9, of Table 1 only when acetylated at the lysine listed in corresponding Column D, Rows 2-9, of Table 1, comprised within the acetylatable peptide sequence listed in corresponding Column E, Rows 2-9, of Table 1 (SEQ ID NOs: 1-8), wherein said antibody does not bind said protein when not acetylated at said lysine.
(ii) An equivalent antibody to (i) above that only binds the Acetyltransferase when not acetylated at the disclosed site (and does not bind the protein when it is acetylated at the site).
(iii) A heavy-isotope labeled peptide (AQUA peptide) for the quantification of an Acetyltransferase selected from Column A, Rows 2-9, said labeled peptide comprising the acetylatable peptide sequence listed in corresponding Column E, Rows 2-9, of Table 1 (SEQ ID NOs: 1-8), which sequence comprises the acetylatable lysine listed in corresponding Column D, Rows 2-9, of Table 1.

Among this preferred subset of reagents, antibodies and AQUA peptides for the detection/quantification of the following Acetyltransferase acetylation sites are particularly preferred: TAF1 (K705) (see SEQ ID NO: 8).

In another subset of preferred embodiments there is provided:

(i) An isolated acetylation site-specific antibody that specifically binds a Chaperone Protein selected from Column A, Rows 117-154, of Table 1 only when acetylated at the lysine listed in corresponding Column D, Rows 117-154, of Table 1, comprised within the acetylatable peptide sequence listed in corresponding Column E, Rows 117-154, of Table 1 (SEQ ID NOs: 116-153), wherein said antibody does not bind said protein when not acetylated at said lysine.
(ii) An equivalent antibody to (i) above that only binds the Chaperone Protein when not acetylated at the disclosed site (and does not bind the protein when it is acetylated at the site).
(iii) A heavy-isotope labeled peptide (AQUA peptide) for the quantification of a Chaperone Protein selected from Column A, Rows 117-154, said labeled peptide comprising the acetylatable peptide sequence listed in corresponding Column E, Rows 117-154, of Table 1 (SEQ ID NOs: 116-153), which sequence comprises the acetylatable lysine listed in corresponding Column D, Rows 117-154, of Table 1.

Among this preferred subset of reagents, antibodies and AQUA peptides for the detection/quantification of the following Chaperone Protein acetylation sites are particularly preferred: HSP90AA1 (K314) (see SEQ ID NO: 121).

In still another subset of preferred embodiments there is provided:

(i) An isolated acetylation site-specific antibody that specifically binds a Cytoskeletal protein selected from Column A, Rows 156-206, of Table 1 only when acetylated at the lysine listed in corresponding Column D, Rows 156-206, of Table 1, comprised within the acetylatable peptide sequence listed in corresponding Column E, Rows 156-206, of Table 1 (SEQ ID NOs: 155-205), wherein said antibody does not bind said protein when not acetylated at said lysine.
(ii) An equivalent antibody to (i) above that only binds the Cytoskeletal protein when not acetylated at the disclosed site (and does not bind the protein when it is acetylated at the site).
(iii) A heavy-isotope labeled peptide (AQUA peptide) for the quantification of a Cytoskeletal protein selected from Column A, Rows 156-206, said labeled peptide comprising the acetylatable peptide sequence listed in corresponding Column E, Rows 156-206, of Table 1 (SEQ ID NOs: 155-205), which sequence comprises the acetylatable lysine listed in corresponding Column D, Rows 156-206, of Table 1.

Among this preferred subset of reagents, antibodies and AQUA peptides for the detection/quantification of the following Cytoskeletal protein acetylation sites are particularly preferred: K-ALPHA-1 (K352) and STMN1 (K119) (see SEQ ID NO: 169 and 196).

In still another subset of preferred embodiments there is provided:

(i) An isolated acetylation site-specific antibody that specifically binds a G protein/GTPase/Guanine nucleotide exchange factor selected from Column A, Rows 346-365, of Table 1 only when acetylated at the lysine listed in corresponding Column D, Rows 346-365, of Table 1, comprised within the acetylatable peptide sequence listed in corresponding Column E, Rows 346-365, of Table 1 (SEQ ID NOs: 345-364), wherein said antibody does not bind said protein when not acetylated at said lysine.
(ii) An equivalent antibody to (i) above that only binds the G protein/GTPase/Guanine nucleotide exchange factor when not acetylated at the disclosed site (and does not bind the protein when it is acetylated at the site).
(iii) A heavy-isotope labeled peptide (AQUA peptide) for the quantification of a G protein/GTPase/Guanine nucleotide exchange factor selected from Column A, Rows 346-365, said labeled peptide comprising the acetylatable peptide sequence listed in corresponding Column E, Rows 346-365, of Table 1 (SEQ ID NOs: 345-364), which sequence comprises the acetylatable lysine listed in corresponding Column D, Rows 346-365, of Table 1.

Among this preferred subset of reagents, antibodies and AQUA peptides for the detection/quantification of the following G protein/GTPase/Guanine nucleotide exchange factor acetylation sites are particularly preferred: RALB (K179) (see SEQ ID NO: 359).

In still another subset of preferred embodiments there is provided:

(i) An isolated acetylation site-specific antibody that specifically binds an Enzyme protein selected from Column A, Rows 290-336, of Table 1 only when acetylated at the lysine listed in corresponding Column D, Rows 290-336, of Table 1, comprised within the acetylatable peptide sequence listed in corresponding Column E, Rows 290-336 of Table 1 (SEQ ID NOs: 289-335), wherein said antibody does not bind said protein when not acetylated at said lysine.
(ii) An equivalent antibody to (i) above that only binds the Enzyme protein when not acetylated at the disclosed site (and does not bind the protein when it is acetylated at the site).
(iii) A heavy-isotope labeled peptide (AQUA peptide) for the quantification of an Enzyme protein selected from Column A, Rows 290-336, said labeled peptide comprising the acetylatable peptide sequence listed in corresponding Column E, Rows 290-336, of Table 1 (SEQ ID NOs: 289-335), which sequence comprises the acetylatable lysine listed in corresponding Column D, Rows 290-336, of Table 1.

Among this preferred subset of reagents, antibodies and AQUA peptides for the detection/quantification of the following Enzyme protein acetylation sites are particularly preferred: GNPDA (K51), PKM2 (K162) and FASN (K1116) (see SEQ ID NO: 308, 317 and 321).

In yet another subset of preferred embodiments, there is provided:

(i) An isolated acetylation site-specific antibody that specifically binds a DNA binding protein selected from Column A, Rows 207-264, of Table 1 only when acetylated at the lysine listed in corresponding Column D, Rows 207-264, of Table 1, comprised within the acetylatable peptide sequence listed in corresponding Column E, Rows 207-264, of Table 1 (SEQ ID NOs: 206-263), wherein said antibody does not bind said protein when not acetylated at said lysine.
(ii) An equivalent antibody to (i) above that only binds the DNA binding protein when not acetylated at the disclosed site (and does not bind the protein when it is acetylated at the site).
(iii) A heavy-isotope labeled peptide (AQUA peptide) for the quantification of a DNA binding protein that is a DNA binding protein selected from Column A, Rows 207-264, said labeled peptide comprising the acetylatable peptide sequence listed in corresponding Column E, Rows 207-264, of Table 1 (SEQ ID NOs: 206-263), which sequence comprises the acetylatable lysine listed in corresponding Column D, Rows 207-264, of Table 1.

Among this preferred subset of reagents, antibodies and AQUA peptides for the detection/quantification of the following DNA binding protein acetylation sites are particularly preferred: MSH2 (K73) (see SEQ ID NO: 222)

In yet another subset of preferred embodiments, there is provided:

(i) An isolated acetylation site-specific antibody specifically binds an Isomerase selected from Column A, Rows 393-404, of Table 1 only when acetylated at the lysine listed in corresponding Column D, Rows 393-404, of Table 1, comprised within the acetylatable peptide sequence listed in corresponding Column E, Rows 393-404, of Table 1 (SEQ ID NOs: 392-403), wherein said antibody does not bind said protein when not acetylated at said lysine.
(ii) An equivalent antibody to (i) above that only binds the Isomerase when not acetylated at the disclosed site (and does not bind the protein when it is acetylated at the site).
(iii) A heavy-isotope labeled peptide (AQUA peptide) for the quantification of an Isomerase selected from Column A, Rows 393-404, said labeled peptide comprising the acetylatable peptide sequence listed in corresponding Column E, Rows 393-404, of Table 1 (SEQ ID NOs: 392-403), which sequence comprises the acetylatable lysine listed in corresponding Column D, Rows 393-404, of Table 1.

Among this preferred subset of reagents, antibodies and AQUA peptides for the detection/quantification of the following Isomerase acetylation sites are particularly preferred: PIN1 (K46) (see SEQ ID NO: 392).

In yet another subset of preferred embodiments, there is provided:

(i) An isolated acetylation site-specific antibody that specifically binds an Adaptor/Scaffold protein selected from Column A, Rows 23-56, of Table 1 only when acetylated at the lysine listed in corresponding Column D, Rows 23-56, of Table 1, comprised within the acetylatable peptide sequence listed in corresponding Column E, Rows 23-56, of Table 1 (SEQ ID NOs: 22-55), wherein said antibody does not bind said protein when not acetylated at said lysine.
(ii) An equivalent antibody to (i) above that only binds the Adaptor/Scaffold protein when not acetylated at the disclosed site (and does not bind the protein when it is acetylated at the site).
(iii) A heavy-isotope labeled peptide (AQUA peptide) for the quantification of a Adaptor/Scaffold protein selected from Column A, Rows 23-56, said labeled peptide comprising the acetylatable peptide sequence listed in corresponding Column E, Rows 23-56, of Table 1 (SEQ ID NOs: 22-55), which sequence comprises the acetylatable lysine and lysine listed in corresponding Column D, Rows 23-56, of Table 1.

Among this preferred subset of reagents, antibodies and AQUA peptides for the detection/quantification of the following Adaptor/Scaffold protein acetylation sites are particularly preferred: GNB2L1 (K172) (see SEQ ID NO: 35).

In still another subset of preferred embodiments, there is provided:

(i) An isolated acetylation site-specific antibody that specifically binds a Cell cycle regulation protein selected from Column A, Rows 81-98, of Table 1 only when acetylated at the lysine listed in corresponding Column D, Rows 81-98, of Table 1, comprised within the acetylatable peptide sequence listed in corresponding Column E, Rows 81-98, of Table 1 (SEQ ID NOs: 80-97), wherein said antibody does not bind said protein when not acetylated at said lysine.
(ii) An equivalent antibody to (i) above that only binds the Cell cycle regulation protein when not acetylated at the disclosed site (and does not bind the protein when it is acetylated at the site).
(iii) A heavy-isotope labeled peptide (AQUA peptide) for the quantification of a Cell cycle regulation protein selected from Column A, Rows 81-98, said labeled peptide comprising the acetylatable peptide sequence listed in corresponding Column E, Rows 81-98, of Table 1 (SEQ ID NOs: 80-97), which sequence comprises the acetylatable lysine listed in corresponding Column D, Rows 81-98, of Table 1.

Among this preferred subset of reagents, antibodies and AQUA peptides for the detection/quantification of the following Cell cycle regulation protein acetylation sites are particularly preferred: CULL (K689) and PCNA (K80) (see SEQ ID NOs: 81 and 89).

In still another subset of preferred embodiments, there is provided:

(i) An isolated acetylation site-specific antibody that specifically binds an Actin binding protein selected from Column A, Rows 10-22, of Table 1 only when acetylated at the lysine listed in corresponding Column D, Rows 10-22, of Table 1, comprised within the acetylatable peptide sequence listed in corresponding Column E, Rows 10-22, of Table 1 (SEQ ID NOs: 9-21), wherein said antibody does not bind said protein when not acetylated at said lysine.
(ii) An equivalent antibody to (i) above that only binds the Actin binding protein when not acetylated at the disclosed site (and does not bind the protein when it is acetylated at the site).
(iii) A heavy-isotope labeled peptide (AQUA peptide) for the quantification of an Actin binding protein selected from Column A, Rows 10-22, said labeled peptide comprising the acetylatable peptide sequence listed in corresponding Column E, Rows 10-22, of Table 1 (SEQ ID NOs: 9-21), which sequence comprises the acetylatable lysine listed in corresponding Column D, Rows 10-22, of Table 1.

Among this preferred subset of reagents, antibodies and AQUA peptides for the detection/quantification of the following Actin binding protein acetylation sites are particularly preferred: CTTN (K198) (see SEQ ID NOs: 17).

In still another subset of preferred embodiments, there is provided:

(i) An isolated acetylation site-specific antibody that specifically binds a Helicase selected from Column A, Rows 366-380, of Table 1 only when acetylated at the lysine listed in corresponding Column D, Rows 366-380, of Table 1, comprised within the acetylatable peptide sequence listed in corresponding Column E, Rows 366-380, of Table 1 (SEQ ID NOs: 365-379), wherein said antibody does not bind said protein when not acetylated at said lysine.
(ii) An equivalent antibody to (i) above that only binds the Helicase when not acetylated at the disclosed site (and does not bind the protein when it is acetylated at the site).
(iii) A heavy-isotope labeled peptide (AQUA peptide) for the quantification of a Helicase selected from Column A, Rows 366-380, said labeled peptide comprising the acetylatable peptide sequence listed in corresponding Column E, Rows 366-380, of Table 1 (SEQ ID NOs: 365-379), which sequence comprises the acetylatable lysine listed in corresponding Column D, Rows 366-380, of Table 1.

Among this preferred subset of reagents, antibodies and AQUA peptides for the detection/quantification of the following Actin binding protein acetylation sites are particularly preferred: XRCC5 (K265) (see SEQ ID NO: 377).

The invention also provides, in part, an immortalized cell line producing an antibody of the invention, for example, a cell line producing an antibody within any of the foregoing preferred subsets of antibodies. In one preferred embodiment, the immortalized cell line is a rabbit hybridoma or a mouse hybridoma.

In certain other preferred embodiments, a heavy-isotope labeled peptide (AQUA peptide) of the invention (for example, an AQUA peptide within any of the foregoing preferred subsets of AQUA peptides) comprises a disclosed site sequence wherein the acetylatable lysine is acetylated. In certain other preferred embodiments, a heavy-isotope labeled peptide of the invention comprises a disclosed site sequence wherein the acetylatable lysine is not acetylated.

The foregoing subsets of preferred reagents of the invention should not be construed as limiting the scope of the invention, which, as noted above, includes reagents for the detection and/or quantification of disclosed acetylation sites on any of the other protein type/group subsets (each a preferred subset) listed in Column C of Table 1/FIG. 2.

Also provided by the invention are methods for detecting or quantifying a protein acetylation signaling protein that is lysine-acetylated, said method comprising the step of utilizing one or more of the above-described reagents of the invention to detect or quantify one or more protein acetylation signaling protein(s) selected from Column A of Table 1 only when acetylated at the lysine listed in corresponding Column D of Table 1. In certain preferred embodiments of the methods of the invention, the reagents comprise a subset of preferred reagents as described above.

The identification of the disclosed novel protein acetylation signaling sites, and the standard production and use of the reagents provided by the invention are described in further detail below and in the Examples that follow.

All cited references are hereby incorporated herein, in their entirety, by reference. The Examples are provided to further illustrate the invention, and do not in any way limit its scope, except as provided in the claims appended hereto.

TABLE 1
Newly Discovered Protein Acetylation Sites.

| | | | | | | |
|---|---|---|---|---|---|---|
| 1 | A | B | C | D | E | H |

| | | | | | | |
|---|---|---|---|---|---|---|
| 2 | ARD1A | P41227 | Acetyltransferase | K136 | AALHLYSNTLNFQISEVEPkYYADGEDAYA | SEQ ID NO:1 |
| | | | | | MKR | |
| 3 | CHAT | P28329 | Acetyltransferase | K16 | RGLGGGGkWKR | SEQ ID NO: 2 |
| 4 | CHAT | P28329 | Acetyltransferase | K18 | RGLGGGGKWkR | SEQ ID NO: 3 |
| 5 | GNPNAT1 | Q96EK6 | Acetyltransferase | K152 | KLNCYkITLECLPQNVGFYK | SEQ ID NO: 4 |
| 6 | MAK3P | Q9GZZ1 | Acetyltransferase | K34 | LNQVIFPVSYNDkFYK | SEQ ID NO: 5 |
| 7 | MAK3P | Q9GZZ1 | Acetyltransferase | K37 | FYkDVLEVGELAK | SEQ ID NO: 6 |
| 8 | VASP | P50552 | Acetyltransferase | K283 | RKATQVGEk | SEQ ID NO: 7 |
| | | | Cytoskeletal | | | |
| | | | protein | | | |
| 9 | TAF1 | P21675 | Acetyltransferase | K705 | NYYkR | SEQ ID NO: 8 |
| | | | Protein kinase | | | |
| | | | Protein kinase, | | | |
| | | | Ser/Thr (non- | | | |
| | | | receptor) | | | |
| | | | Transcription, | | | |
| | | | coactivator/ | | | |
| | | | corepressor | | | |
| 10 | CAPZA1 | P52907 | Actin binding | K19 | IAAkFITHAPPGEFNEVFNDVR | SEQ ID NO: 9 |
| | | | protein | | | |
| 11 | CAPZA1 | P52907 | Actin binding | K273 | TKIDWNkILSYK | SEQ ID NO: 10 |
| | | | protein | | | |
| 12 | CAPZB | P47756 | Actin binding | K235 | STLNEIYFGkTK | SEQ ID NO: 11 |
| | | | protein | | | |
| 13 | CORO1C | Q9ULV4 | Actin binding | K418 | NILDSKPTANkK | SEQ ID NO: 12 |
| | | | protein | | | |
| 14 | DSTN | P60981 | Actin binding | K112 | KEELMFFLWAPELAPLk | SEQ ID NO: 13 |
| | | | protein | | | |
| 15 | FLNA | P21333 | Actin binding | K179 | LLGWIQNkLPQLPITNFSR | SEQ ID NO: 14 |
| | | | protein | | | |
| 16 | PLS3 | P13797 | Actin binding | K332 | IDINMSGFNETDDLkR | SEQ ID NO: 15 |
| | | | protein | | | |
| 17 | CTTN | Q14247 | Actin binding | K124 | GFGGkFGVQMDR | SEQ ID NO: 16 |
| | | | protein | | | |
| | | | Cytoskeletal | | | |
| | | | protein | | | |
| 18 | CTTN | Q14247 | Actin binding | K198 | GFGGkYGIDKDKVDK | SEQ ID NO: 17 |
| | | | protein | | | |
| | | | Cytoskeletal | | | |
| | | | protein | | | |
| 19 | CTTN | Q14147 | Actin binding | K235 | GFGGkFGVQTDR | SEQ ID NO: 18 |
| | | | protein | | | |
| | | | Cytoskeletal | | | |
| | | | protein | | | |
| 20 | CTTN | Q14247 | Actin binding | K272 | TGFGGkFGVQSER | SEQ ID NO: 19 |
| | | | protein | | | |
| | | | Cytoskeletal | | | |
| | | | protein | | | |
| 21 | CTTN | Q14247 | Actin binding | K290 | QDSAAVGFDYkEK | SEQ ID NO: 20 |
| | | | protein | | | |
| | | | Cytoskeletal | | | |
| | | | protein | | | |
| 22 | CTTN | Q14247 | Actin binding | K309 | GFGGkYGVQK | SEQ ID NO: 21 |
| | | | protein | | | |
| | | | Cytoskeletal | | | |
| | | | protein | | | |
| 23 | AKAP12 | Q5SZ80 | Adaptor/scaffold | K614 | EGVTPWASFkK | SEQ ID NO: 22 |
| 24 | AKAP12 | Q5SZ80 | Adaptor/scaffold | K771 | KkSKSKLEEK | SEQ ID NO: 23 |
| 25 | AKAP12 | Q5SZ80 | Adaptor/scaffold | K775 | KKSKSkLEEK | SEQ ID NO: 24 |
| 26 | ALS2CR19 | Q8TEW8 | Adaptor/scaffold | K844 | kEKGKLKVKEKK | SEQ ID NO: 25 |
| 27 | ALS2CR19 | Q8TEW8 | Adaptor/scaffold | K848 | KEKGkLKVKEKK | SEQ ID NO: 26 |
| 28 | ALS2CR19 | Q8TEW8 | Adaptor/scaffold | K850 | KEKGKLkVKEKK | SEQ ID NO: 27 |
| 29 | ALS2CR19 | Q8TEW8 | Adaptor/scaffold | K854 | KEKGKLKVKEkK | SEQ ID NO: 28 |
| 30 | AP1GBP1 | Q9UMZ2 | Adaptor/scaffold | K513 | ALPSMDkYAVFK | SEQ ID NO: 29 |
| 31 | AP1GBP1 | Q9UMZ2 | Adaptor/scaffold | K744 | GGQNSTAASTkYDVFR | SEQ ID NO: 30 |
| 32 | CACYBP | Q9HB71 | Adaptor/scaffold | K207 | IYEDGDDDMkR | SEQ ID NO: 31 |
| 33 | CBX4 | O00257 | Adaptor/scaffold | K147 | SGkYYYQLNSK | SEQ ID NO: 32 |
| 34 | ENTH | Q14677 | Adaptor/scaffold | K625 | QDAFANFANFSk | SEQ ID NO: 33 |
| 35 | GKAP1 | Q5VSY0 | Adaptor/scaffold | K58 | KREkRRKKKEQQQSEANELRNLAFKK | SEQ ID NO: 34 |
| 36 | GNB2L1 | P63244 | Adaptor/scaffold | K172 | FSPNSSNPIIVSCGWDkLVK | SEQ ID NO: 35 |
| 37 | HGS | O14964 | Adaptor/scaffold | K494 | EkLRRAAEEAER | SEQ ID NO: 36 |
| 38 | MAGI2 | Q86UL8 | Adaptor/scaffold | K1426 | kAAVAPGPWK | SEQ ID NO: 37 |
| 39 | MRVI1 | Q9Y6F6 | Adaptor/scaffold | K405 | FAGkAGGKLAKAPGLK | SEQ ID NO: 38 |
| 40 | MRVI1 | Q9Y6F6 | Adaptor/scaffold | K409 | FAGKAGGkLAKAPGLK | SEQ ID NO: 39 |
| 41 | MRVI1 | Q9Y6F6 | Adaptor/scaffold | K412 | FAGKAGGKLAkAPGLK | SEQ ID NO: 40 |
| 42 | PPFIBP1 | Q86W92 | Adaptor/scaffold | K479 | SSSLGNLKKETSDGEk | SEQ ID NO: 41 |
| 43 | PRKCSH | P14314 | Adaptor/scaffold | K458 | LGGSPTSLGTWGSWIGPDHDkFSAMK | SEQ ID NO: 42 |
| 44 | PYCARD | Q9ULZ3 | Adaptor/scaffold | K21 | DAILDALENLTAEELkK | SEQ ID NO: 43 |
| 45 | RANBP2 | P49792 | Adaptor/scaffold | K1977 | GFSGAGEkLFSSQYGK | SEQ ID NO: 44 |
| 46 | SNCB | Q16143 | Adaptor/scaffold | K12 | MDVFMKGLSMAk | SEQ ID NO: 45 |
| 47 | SPSB1 | Q96BD6 | Adaptor/scaffold | K10 | MGQKVTGGIkWDMRDPTYRPLK | SEQ ID NO: 46 |
| 48 | SPSB1 | Q96BD6 | Adaptor/scaffold | K4 | MGQkVTGGIKTVDMRDPTYRPLK | SEQ ID NO: 47 |
| 49 | VEZT | Q6P1Q3 | Adaptor/scaffold | K14 | EWAIKQGILLkVAETIK | SEQ ID NO: 48 |
| 50 | WAC | Q9BTA9 | Adaptor/scaffold | K302 | LPTPTSSVPAQkTER | SEQ ID NO: 49 |
| 51 | YWHAB | P31946 | Adaptor/scaffold | K70 | VISSIEQkTER | SEQ ID NO: 50 |
| 52 | YWHAE | P42655 | Adaptor/scaffold | K142 | YLAEFATGNDRk | SEQ ID NO: 51 |
| 53 | YWHAZ | P63104 | Adaptor/scaffold | K138 | YLAEVAAGDDkK | SEQ ID NO: 52 |
| 54 | YWHAZ | P63104 | Adaptor/scaffold | K139 | YLAEVAAGDDKk | SEQ ID NO: 53 |
| 55 | YWHAZ | P63104 | Adaptor/scaffold | K68 | VVSSIEQkTEGAEK | SEQ ID NO: 54 |
| 56 | ST13 | P50502 | Adaptor/scaffold | K186 | AIEINPDSAQPYkWR | SEQ ID NO: 55 |
| | | | Unknown fundtion | | | |
| 57 | MARCKSL1 | P49006 | Adhesion | K106 | LSGLSFkR | SEQ ID NO: 56 |
| 58 | ZYX | Q15942 | Adhesion | K24 | PSPAISVSVSAPAFYAPQkK | SEQ ID NO: 57 |
| 59 | ZYX | Q15942 | Adhesion | K25 | PSPAISVSVSAPAFYAPQKk | SEQ ID NO: 58 |
| 60 | ZYX | Q15942 | Adhesion | K279 | FTPVASkFSPGAPGGSGSQPNQK | SEQ ID NO: 59 |
| 61 | DSP | P15924 | Adhesion | K803 | LTEEETVCLDLDkVEAYR | SEQ ID NO: 60 |
| | | | Cytoskeletal | | | |
| | | | protein | | | |
| 62 | VCL | P18206 | Adhesion | K496 | AAVHLEGkIEQAQR | SEQ ID NO: 61 |
| | | | Cytoskeletal | | | |
| | | | protein | | | |
| 63 | VCL | P18206 | Adhesion | K778 | EVENSEDPkFR | SEQ ID NO: 62 |
| | | | Cytoskeletal | | | |
| | | | protein | | | |
| 64 | RPSA | P08865 | Adhesion | K57 | TWEkLLLAAR | SEQ ID NO: 63 |
| | | | Receptor, | | | |
| | | | misc. | | | |
| 65 | CAT | P04040 | Apoptosis | K237 | FHYkTDQGIK | SEQ ID NO: 64 |
| 66 | EP400 | Q96L91 | Apoptosis | K345 | TAVPPGLSSLPLTSVGNTGMkK | SEQ ID NO: 65 |
| 67 | FAU | P62861 | Apoptosis | K51 | FVNWPTFGkK | SEQ ID NO: 66 |
| 68 | FAU | P62861 | Apoptosis | K52 | FVNWPTFGKk | SEQ ID NO: 67 |
| 69 | HSP90B1 | P14625 | Apoptosis | K586 | GYEVIYLTEPVDEYCIQALPEFDGkR | SEQ ID NO: 68 |
| 70 | HSP90B1 | P14625 | Apoptosis | K75 | SEkFAFQAEVNR | SEQ ID NO: 69 |
| 71 | DAXX | Q9UER7 | Apoptosis | K208 | RLQEkELDLSELDDPDSAYLQEAR | SEQ ID NO: 70 |
| | | | Transcription, | | | |
| | | | coactivator/ | | | |
| | | | corepressor | | | |
| 72 | ANXA11 | P50995 | Calcium-binding | K255 | DLIKDLkSELSGNFEK | SEQ ID NO: 71 |
| | | | protein | | | |
| 73 | STIM1 | Q13586 | Calcium-binding | K673 | KkFPLKIFKKPLKK | SEQ ID NO: 72 |
| | | | protein | | | |
| 74 | STIM1 | Q13586 | Calcium-binding | K677 | KKFPLkIFKKPLKK | SEQ ID NO: 73 |
| | | | protein | | | |
| 75 | STIM1 | Q13586 | Calcium-binding | K680 | KKFPLKIFkKPLKK | SEQ ID NO: 74 |
| | | | protein | | | |
| 76 | STIM1 | Q13586 | Calcium-binding | K684 | KKFPLKIFKKPLkK | SEQ ID NO: 75 |
| | | | protein | | | |
| 77 | ANXA1 | P04083 | Calcium-binding | K312 | SEIDMNDIkAFYQK | SEQ ID NO: 76 |
| | | | protein | | | |
| | | | Lipid binding | | | |
| | | | protein | | | |
| 78 | ANXA1 | P04083 | Calcium-binding | K97 | AAYLQETGKPLDETLkK | SEQ ID NO: 77 |
| | | | protein | | | |
| | | | Lipid binding | | | |
| | | | protein | | | |
| 79 | ANXA2 | P07355 | Calcium-binding | K266 | GDLENAFLNLVQCIQNkPLYFADR | SEQ ID NO: 78 |
| | | | protein | | | |
| | | | Lipid binding | | | |
| | | | protein | | | |
| 80 | ANXA6 | P08133 | Calcium-binding | K418 | DLMTDLkSEISGDLAR | SEQ ID NO: 79 |
| | | | protein | | | |
| | | | Lipid binding | | | |
| | | | protein | | | |
| 81 | ASPM | Q8IZT6 | Cell cycle | K1903 | EHQAALkIQSAFR | SEQ ID NO: 80 |
| | | | regulation | | | |
| 82 | CUL1 | Q13616 | Cell cycle | K689 | VNINVPMkTEQK | SEQ ID NO: 81 |
| | | | regulation | | | |
| 83 | CUL4B | Q7Z673 | Cell cycle | K172 | SSTTVSSFANSkPGSAK | SEQ ID NO: 82 |
| | | | regulation | | | |
| 84 | ICF45 | Q53G12 | Cell cycle | K263 | LPTEMEGkK | SEQ ID NO: 83 |
| | | | regulation | | | |
| 85 | MKI67 | P46013 | Cell cycle | K1165 | kLTPSAGKAMLTPKPAGGDEKDIKAFMGTP | SEQ ID NO: 84 |
| | | | regulation | | VQK | |
| 86 | MKI67 | P46013 | Cell cycle | K1178 | KLTPSAGKAMLTPkPAGGDEKDIKAFMGTP | SEQ ID NO: 85 |
| | | | regulation | | VQK | |
| 87 | MKI67 | P46103 | Cell cycle | K1185 | KLTPSAGKAMLTPKPAGGDEkDIKAFMGTP | SEQ ID NO: 86 |
| | | | regulation | | VQK | |
| 88 | MKI67 | P46013 | Cell cycle | K379 | ESVNLGkSEGFK | SEQ ID NO: 87 |
| | | | regulation | | | |
| 89 | NUT | Q86Y26 | Cell cycle | K336 | kAASKTRAPR | SEQ ID NO: 88 |
| | | | regulation | | | |
| 90 | PCNA | P12004 | Cell cycle | K80 | ILkCAGNEDIITLR | SEQ ID NO: 89 |
| | | | regulation | | | |
| 91 | PTMA | P06454 | Cell cycle | K103 | AAEDDEDDDVDTkK | SEQ ID NO: 90 |
| | | | regulation | | | |
| 92 | PTMA | P06454 | Cell cycle | K104 | AAEDDEDDDVDTKk | SEQ ID NO: 91 |
| | | | regulation | | | |
| 93 | PTMS | P20962 | Cell cycle | K15 | SVEAMELSAkDLK | SEQ ID NO: 92 |
| | | | regulation | | | |
| 94 | PTMS | P20962 | Cell cycle | K92 | AAEEEDEADPkR | SEQ ID NO: 93 |
| | | | regulation | | | |
| 95 | SMC2L1 | O95347 | Cell cycle | K958 | HLFGQPNSAYDFk | SEQ ID NO: 94 |
| | | | regulation | | | |
| 96 | SMC4L1 | Q9NTJ3 | Cell cycle | K363 | SNILSNEMkAK | SEQ ID NO: 95 |
| | | | regulation | | | |
| 97 | CSPG6 | Q9UQE7 | Cell cycle | K105 | RVIGAkKDQYFLDKK | SEQ ID NO: 96 |
| | | | regulation | | | |
| 98 | CSPG6 | Q9UQ37 | Cell cycle | K106 | RVIGAKkDQYFLDKK | SEQ ID NO: 97 |
| | | | regulation | | | |
| 99 | ERVWE1 | Q9UQF0 | Cell surface | K490 | LQMEPkMQSKTKIYR | SEQ ID NO: 98 |
| 100 | ERVWE1 | Q9UQF0 | Cell surface | K494 | LQMEPKMQSkTKIYR | SEQ ID NO: 99 |
| 101 | HBEGF | Q99075 | Cell surface | K103 | KKGKGLGkKRDPCLR | SEQ ID NO: 100 |
| 102 | HBEGF | Q99075 | Cell surface | K104 | KGKGLGKkR | SEQ ID NO: 101 |
| 103 | HBEGF | Q99075 | Cell surface | K96 | kKGKGLGKKR | SEQ ID NO: 102 |
| 104 | HBEGF | Q99075 | Cell surface | K97 | KkGKGLGKKRDPCLR | SEQ ID NO: 103 |
| 105 | HBEGF | Q99075 | Cell surface | K99 | KGkGLGKKR | SEQ ID NO: 104 |
| 106 | NOMO3 | P69849 | Cell surface | K170 | IQSTVTQPGGkFAFFK | SEQ ID NO: 105 |
| 107 | TRPM1 | O75560 | Channel, calcium | K360 | GGRGkGKGKK | SEQ ID NO: 106 |
| 108 | TRPM1 | O75560 | Channel, calcium | K362 | GGRGKGkGKK | SEQ ID NO: 107 |
| 109 | MCOLN3 | Q8TDD5 | Channel, cation | K59 | kPWKLAIQILKIAMVTIQLVLFGLSNQMVVAFK | SEQ ID NO: 108 |
| 110 | MCOLN3 | Q8TDD5 | Channel, cation | K69 | KPWKLAIQILkIAMVTIQLVLFGLSNQMVVAFK | SEQ ID NO: 109 |
| 111 | CLIC1 | O00299 | Channel, chloride | K119 | FSAYIkNSNPALNDNLEK | SEQ ID NO: 110 |
| 112 | GABRR3 | XP_116036 | Channel, ligand- | K55 | MKKDDSTkARPQK | SEQ ID NO: 111 |
| | | | gated | | | |
| 113 | IPR2 | Q14571 | Channel, ligand- | K613 | HITAkEIETFVSLLR | SEQ ID NO: 112 |
| | | | gated | | | |
| 114 | VDAC1 | P21796 | Channel, misc. | K224 | FGIAAkYQIDPDACFSAK | SEQ ID NO: 113 |
| 115 | VDAC1 | P21796 | Channel, misc. | K28 | GYGFGLIkLDLK | SEQ ID NO: 114 |
| 116 | VDAC2 | P45880 | Channel, misc. | K54 | GFGFGLVkLDVK | SEQ ID NO: 115 |
| 117 | AHSA1 | O95433 | Chaperone | K212 | ITLkETFLTSPEELYR | SEQ ID NO: 116 |
| 118 | CCT5 | P48843 | Chaperone | K275 | HKLDVTSVEDYk | SEQ ID NO: 117 |
| 119 | CCT8 | P50990 | Chaperone | K400 | AVDDGVNTFkVLTR | SEQ ID NO: 118 |
| 120 | CDC37 | Q16543 | Chaperone | K78 | ELEVAEGGkAELER | SEQ ID NO: 119 |
| 121 | HSP90AA1 | P07900 | Chaperone | K283 | EkYIDQEELNK | SEQ ID NO: 120 |
| 122 | HSP90AA1 | P07900 | Chaperone | K314 | NPDDITNEEYGEFYk | SEQ ID NO: 121 |
| 123 | HSP90AB1 | P08238 | Chaperone | K427 | CLELFSELAEDKENYkK | SEQ ID NO: 122 |
| 124 | HSP90AB1 | P08238 | Chaperone | K428 | CLELFSELAEDKENYKk | SEQ ID NO: 123 |
| 125 | HSP90AB1 | P08238 | Chaperone | K559 | AkFENLCK | SEQ ID NO: 124 |
| 126 | HSPA1B | P08107 | Chaperone | K246 | LVNHFVEEFkR | SEQ ID NO: 125 |
| 127 | HSPA1B | P08107 | Chaperone | K71 | NQVALNPQNTVFDAk. | SEQ ID NO: 126 |
| 128 | HSPA1B | P08107 | Chaperone | K88 | FGDPVVQSDMk | SEQ ID NO: 127 |
| 129 | HSPA5 | P11021 | Chaperone | K113 | TWNDPSVQQDIk | SEQ ID NO: 128 |
| 130 | HSPA5 | P11021 | Chaperone | K617 | AVEEKIEWLESHQDADIEDFkAK | SEQ ID NO: 129 |
| 131 | HSPA5 | P11021 | Chaperone | K651 | LYGSAGPPPTGEEDTAEkDEL | SEQ ID NO: 130 |
| 132 | HSPA5 | P11021 | Chaperone | K96 | NQLTSNPENTVFDAk | SEQ ID NO: 131 |
| 133 | HSPA8 | P11142 | Chaperone | K246 | MVNHFIAEFkR | SEQ ID NO: 132 |
| 134 | HSPA8 | P11142 | Chaperone | K88 | RFDDAVVQSDMk | SEQ ID NO: 133 |
| 135 | HSPA9B | P38646 | Chaperone | K135 | YDDPEVQkDIK | SEQ ID NO: 134 |
| 136 | HSPA9B | P38846 | Chaperone | K300 | ETGVDLTkDNMALQR | SEQ ID NO: 135 |
| 137 | HSPA9B | P38646 | Chaperone | K610 | MEEFKDQLPADECNkLKEEISK | SEQ ID NO: 136 |
| 138 | HSPB1 | P04792 | Chaperone | K123 | DGVVEITGkHEER | SEQ ID NO: 137 |
| 139 | HSPD1 | P10809 | Chaperone | K218 | TLNDELEIIEGMkFDR | SEQ ID NO: 138 |
| 140 | HSPD1 | P10809 | Chaperone | K233 | GYISPYFINTSkGQK | SEQ ID NO: 139 |
| 141 | HSPD1 | P10809 | Chaperone | K236 | GQkCEFQDAYVLLSEK | SEQ ID NO: 140 |
| 142 | HSPD1 | P10809 | Chaperone | K249 | CEFQDAYVLLSEkK | SEQ ID NO: 141 |
| 143 | HSPD1 | P10809 | Chaperone | K389 | IQEIIEQLDVTTSEYEKEkLNER | SEQ ID NO: 142 |
| 144 | HSPD1 | P10809 | Chaperone | K462 | CIPALDSLTPANEDQk | SEQ ID NO: 143 |
| 145 | HSPD1 | P10809 | Chaperone | K473 | TLkIPAMTIAK | SEQ ID NO: 144 |
| 146 | HSPD1 | P10809 | Chaperone | K87 | SIDLkDKYK | SEQ ID NO: 145 |
| 147 | NAP1L4 | Q99733 | Chaperone | K255 | MKSEPDKADPFSFEGPEIVDCDGCTIDWk | SEQ ID NO: 146 |
| 148 | NAP1L4 | Q99733 | Chaperone | K256 | MKSEPDKADPFSFEGPEIVDCDGCTIDWKk | SEQ ID NO: 147 |
| 149 | PDIA4 | P13667 | Chaperone | K484 | DLGLSESGEDVNAAILDESGkK | SEQ ID NO: 148 |
| 150 | PPIB | P23284 | Chaperone | K201 | IEVEkPFAIAKE | SEQ ID NO: 149 |
| 151 | STIP1 | P31948 | Chaperone | K312 | IGNSYFkEEK | SEQ ID NO: 150 |
| 152 | TCP1 | P17987 | Chaperone | K400 | SLHDALCVVkR | SEQ ID NO: 151 |
| 153 | PTGES3 | Q15185 | Chaperone | K48 | LTFSCLGGSDNFk | SEQ ID NO: 152 |
| | | | Enzyme, | | | |
| | | | cellular | | | |
| | | | metabolism | | | |
| 154 | PDIA3 | P30101 | Chaperone | K94 | VDCTANTNTCNkYGVSGYPTLK | SEQ ID NO: 153 |
| | | | Enzyme, misc. | | | |
| 155 | MIF | P14174 | Cytokine | K78 | SYSkLLCGLLAER | SEQ ID NO: 154 |
| 156 | ACTA1 | P68133 | Cytoskeletal | K86 | YPIEHGIITNWDDMEk | SEQ ID NO: 155 |
| | | | protein | | | |
| 157 | ACTB | P60709 | Cytoskeletal | K50 | HQGVMVGMGQkDSWGDEAQSK | SEQ ID NO: 156 |
| | | | protein | | | |
| 158 | ACTB | P60709 | Cytoskeletal | K61 | DSYVGDEAQSk | SEQ ID NO: 157 |
| | | | protein | | | |
| 159 | ACTN4 | O43707 | Cytoskeletal | K899 | MAPYQGPDAVPGALDYk | SEQ ID NO: 158 |
| | | | protein | | | |
| 160 | BSN | Q9UPA5 | Cytoskeletal | K2970 | KQAELDEEEkEIDAKLK | SEQ ID NO: 159 |
| | | | protein | | | |
| 161 | CFL1 | P23528 | Cytoskeletal | K126 | kKLTGIKHELQANCYEEVKDR | SEQ ID NO: 160 |
| | | | protein | | | |
| 162 | CFL1 | P23528 | Cytoskeletal | K127 | KkLTGIKHELQANCYEEVKDR | SEQ ID NO: 161 |
| | | | protein | | | |
| 163 | CFL1 | P23528 | Cytoskeletal | K132 | LTGIkHELQANCYEEVKDR | SEQ ID NO: 162 |
| | | | protein | | | |
| 164 | CFL1 | P23528 | Cytoskeletal | K144 | LTGIKHELQANCYEEVkDR | SEQ ID NO: 163 |
| | | | protein | | | |
| 165 | CNN3 | Q15417 | Cytoskeletal | K23 | IASkYDHQAEEDLR | SEQ ID NO: 164 |
| | | | protein | | | |
| 166 | DMD | P11532 | Cytoskeletal | K1017 | kYQSEFEEIEGR | SEQ ID NO: 165 |
| | | | protein | | | |
| 167 | DYNC1L11 | Q9Y6G9 | Cytosketetal | K428 | SVSSNVASVSPIPAGSkK | SEQ ID NO: 166 |
| | | | protein | | | |
| 168 | EXOC7 | Q9UPT5 | Cytoskeletal | K714 | FGSVPFTKNPEkYIK | SEQ ID NO: 167 |
| | | | protein | | | |
| 169 | EXOC7 | Q9UPT5 | Cytoskeletal | K717 | FGSVPFTKNPEKYIk | SEQ ID NO: 168 |
| | | | protein | | | |
| 170 | K-ALPHA-1 | P68363 | Cytoskeletal | K352 | RSIQFVDWCPTGFk | SEQ ID NO: 169 |
| | | | protein | | | |
| 171 | KRT7 | P08729 | Cytoskeletal | K296 | FETLQAQAGkHGDDLR | SEQ ID NO: 170 |
| | | | protein | | | |
| 172 | KRT8 | P05787 | Cytoskeletal | K101 | TLNNkFASFIDK | SEQ ID NO: 171 |
| | | | protein | | | |
| 173 | KRT8 | P05787 | Cytoskeletal | K11 | VTQKSYkVSTSGPR | SEQ ID NO: 172 |
| | | | protein | | | |
| 174 | KRT8 | P05787 | Cytoskeletal | K121 | MPETkWSLLQQQK | SEQ ID NO: 173 |
| | | | protein | | | |
| 175 | KRT8 | P05787 | Cytoskeletal | K122 | MLETkWSLLQQQK | SEQ ID NO: 174 |
| | | | protein | | | |
| 176 | KRT8 | P05787 | Cytoskeletal | K285 | SRAEAESMYQIkYEELQSLAGK | SEQ ID NO: 175 |
| | | | protein | | | |
| 177 | KRT8 | P05787 | Cytoskeletal | K464 | AVVVkKIETR | SEQ ID NO: 176 |
| | | | protein | | | |
| 178 | KRT8 | P05787 | Cytoskeletal | K465 | AVVVKkIETR | SEQ ID NO: 177 |
| | | | protein | | | |
| 179 | KRT8 | P05787 | Cytoskeletal | K8 | VTQkSYKVSTSGPR | SEQ ID NO: 178 |
| | | | protein | | | |
| 180 | LMNA | P02545 | Cytoskeletal | K233 | LVEIDNGkQR | SEQ ID NO: 179 |
| | | | protein | | | |
| 181 | LMNA | P02545 | Cytoskeletal | K597 | TVLCGTCGQPADkASASGSGAQVGGPISS | SEQ ID NO: 180 |
| | | | protein | | GSSASSVTVTR | |
| 182 | LMNB1 | P20700 | Cytoskeletal | K271 | LYKEELEQTYHAkLENAR | SEQ ID NO: 181 |
| | | | protein | | | |
| 183 | MAP1B | P46821 | Cytoskeletal | K707 | ETPPkEVKKEVKKEEKKEVK | SEQ ID NO: 182 |
| | | | protein | | | |
| 184 | MAP1B | P46821 | Cytoskeletal | K714 | ETPPKEVKKEVkKEEKKEVK | SEQ ID NO: 183 |
| | | | protein | | | |
| 185 | MAP1B | P46821 | Cytoskeletal | K715 | ETPPKEVKKEVKkEEKKEVK | SEQ ID NO: 184 |
| | | | protein | | | |
| 186 | MAP1B | P46821 | Cytoskeletal | K719 | ETPPKEVKKEVKKEEKkEVK | SEQ ID NO: 185 |
| | | | protein | | | |
| 187 | MAP6 | Q6P3T0 | Cytoskeletal | K306 | kAKDKQAVSGQAAKK | SEQ ID NO: 186 |
| | | | protein | | | |
| 188 | MAP6 | Q6P3T0 | Cytoskeletal | K308 | KAkDKQAVSGQAAKK | SEQ ID NO: 187 |
| | | | protein | | | |
| 189 | MAPT | P10636 | Cytoskeletal | K311 | VQIVYkPVDLSK | SEQ ID NO: 188 |
| | | | protein | | | |
| 190 | MAPT | P10636 | Cytoskeletal | K369 | IGSLDNITHVPGGGNkK | SEQ ID NO: 189 |
| | | | protein | | | |
| 191 | MNS1 | Q8IYT6 | Cytoskeletal | K377 | kTMLAKFAEDDR | SEQ ID NO: 190 |
| | | | protein | | | |
| 192 | NEB | P20929 | Cytoskeletal | K345 | MNKKAGVAASkVKYK | SEQ ID NO: 191 |
| | | | protein | | | |
| 193 | NEB | P20929 | Cytoskeletal | K347 | MNKKAGVAASKVkYK | SEQ ID NO: 192 |
| | | | protein | | | |
| 194 | PLEC1 | Q15149 | Cytoskeletal | K3503 | TLLQGSGCLAGIYLEDTkEK | SEQ ID NO: 193 |
| | | | protein | | | |
| 195 | PLEC1 | Q15149 | Cytoskeletal | K953 | GRLPLLAVCDYk | SEQ ID NO: 194 |
| | | | protein | | | |
| 196 | PPHLN1 | Q8NEY8 | Cytoskeletal | K240 | WAAEkLEK | SEQ ID NO: 195 |
| | | | protein | | | |
| 197 | STMN1 | P16949 | Cytoskeletal | K119 | EAQMAAkLER | SEQ ID NO: 196 |
| | | | protein | | | |
| 198 | STMN1 | P16949 | Cytoskeletal | K53 | DLSLEEIQKkLEAAEER | SEQ ID NO: 197 |
| | | | protein | | | |
| 199 | STOML2 | Q9UJZ1 | Cytoskeletal | K233 | QAQILASEAEkAEQINQAAGEASAVLAK | SEQ ID NO: 198 |
| | | | protein | | | |
| 200 | TUBB2C | P68371 | Cytoskeletal | K103 | SGPFGQIFRPDNFVFGQSGAGNNWAk | SEQ ID NO: 199 |
| | | | protein | | | |
| 201 | TUBB2C | P68371 | Cytoskeletal | K297 | ALTVPELTQQMFDAk | SEQ ID NO: 200 |
| | | | protein | | | |
| 202 | VIM | P08670 | Cytoskeletal | K104 | TNEkVELQELNDR | SEQ ID NO: 201 |
| | | | protein | | | |
| 203 | VIM | P08670 | Cytoskeletal | K235 | KVESLQEEIAFLkK | SEQ ID NO: 202 |
| | | | protein | | | |
| 204 | XP_301899 | XP_301899 | Cytoskeletal | K61 | DSYVGNEAQSkR | SEQ ID NO: 203 |
| | | | protein | | | |
| 205 | K-ALPHA-1 | P68363 | Motor protein | K311 | HGkYMACCLLYR | SEQ ID NO: 204 |
| 206 | K-ALPHA-1 | P68363 | Cytoskeletal | K394 | LDHkFDLMYAKR | SEQ ID NO: 205 |
| | | | protein | | | |
| | | | Motor protein | | | |
| 207 | ARID4B | Q9NZB6 | DNA binding | K818 | TTGFYSGFSEVAEkR | SEQ ID NO: 206 |
| | | | protein | | | |
| 208 | CBX3 | Q13185 | DNA binding | K44 | RVVNGkVEYFLK | SEQ ID NO: 207 |
| | | | protein | | | |
| 209 | CHD4 | Q14839 | DNA binding | K1016 | GGGNQVSLLNVVMDLkK | SEQ ID NO: 208 |
| | | | protein | | | |
| 210 | HMG1L1 | Q9NQJ4 | DNA binding | K180 | GKPEAAKKGVVKAEk | SEQ ID NO: 209 |
| | | | protein | | | |
| 211 | HMGA1 | P17096 | DNA binding | K15 | SESSSKSSQPLASkQEK | SEQ ID NO: 210 |
| | | | protein | | | |
| 212 | HMGA1 | P17096 | DNA binding | K7 | SESSSkSSQPLASKQEK | SEQ ID NO: 211 |
| | | | protein | | | |
| 213 | HMGB2 | P26583 | DNA binding | K139 | LGEMWSEQSAkDK | SEQ ID NO: 212 |
| | | | protein | | | |
| 214 | HMGB3 | O15347 | DNA binding | K178 | kVEEEDEEQEEEEEEEEEEEDE | SEQ ID NO: 213 |
| | | | protein | | | |
| 215 | HMGN2 | P05204 | DNA binding | K13 | RKAEGDAKGGkAK | SEQ ID NO: 214 |
| | | | protein | | | |
| 216 | HMGN2 | P05204 | DNA binding | K76 | EGNNPAENGDAkTDQAQKAEGAGDAK | SEQ ID NO: 215 |
| | | | protein | | | |
| 217 | HMGN2 | P05204 | DNA binding | K90 | ADAGKEGNNPAENGDAKTDQAQKAEGAG | SEQ ID NO: 216 |
| | | | protein | | DAk | |
| 218 | HMGN4 | O00479 | DNA binding | K90 | DASTLQSQKAEGTGDAk | SEQ ID NO: 217 |
| | | | protein | | | |
| 219 | HNRPDL | O14979 | DNA binding | K180 | FGEVVDCTIkTDPVTGR | SEQ ID NO: 218 |
| | | | protein | | | |
| 220 | HNRPDL | O14979 | DNA binding | K302 | YHQIGSGkCEIK | SEQ ID NO: 219 |
| | | | protein | | | |
| 221 | LBR | Q14739 | DNA binding | K123 | LTPLILkPFGNSISR | SEQ ID NO: 220 |
| | | | protein | | | |
| 222 | LBR | Q14739 | DNA binding | K601 | YGVAWEkYCQR | SEQ ID NO: 221 |
| | | | protein | | | |
| 223 | MSH2 | P43246 | DNA binding | K73 | YMGPAGAkNLQSVVLSK | SEQ ID NO: 222 |
| | | | protein | | | |
| 224 | NEIL3 | Q8TAT5 | DNA binding | K244 | CRKAGLALSkHYKVYKR | SEQ ID NO: 223 |
| | | | protein | | | |
| 225 | NEIL3 | Q8TAT5 | DNA binding | K247 | CRKAGLALSKHYkVYKR | SEQ ID NO: 224 |
| | | | protein | | | |
| 226 | NEIL3 | Q8TAT5 | DNA binding | K250 | CRKAGLALSKHYKVYkR | SEQ ID NO: 225 |
| | | | protein | | | |
| 227 | POLR2L | P62875 | DNA binding | K41 | IVGNKWEAYLGLLQAEYTEGDALDALGLkR | SEQ ID NO: 226 |
| | | | protein | | | |
| 228 | PURB | Q96QR8 | DNA binding | K267 | AWGkFGGAFCR | SEQ ID NO: 227 |
| | | | protein | | | |
| 229 | RAD51L3 | O75771 | DNA binding | K261 | DRDSGRLkPALGR | SEQ ID NO: 228 |
| | | | protein | | | |
| 230 | RAG1 | P15918 | DNA binding | K983 | KMNARQSkCYEMEDVLKHHWLYTSKYLQK | SEQ ID NO: 229 |
| | | | protein | | | |
| 231 | RPA1 | P27694 | DNA binding | K163 | AYGASkTFGKAAGPSLSHTSGGTQSK | SEQ ID NO: 230 |
| | | | protein | | | |
| 232 | RPA1 | P27694 | DNA binding | K167 | AYGASKTFGkAAGPSLSHTSGGTQSK | SEQ ID NO: 231 |
| | | | protein | | | |
| 233 | RPA1 | P27694 | DNA binding | K196 | VVPIASLTPYQSkWTICAR | SEQ ID NO: 232 |
| | | | protein | | | |
| 234 | SAFB | Q15424 | DNA binding | K83 | AIEDEGGNPDEIEITSEGNkK | SEQ ID NO: 233 |
| | | | protein | | | |
| 235 | SAFB | Q15424 | DNA binding | K84 | AIEDEGGNPDEIEITSEGNKk | SEQ ID NO: 234 |
| | | | protein | | | |
| 236 | SMARCC1 | Q92922 | DNA binding | K359 | SQkEEDEQEDLTKDMEDPTPVPNIEEVVLPK | SEQ ID NO: 235 |
| | | | protein | | | |
| 237 | SON | P18583 | DNA binding | K16 | SFVVSkFR | SEQ ID NO: 236 |
| | | | protein | | | |
| 238 | SON | P18583 | DNA binding | K2055 | LTDLDkAQLLEIAK | SEQ ID NO: 237 |
| | | | protein | | | |
| 239 | SON | P18583 | DNA binding | K2063 | RLTDLDKAQLLEIAk | SEQ ID NO: 238 |
| | | | protein | | | |
| 240 | SYCP1 | Q15431 | DNA binding | K111 | LYkEAEK | SEQ ID NO: 239 |
| | | | protein | | | |
| 241 | TMPO | P42167 | DNA binding | K334 | AEVGEkTEER | SEQ ID NO: 240 |
| | | | protein | | | |
| 242 | TREX2 | Q99871 | DNA binding | K88 | VWPSLQDRFSSLkGVPTEVK | SEQ ID NO: 241 |
| | | | protein | | | |
| 243 | TREX2 | Q99871 | DNA binding | K95 | VWPSLQDRFSSLKGVPTEVk | SEQ ID NO: 242 |
| | | | protein | | | |
| 244 | TSNAX | Q99598 | DNA binding | K252 | QSLAkVENACYALK | SEQ ID NO: 243 |
| | | | protein | | | |
| 245 | WBP11 | Q9Y2W2 | DNA binding | K13 | SGkFMNPTDQAR | SEQ ID NO: 244 |
| | | | protein | | | |
| 246 | WDHD1 | O75717 | DNA binding | K1127 | LSAFAFkQE | SEQ ID NO: 245 |
| | | | protein | | | |
| 247 | XRCC6 | P12956 | DNA binding | K510 | NLEALALDLMEPEQAVDLTLPkVEAMNKR | SEQ ID NO: 246 |
| | | | protein | | | |
| 248 | ZCCHC3 | Q9NUD5 | DNA binding | K124 | RkKAEAAAAAMATPAR | SEQ ID NO: 247 |
| | | | protein | | | |
| 249 | ZCCHC3 | Q9NUD5 | DNA binding | K125 | RKkAEAAAAAMATPAR | SEQ ID NO: 248 |
| | | | protein | | | |
| 250 | ZMYM3 | Q14202 | DNA binding | K489 | FCNTTCLGAYkK | SEQ ID NO: 249 |
| | | | protein | | | |
| 251 | ZNF146 | Q15072 | DNA binding | K135 | ECGkTFSGKSNLTEHEK | SEQ ID NO: 250 |
| | | | protein | | | |
| 252 | ZNF146 | Q15072 | DNA binding | K140 | ECGKTFSGkSNLTEHEK | SEQ ID NO: 251 |
| | | | protein | | | |
| 253 | ZNF22 | P17026 | DNA binding | K18 | SSSQGkAYENKR | SEQ ID NO: 252 |
| | | | protein | | | |
| 254 | NCL | P19338 | DNA binding | K116 | GATPGkALVATPGKK | SEQ ID NO: 253 |
| | | | protein | | | |
| | | | Helicase | | | |
| | | | RNA binding | | | |
| | | | protein | | | |
| 255 | NCL | P19338 | DNA binding | K124 | GATPGKALVATPGkK | SEQ ID NO: 254 |
| | | | protein | | | |
| | | | Helicase | | | |
| | | | RNA binding | | | |
| | | | protein | | | |
| 256 | NCL | P19338 | DNA binding | K125 | GATPGKALVATPGKk | SEQ ID NO: 255 |
| | | | protein | | | |
| | | | Helicase | | | |
| | | | RNA binding | | | |
| | | | protein | | | |
| 257 | NCL | P19338 | DNA binding | K132 | GAAIPAkGAK | SEQ ID NO: 256 |
| | | | protein | | | |
| | | | Helicase | | | |
| | | | RNA binding | | | |
| | | | protein | | | |
| 258 | NCL | P19338 | DNA binding | K403 | NLPYkVTQDELK | SEQ ID NO: 257 |
| | | | protein | | | |
| | | | Helicase | | | |
| | | | RNA binding | | | |
| | | | protein | | | |
| 259 | NCL | P19338 | DNA binding | K545 | EALNSCNkR | SEQ ID NO: 258 |
| | | | protein | | | |
| | | | Helicase | | | |
| | | | RNA binding | | | |
| | | | protein | | | |
| 260 | HNRPU | Q00839 | DNA binding | K21 | VSELKEELkK | SEQ ID NO: 259 |
| | | | protein | | | |
| | | | RNA binding | | | |
| | | | protein | | | |
| 261 | HNRPU | Q00839 | DNA binding | K351 | HLYTkDIDIHEVR | SEQ ID NO: 260 |
| | | | protein | | | |
| | | | RNA binding | | | |
| | | | protein | | | |
| 262 | HNRPU | Q00839 | DNA binding | K564 | APQCLGkFIEIAAR | SEQ ID NO: 261 |
| | | | protein | | | |
| | | | RNA binding | | | |
| | | | protein | | | |
| 263 | HNRPU | Q00839 | DNA binding | K813 | NQSQGYNQWQQGQFWGQkPWSQHYHQGYY | SEQ ID NO: 262 |
| | | | protein | | | |
| | | | RNA binding | | | |
| | | | protein | | | |
| 264 | SAFB | Q15424 | DNA binding | K293 | ADSLLAVVkREPAEQPGDGER | SEQ ID NO: 263 |
| | | | protein | | | |
| | | | Unknown | | | |
| | | | function | | | |
| 265 | APEX1 | P27695 | DNA repair | K85 | GLDWVkEEAPDILCLQETK | SEQ ID NO: 264 |
| 266 | PARP1 | P09874 | DNA repair | K105 | GQDGIGSkAEK | SEQ ID NO: 265 |
| 267 | PARP1 | P09874 | DNA repair | K621 | LYEEkTGNAWHSK | SEQ ID NO: 266 |
| 268 | RAD18 | Q9NS91 | DNA repair | K370 | IAGMSQkTVTITK | SEQ ID NO: 267 |
| 269 | RAD50 | Q92878 | DNA repair | K959 | NIHGYMkDIENYIQDGKDDYKK | SEQ ID NO: 268 |
| 270 | ATRX | P46100 | DNA repair | K1933 | KKKkGKKGKK | SEQ ID NO: 269 |
| | | | Helicase | | | |
| 271 | ATRX | P46100 | DNA repair | K1935 | KKKKGkKGKK | SEQ ID NO: 270 |
| | | | Helicase | | | |
| 272 | ATRX | P46100 | DNA repair | K1936 | KKKKGKkGKK | SEQ ID NO: 271 |
| | | | Helicase | | | |
| 273 | ATRX | P46100 | DNA repair | K1939 | KKKKGKKGKk | SEQ ID NO: 272 |
| | | | Helicase | | | |
| 274 | ATRX | P46100 | DNA repair | K967 | KVQDGLSDIAEkFLK | SEQ ID NO: 273 |
| | | | Helicase | | | |
| 275 | BAZ1B | Q9UIG0 | DNA replication | K409 | GRSkGILNGQK | SEQ ID NO: 274 |
| 276 | BAZ1B | Q9UIG0 | DNA replication | K416 | GRSKGILNGQk | SEQ ID NO: 275 |
| 277 | CHAF1A | Q13111 | DNA replication | K449 | TLAGSCGkFAPFEIK | SEQ ID NO: 276 |
| 278 | NAP1L1 | P55209 | DNA replication | K263 | MRSEPDDSDPFSFDGPEIMGCTGCQIDWkK | SEQ ID NO: 277 |
| 279 | NAP1L1 | P55209 | DNA replication | K264 | MRSEPDDSDPFSFDGPEIMGCTGCQIDWKk | SEQ ID NO: 278 |
| 280 | POLD3 | Q15054 | DNA replication | K286 | SSkKAEPVKVLQKEKKRGK | SEQ ID NO: 279 |
| 281 | POLD3 | Q15054 | DNA replication | K287 | SSKkAEPVKVLQKEKKRGK | SEQ ID NO: 280 |
| 282 | POLD3 | Q15054 | DNA replication | K298 | SSKKAEPVKVLQKEkKRGK | SEQ ID NO: 281 |
| 283 | POLD3 | Q15054 | DNA replication | K302 | SSKKAEPVKVLQKEKKRGk | SEQ ID NO: 282 |
| 284 | CHERP | Q8IWX8 | Endoplasmic | K239 | QARELLAALQk | SEQ ID NO: 283 |
| | | | reticulum | | | |
| 285 | DNAJB11 | Q9UBS4 | Endoplasmic | K344 | EGIKQLLkQGSVQK | SEQ ID NO: 284 |
| | | | reticulum | | | |
| 286 | JPH2 | Q9BR39 | Endoplasmic | K647 | GLTkAGAK | SEQ ID NO: 285 |
| | | | reticulum | | | |
| 287 | JPH2 | Q9BR39 | Endoplasmic | K651 | GLTKAGAkK | SEQ ID NO: 286 |
| | | | reticulum | | | |
| 288 | SEC63 | Q9UGP8 | Endoplasmic | K527 | SkKKKPLK | SEQ ID NO: 287 |
| | | | reticulum | | | |
| 289 | VAPA | Q9P0L0 | Endoplasmic | K17 | HEQILVLDPPTDLk | SEQ ID NO: 288 |
| | | | reticulum | | | |
| 290 | AHCY | P23526 | Enzyme, | K188 | SkFDNLYGCR | SEQ ID NO: 289 |
| | | | cellular | | | |
| | | | metabolism | | | |
| 291 | ALDOA | P04075 | Enzyme, | K108 | GGVVGIkVDK | SEQ ID NO: 290 |
| | | | cellular | | | |
| | | | metabolism | | | |
| 292 | ALDOA | P04075 | Enzyme, | K200 | YASICQQNGIVPIVEPEILPDGDHDLkR | SEQ ID NO: 291 |
| | | | cellular | | | |
| | | | metabolism | | | |
| 293 | ALDOA | P04075 | Enzyme, | K230 | ALSDHHIYLEGTLLkPNMVTPGHACTQK | SEQ ID NO: 292 |
| | | | cellular | | | |
| | | | metabolism | | | |
| 294 | ALDOA | P04075 | Enzyme, | K42 | GILAADESTGSIAkR | SEQ ID NO: 293 |
| | | | cellular | | | |
| | | | metabolism | | | |
| 295 | ATP5A1 | P25705 | Enzyme, | K498 | GYLDkLEPSK | SEQ ID NO: 294 |
| | | | cellular | | | |
| | | | metabolism | | | |
| 296 | ATP5A1 | P25705 | Enzyme, | K539 | ISEQSDAkLK | SEQ ID NO: 295 |
| | | | cellular | | | |
| | | | metabolism | | | |
| 297 | DUT | P33316 | Enzyme, | K251 | GSGGFGSTGkN | SEQ ID NO: 296 |
| | | | cellular | | | |
| | | | metabolism | | | |
| 298 | ENO1 | P06733 | Enzyme, | K193 | IGAEVYHNLkNVIK | SEQ ID NO: 297 |
| | | | cellular | | | |
| | | | metabolism | | | |
| 299 | ENO1 | P06733 | Enzyme, | K199 | EkYGKDATNVGDEGGFAPNILENK | SEQ ID NO: 298 |
| | | | cellular | | | |
| | | | metabolism | | | |
| 300 | ENO1 | P06733 | Enzyme, | K256 | SGkYDLDFKSPDDPSR | SEQ ID NO: 299 |
| | | | cellular | | | |
| | | | metabolism | | | |
| 301 | ENO1 | P06733 | Enzyme, | K281 | YISPDQLADLYk | SEQ ID NO: 300 |
| | | | cellular | | | |
| | | | metabolism | | | |
| 302 | ENO1 | P06733 | Enzyme, | K306 | SFIKDYPVVSIEDPFDQDDWGAWQk | SEQ ID NO: 301 |
| | | | cellular | | | |
| | | | metabolism | | | |
| 303 | ENO1 | P06733 | Enzyme, | K335 | AVNEkSCNCLLLK | SEQ ID NO: 302 |
| | | | cellular | | | |
| | | | metabolism | | | |
| 304 | ENO1 | P06733 | Enzyme, | K343 | SCNCLLLkVNQIGSVTESLQACK | SEQ ID NO: 303 |
| | | | cellular | | | |
| | | | metabolism | | | |
| 305 | GAPDH | P04406 | Enzyme, | K194 | TVDGPSGkLWR | SEQ ID NO: 304 |
| | | | cellular | | | |
| | | | metabolism | | | |
| 306 | GAPDH | P04406 | Enzyme, | K227 | VIPELNGkLTGMAFR | SEQ ID NO: 305 |
| | | | cellular | | | |
| | | | metabolism | | | |
| 307 | GAPDH | P04406 | Enzyme, | K259 | LEKPAKYDDIkK | SEQ ID NO: 306 |
| | | | cellular | | | |
| | | | metabolism | | | |
| 308 | GAPDH | P04406 | Enzyme, | K61 | FHGTVkAENGK | SEQ ID NO: 307 |
| | | | cellular | | | |
| | | | metabolism | | | |
| 309 | GNPDA1 | P46926 | Enzyme, | K51 | YFTLGLPTGSTPLGCYKk | SEQ ID NO: 308 |
| | | | cellular | | | |
| | | | metabolism | | | |
| 310 | LDHA | P00338 | Enzyme, | K126 | FIIPNVVkYSPNCK | SEQ ID NO: 309 |
| | | | cellular | | | |
| | | | metabolism | | | |
| 311 | LDHA | P00338 | Enzyme, | K14 | DQLIYNLLkEEQTPQNK | SEQ ID NO: 310 |
| | | | cellular | | | |
| | | | metabolism | | | |
| 312 | LDHA | P00338 | Enzyme, | K22 | DQLIYNLLKEEQTPQNk | SEQ ID NO: 311 |
| | | | cellular | | | |
| | | | metabolism | | | |
| 313 | LDHA | P00338 | Enzyme, | K5 | ATLkDQLIYNLLK | SEQ ID NO: 312 |
| | | | cellular | | | |
| | | | metabolism | | | |
| 314 | LDHB | P07195 | Enzyme, | K332 | SADTLWDIQKDLkDL | SEQ ID NO: 313 |
| | | | cellular | | | |
| | | | metabolism | | | |
| 315 | PGAM1 | P18669 | Enzyme, | K100 | HYGGLTGLNkAETAAK | SEQ ID NO: 314 |
| | | | cellular | | | |
| | | | metabolism | | | |
| 316 | PGK1 | P00558 | Enzyme, | K75 | SVVLMSHLGRPDGVPMPDkYSLEPVAVELK | SEQ ID NO: 315 |
| | | | cellular | | | |
| | | | metabolism | | | |
| 317 | PKLR | P30613 | Enzyme, | K305 | GDLGIEIPAEkVFLAQK | SEQ ID NO: 316 |
| | | | cellular | | | |
| | | | metabolism | | | |
| 318 | PKM2 | P14786 | Enzyme, | K162 | CDENILWLDYk | SEQ ID NO: 317 |
| | | | cellular | | | |
| | | | metabolism | | | |
| 319 | PKM2 | P14786 | Enzyme, | K206 | GADFLVTEVENGGSLGSkK | SEQ ID NO: 318 |
| | | | cellular | | | |
| | | | metabolism | | | |
| 320 | PKM2 | P14786 | Enzyme, | K230 | GVNLPGAAVDLPAVSEKDIQDLk | SEQ ID NO: 319 |
| | | | cellular | | | |
| | | | metabolism | | | |
| 321 | PKM2 | P14786 | Enzyme, | K135 | GSGTAEVELkK | SEQ ID NO: 320 |
| | | | cellular | | | |
| | | | metabolism | | | |
| | | | Unknown | | | |
| | | | function | | | |
| 322 | FASN | P49327 | Enzyme, misc. | K1116 | RQQEQQVPILEkFCFTPHTEEGCLSER | SEQ ID NO: 321 |
| 323 | FASN | P49327 | Enzyme, misc. | K1771 | FLEIGkFDLSQNHPLGMAIFLK | SEQ ID NO: 322 |
| 324 | FASN | P49327 | Enzyme, misc. | K1995 | DGLLENQTPEFFQDVCKPkYSGTLNLDR | SEQ ID NO: 323 |
| 325 | FASN | P49327 | Enzyme, misc. | K213 | LGMLSPEGTCkAFDTAGNGYCR | SEQ ID NO: 324 |
| 326 | FASN | P49327 | Enzyme, misc. | K2471 | TGGAYGEDLGADYNLSQVCDGk | SEQ ID NO: 325 |
| 327 | FASN | P49327 | Enzyme, misc. | K298 | SLYQSAGVAPESFEYIEAHGTGTkVGDPQE | SEQ ID NO: 326 |
| | | | | | LNGITR | |
| 328 | FASN | P49327 | Enzyme, misc. | K436 | TPEAVQkLLEQGLR | SEQ ID NO: 327 |
| 329 | FASN | P49327 | Enzyme, misc. | K673 | EGVFAkEVR | SEQ ID NO: 328 |
| 330 | FASN | P49327 | Enzyme, misc. | K70 | FDASFFGVHPkQAHTMDPQLR | SEQ ID NO: 329 |
| 331 | FASN | P49327 | Enzyme, misc. | K786 | GLKPSCTIIPLMkK | SEQ ID NO: 330 |
| 332 | GLB1 | P16278 | Enzyme, misc. | K493 | VNYGAYINDFk | SEQ ID NO: 331 |
| 333 | HIBCH | Q6NVY1 | Enzyme, misc. | K353 | AVLIDkDQSPK | SEQ ID NO: 332 |
| 334 | MTHFD1 | P11586 | Enzyme, misc. | K819 | AAQAPSSFQLLYDLk | SEQ ID NO: 333 |
| 335 | PECI | O75521 | Enzyme, misc. | K324 | EREkLHAVNAEECNVLQGR | SEQ ID NO: 334 |
| 336 | PECI | O75521 | Enzyme, misc. | K6 | ASQkDFENSMNQVK | SEQ ID NO: 335 |
| 337 | COL22A1 | Q8NFW1 | Extracellular | K1401 | GDPGIkGDKGPPGGK | SEQ ID NO: 336 |
| | | | matrix | | | |
| 338 | COL22A1 | Q8NFW1 | Extracellular | K1404 | GDPGIKGDkGPPGGK | SEQ ID NO: 337 |
| | | | matrix | | | |
| 339 | COL22A1 | Q8NFW1 | Extracellular | K1410 | GDPGIKGDKGPPGGk | SEQ ID NO: 338 |
| | | | matrix | | | |
| 340 | COL5A3 | P25940 | Extracellular | K247 | kGKGKGRKK | SEQ ID NO: 339 |
| | | | matrix | | | |
| 341 | COL5A3 | P25940 | Extracellular | K249 | KGkGKGRKK | SEQ ID NO: 340 |
| | | | matrix | | | |
| 342 | COL5A3 | P25940 | Extracellular | K251 | KGKGkGRKK | SEQ ID NO: 341 |
| | | | matrix | | | |
| 343 | COL5A3 | P25940 | Extracellular | K255 | KGKGKGRKk | SEQ ID NO: 342 |
| | | | matrix | | | |
| 344 | COL8A1 | P27658 | Extracellular | K106 | MGKEAVPkKGKEIPLASLR | SEQ ID NO: 343 |
| | | | matrix | | | |
| 345 | LAD1 | O00515 | Extracellular | K259 | LVSEkASIFEK | SEQ ID NO: 344 |
| | | | matrix | | | |
| 346 | ARHGDIA | P52565 | G protein | K141 | IDkTDYMVGSYGPR | SEQ ID NO: 345 |
| | | | regulator, | | | |
| | | | misc. | | | |
| 347 | IPO8 | O15397 | G protein | K380 | MkFDIFEDYASPTTAAQTLLYTAAK | SEQ ID NO: 346 |
| | | | regulator, | | | |
| | | | misc. | | | |
| 348 | IQGAP2 | Q13576 | G protein | K1467 | LDGkGEPKGAKR | SEQ ID NO: 347 |
| | | | regulator, | | | |
| | | | misc. | | | |
| 349 | IQGAP2 | Q13576 | G protein | K1471 | LDGKGEPkGAKR | SEQ ID NO: 348 |
| | | | regulator, | | | |
| | | | misc. | | | |
| 350 | IQGAP2 | Q13576 | G protein | K1474 | LDGKGEPKGAkR | SEQ ID NO: 349 |
| | | | regulator, | | | |
| | | | misc. | | | |
| 351 | RANBP1 | P43487 | G protein | K150 | FLNAENAQkFK | SEQ ID NO: 350 |
| | | | regulator, | | | |
| | | | misc. | | | |
| 352 | RANBP1 | P43487 | G protein | K183 | VAEkLEALSVKEETKEDAEEKQ | SEQ ID NO: 351 |
| | | | regulator, | | | |
| | | | misc. | | | |
| 353 | RANBP1 | P43487 | G protein | K190 | VAEKLEALSVkEETKEDAEEKQ | SEQ ID NO: 352 |
| | | | regulator, | | | |
| | | | misc. | | | |
| 354 | RAN | P62826 | G protein, | K127 | VCENIPIVLCGNKVDIkDR | SEQ ID NO: 353 |
| | | | monomeric | | | |
| | | | (non-Rab) | | | |
| 355 | RAN | P62826 | G protein, | K71 | FNVWDTAGQEkFGGLR | SEQ ID NO: 354 |
| | | | monomeric | | | |
| | | | (non-Rab) | | | |
| 356 | RANGAP1 | P46060 | GTPase activating | K26 | TQVAGGQLSFkGK | SEQ ID NO: 355 |
| | | | protein, misc. | | | |
| 357 | RANGAP1 | P46060 | GTPase activating | K524 | LLVHMGLLkSEDKVK | SEQ ID NO: 356 |
| | | | protein, misc. | | | |
| 358 | ARHGAP26 | Q9UNA1 | GTPase activating | K128 | EQIGAAkEAKKK | SEQ ID NO: 357 |
| | | | protein, Rac/Rho | | | |
| 359 | RACGAP1 | Q9P2W2 | GTPase activating | K632 | QGNFFASPMLk | SEQ ID NO: 358 |
| | | | protein, Rac/Rho | | | |
| 360 | RALB | P11234 | GTPase activating | K179 | EIRTkKMSENKDKNGK | SEQ ID NO: 359 |
| | | | protein, Ras | | | |
| 361 | RALB | P11234 | GTPase activating | K190 | EIRTKKMSENKDKNGk | SEQ ID NO: 360 |
| | | | protein, Ras | | | |
| 362 | RASA2 | Q15283 | GTPase activating | K124 | DLRIGKVAIkK | SEQ ID NO: 361 |
| | | | protein, Ras | | | |
| 363 | RGS10 | O43665 | GTPase activating | K45 | WAASLENLLEDPEGVkR | SEQ ID NO: 362 |
| | | | protein, RGS | | | |
| 364 | RCC2 | Q9P258 | Guanine | K293 | GNLYSFGSPEYGQLGHNSDGkFIAR | SEQ ID NO: 363 |
| | | | nucleotide | | | |
| | | | exchange | | | |
| | | | factor, misc. | | | |
| 365 | ARHGEF11 | O15085 | Guanine | K925 | DQCREILKYVNEAVk | SEQ ID NO: 364 |
| | | | nucleotide | | | |
| | | | exhcange | | | |
| | | | factor, | | | |
| | | | Rac/Rho | | | |
| 366 | DDX18 | Q9NVP1 | Helicase | K126 | KMVNDAEPDTkKAK | SEQ ID NO: 365 |
| 367 | DDX18 | Q9NVP1 | Helicase | K458 | YHYELLNYIDLPVLAIHGkQK | SEQ ID NO: 366 |
| 368 | DDX21 | Q9NR30 | Helicase | K18 | SDAGLESDTAMkK | SEQ ID NO: 367 |
| 369 | DDX24 | Q9GZR7 | Helicase | K17 | QSSCGkFQTK | SEQ ID NO: 368 |
| 370 | DDX42 | Q86XP3 | Helicase | K686 | GNNNVMSNYEAYkPSTGAMGDR | SEQ ID NO: 369 |
| 371 | DHX15 | O43143 | Helicase | K17 | HRLDLGEDYPSGkK | SEQ ID NO: 370 |
| 372 | DHX36 | Q9H2U1 | Helicase | K845 | VAkIRLNLGKKR | SEQ ID NO: 371 |
| 373 | DHX36 | Q9H2U1 | Helicase | K853 | VAKIRLNLGKkR | SEQ ID NO: 372 |
| 374 | DHX9 | Q08211 | Helicase | K1037 | SSVNCPFSSQDMk | SEQ ID NO: 373 |
| 375 | DHX9 | Q08211 | Helicase | K14 | NFLYAWCGkR | SEQ ID NO: 374 |
| 378 | RUVBL1 | Q9Y265 | Helicase | K456 | ILADQQDKYMk | SEQ ID NO: 375 |
| 377 | RUVBL2 | Q9Y230 | Helicase | K417 | kGTEVQVDDIK | SEQ ID NO: 376 |
| 378 | XRCC5 | P13010 | Helicase | K265 | IAAYkSILQER | SEQ ID NO: 377 |
| 379 | XRCC5 | P13010 | Helicase | K565 | KKDQVTAQEIFQDNHEDGPTAkK | SEQ ID NO: 378 |
| 380 | DDX3X | O00571 | Helicase | K118 | SGFGkFER | SEQ ID NO: 379 |
| | | | binding | | | |
| | | | protein | | | |
| 381 | CNP | P09543 | Hydrolase, | K175 | NQWQLSADDLkK | SEQ ID NO: 380 |
| | | | esterase | | | |
| 382 | DFFA | O00273 | Hydrolase, | K324 | ASPPGDLQNPkR | SEQ ID NO: 381 |
| | | | esterase | | | |
| 383 | EXO1 | Q9UNW0 | Hydrolase, | K482 | NkFATFLQR | SEQ ID NO: 382 |
| | | | esterase | | | |
| 384 | THEX1 | Q81V48 | Hydrolase, | K99 | LETRGVkDVLK | SEQ ID NO: 383 |
| | | | esterase | | | |
| 385 | AGMAT | Q9BSE5 | Hydrolase, | K217 | CVDEGLLDCkR | SEQ ID NO: 384 |
| | | | non-esterase | | | |
| 386 | PADI1 | Q9ULC6 | Hydrolase, | K226 | GGNSLSDYk | SEQ ID NO: 385 |
| | | | non-esterase | | | |
| 387 | A2M | P01023 | Inhibitor protein | K1162 | ALLAYAFALAGNQDk | SEQ ID NO: 386 |
| 388 | PAK1IP1 | Q96T87 | Inhibitor protein | K817 | ATKESGLISTKkRKMVEMLEKK | SEQ ID NO: 387 |
| 389 | PAK1IP1 | Q96T87 | Inhibitor protein | K826 | ATKESGLISTKKRKMVEMLEkK | SEQ ID NO: 388 |
| 390 | PEBP1 | P30086 | Inhibitor protein | K132 | YVWNLVYEQDRPLkCDEPILSNR | SEQ ID NO: 389 |
| 391 | PEBP1 | P30086 | Inhibitor protein | K80 | LYTLVLTDPDAPSRKDPkYR | SEQ ID NO: 390 |
| 392 | SUMO2 | P61956 | Inhibitor protein | K11 | EGVkTENNDHINLK | SEQ ID NO: 391 |
| 393 | PIN1 | Q13526 | Isomerase | K46 | VYYFNHITNASQWERPSGNSSSGGkNGQGEPAR | SEQ ID NO: 392 |
| 394 | PPI1 | P62937 | Isomerase | K118 | HTGPGILSMANAGPNTNGSQFFICTAkTEWLDGK | SEQ ID NO: 393 |
| 395 | PPIA | P62937 | Isomerase | K131 | TEWLDGKHVVFGkVK | SEQ ID NO: 394 |
| 396 | PPIA | P62937 | Isomerase | K49 | GFGYkGSCFHR | SEQ ID NO: 395 |
| 397 | PPIA | P62937 | Isomerase | K76 | HNGTGGkSIYGEKFEDENFILK | SEQ ID NO: 396 |
| 398 | PPIA | P62937 | Isomerase | K82 | SIYGEkFEDENFILK | SEQ ID NO: 397 |
| 399 | TOP1 | P11387 | Isomerase | K172 | KLEEEEDGkLK | SEQ ID NO: 398 |
| 400 | TOP2A | P11388 | Isomerase | K1276 | kQTTLAFKPIKKGKKR | SEQ ID NO: 399 |
| 401 | TOP2A | P11388 | Isomerase | K1287 | KQTTLAFKPIKkGKKR | SEQ ID NO: 400 |
| 402 | TOP2A | P11388 | Isomerase | K1289 | KQTTLAFKPIKKGkKR | SEQ ID NO: 401 |
| 403 | TPI1 | P60174 | Isomerase | K248 | ELASQPDVDGFLVGGASLKPEFVDIINAkQ | SEQ ID NO: 402 |
| 404 | TPI1 | P60174 | Isomerase | K69 | IAVAAQNCYk | SEQ ID NO: 403 |
| 405 | AK3L1 | P27144 | Kinase (non- | K179 | DVAkPVIELYK | SEQ ID NO: 404 |
| | | | protein) | | | |
| 406 | AK3L1 | P27144 | Kinase (non- | K186 | DVAKPVIELYkSR | SEQ ID NO: 405 |
| | | | protein) | | | |
| 407 | ALDH18A1 | P54886 | Kinase (non- | K649 | IHAGPkFASYLTFSPSEVK | SEQ ID NO: 406 |
| | | | protein) | | | |
| 408 | NME2 | P22392 | Kinase (non- | K143 | EISLWFKPEELVDYk | SEQ ID NO: 407 |
| | | | protein) | | | |
| 409 | PRKDC | P78527 | Kinase, lipid | K3691 | ECSPWMSDFkVEFLR | SEQ ID NO: 408 |
| | | | Protein kinase, | | | |
| | | | Ser/Thr (non- | | | |
| | | | receptor) | | | |
| 410 | NRG1 | Q02297 | Ligand, receptor | K10 | KEGRGkGKGKKK | SEQ ID NO: 409 |
| | | | tyrosine kinase | | | |
| 411 | NRG1 | Q02297 | Ligand, receptor | K15 | KEGRGKGKGKkK | SEQ ID NO: 410 |
| | | | tyrosine kinase | | | |
| 412 | NRG1 | Q02297 | Ligand, receptor | K16 | KEGRGKGKGKKk | SEQ ID NO: 411 |
| | | | tyrosine kinase | | | |
| 413 | ACSS1 | Q9NUB1 | Ligase | K396 | LLLkYGDAWVK | SEQ ID NO: 412 |
| 414 | ACSS2 | Q9NR19 | Ligase | K418 | LLMkFGDEPVTK | SEQ ID NO: 413 |
| 415 | EPRS | P07814 | Ligase | K228 | AYVDDTPAEQMkAER | SEQ ID NO: 414 |
| 416 | EPRS | P07814 | Ligase | K425 | SvvNMEWDKIWAFNkK | SEQ ID NO: 415 |
| 417 | EPRS | P07814 | Ligase | K426 | SvvNMEWDKIWAFNKk | SEQ ID NO: 416 |
| 418 | IARS2 | Q9NSE4 | Ligase | K222 | SYkPVFWSPSSR | SEQ ID NO: 417 |
| 419 | PAICS | P22234 | Ligase | K110 | IATGSFLkR | SEQ ID NO: 418 |
| 420 | FABP5 | Q01469 | Lipid | K55 | NLTIkTESTLK | SEQ ID NO: 419 |
| | | | binding | | | |
| | | | protein | | | |
| 421 | FABP5 | Q01469 | Lipid | K71 | TTQFSGTLGEkFEENTADGR | SEQ ID NO: 420 |
| | | | binding | | | |
| | | | protein | | | |
| 422 | FABP5 | Q01469 | Lipid | K72 | TTQFSCTLGEkFEETTADGR | SEQ ID NO: 421 |
| | | | binding | | | |
| | | | protein | | | |
| 423 | PLEK | P08567 | Lipid | K64 | GSTLTSPCQDFGkR | SEQ ID NO: 422 |
| | | | binding | | | |
| | | | protein | | | |
| 424 | SCP2 | P22307 | Lipid | K453 | KLEEEGEQFVkK | SEQ ID NO: 423 |
| | | | binding | | | |
| | | | protein | | | |
| 425 | ADSL | P30566 | Lyase | K295 | QQIGSSAMPYkR | SEQ ID NO: 424 |
| 426 | EHHADH | Q08426 | Lyase | K219 | LCNKPIQSLPNMDSIFSEALLkMR | SEQ ID NO: 425 |
| 427 | EHHADH | Q08426 | Lyase | K346 | MITSVLEkEASK | SEQ ID NO: 426 |
| 428 | EHHADH | Q08426 | Lyase | K584 | GWYQYDkPLGR | SEQ ID NO: 427 |
| 429 | FH | P07954 | Lyase | K256 | THTQDAVPLTLGQEFSGYVQQVkYAMTR | SEQ ID NO: 428 |
| 430 | HADHA | P40939 | Lyase | K326 | TGIEQGSDAGYLCESQkFGELVMTK | SEQ ID NO: 429 |
| 431 | HADHA | P40939 | Lyase | K406 | GQQQVFkGLNDK | SEQ ID NO: 430 |
| 432 | HADHA | P40939 | Lyase | K460 | VLkEVEAVIPDHCIFASNTSALPISEIAAVSK | SEQ ID NO: 431 |
| 433 | HADHA | P40939 | Lyase | K644 | GFYIYQEGVkR | SEQ ID NO: 432 |

The short name for each protein in which acetylation site has presently been identified is provided in Column A, and its SwissProt accession number (human) is provided Column B. The protein type/group into which each protein falls is provided in Column C. The identified lysine residue at which acetylation occurs in a given protein is identified in Column D, and the amino acid sequence of the acetylation site encompassing the lysine residue is provided in Column E (lower case k=the lysine (identified in Column D)) at which acetylation occurs. Table 1 above is identical to FIG. 2, except that the latter includes the disease and cell type(s) in which the particular acetylation site was identified (Columns F and G).

The identification of these 432 acetylation sites is described in more detail in Part A below and in Example 1.

### DEFINITIONS

As used herein, the following terms have the meanings indicated:

“Antibody” or “antibodies” refers to all types of immunoglobulins, including IgG, IgM, IgA, IgD, and IgE, including F_{ab} or antigen-recognition fragments thereof, including chimeric, polyclonal, and monoclonal antibodies. The term “does not bind” with respect to an antibody's binding to one acetyl-form of a sequence means does not substantially react with as compared to the antibody's binding to the other acetyl-form of the sequence for which the antibody is specific.

“Protein acetylation signaling protein” means any protein (or polypeptide derived therefrom) enumerated in Column A of Table 1/FIG. 2, which is disclosed herein as being acetylated in one or more of the disclosed cell line(s). Protein acetylation signaling proteins may include, but are not limited to histone deacetylases (HDACs) and histone acetyltransferases (HATs).

“Heavy-isotope labeled peptide” (used interchangeably with AQUA peptide) means a peptide comprising at least one heavy-isotope label, which is suitable for absolute quantification or detection of a protein as described in WO/03016861, “Absolute Quantification of Proteins and Modified Forms Thereof by Multistage Mass Spectrometry” (Gygi et al.), further discussed below.

“Protein” is used interchangeably with polypeptide, and includes protein fragments and domains as well as whole protein.

“Acetylatable amino acid” means any amino acid that is capable of being modified by addition of a acetyl group, and includes both forms of such amino acid.

“Acetylatable peptide sequence” means a peptide sequence comprising an acetylatable amino acid.

“Acetylation site-specific antibody” means an antibody that specifically binds an acetylatable peptide sequence/epitope only when acetylated, or only when not acetylated, respectively. The term is used interchangeably with “acetyl-specific” antibody.

### A. Identification of Novel Protein Acetylation Protein Acetylation Sites.

The 432 novel Protein acetylation signaling protein acetylation sites disclosed herein and listed in Table 1/FIG. 2 were discovered by employing the modified peptide isolation and characterization techniques described in “Immunoaffinity Isolation of Modified Peptides From Complex Mixtures,” U.S. Patent Publication No. 20030044848, Rush et al. (the teaching of which is hereby incorporated herein by reference, in its entirety) using cellular extracts from the following human cancer-derived cell lines and patient samples: OCI/AML2, 293A, HepG2, HCT116, NB-4, OCI/AML3, SW620, sw480, HeLa and SIL-ALL. Acetyl-lysine specific antibodies were used in the Isolation and identification of acetylpeptides from these cell lines (Cell Signaling Technology, Inc., catalog number 9681) or a polyclonal anti-acetyl-lysine antibody (Cell Signaling Technology, Inc., catalog number 9441, purified bleed 7602, 7605, 7604). In addition to the 432 previously unknown protein acetylation sites (lysine) discovered, many known acetylation sites were also identified (not described herein). The immunoaffinity/mass spectrometric technique described in the '848 patent Publication (the “IAP” method)—and employed as described in detail in the Examples—is briefly summarized below.

The IAP method employed generally comprises the following steps: (a) a proteinaceous preparation (e.g. a digested cell extract) comprising acetylpeptides from two or more different proteins is obtained from an organism; (b) the preparation is contacted with at least one acetyl-lysine antibody (Cell Signaling Technology, Inc., catalog number 9681) or a polyclonal anti-acetyl-lysine antibody (Cell Signaling Technology, Inc., catalog number 9441, purified bleed 7602, 7605, 7604); (c) at least one acetylpeptide specifically bound by the immobilized antibody in step (b) is isolated; and (d) the modified peptide isolated in step (c) is characterized by mass spectrometry (MS) and/or tandem mass spectrometry (MS-MS). Subsequently, (e) a search program (e.g. Sequest) may be utilized to substantially match the spectra obtained for the isolated, modified peptide during the characterization of step (d) with the spectra for a known peptide sequence. A quantification step employing, e.g. SILAC or AQUA, may also be employed to quantify isolated peptides in order to compare peptide levels in a sample to a baseline.

In the IAP method as employed herein, at least one immobilized acetyl-lysine specific antibody (Cell Signaling Technology, Inc., catalog number 9681) or a polyclonal anti-acetyl-lysine antibody (Cell Signaling Technology, Inc., catalog number 9441, purified bleed 7602, 7605, 7604) was used in the immunoaffinity step to isolate the widest possible number of acetyl-lysine containing peptides from the cell extracts.

Extracts from the following cell lines were employed: OCI/AML2, 293A, HepG2, HCT116, NB-4, OCI/AML3, SW620, sw480, HeLa and SIL-ALL. These cells were treated with HDAC inhibitors (TSA and Nicotinamide).

As described in more detail in the Examples, lysates were prepared from these cells line and digested with trypsin after treatment with DTT and iodoacetamide to alkylate cysteine residues. Before the immunoaffinity step, peptides were pre-fractionated by reversed-phase solid phase extraction using Sep-Pak C₁₈ columns to separate peptides from other cellular components. The solid phase extraction cartridges were eluted with varying steps of acetonitrile. Each lyophilized peptide fraction was redissolved in MOP IP buffer and treated with an acetyl-lysine specific antibody (Cell Signaling Technology, Inc., catalog number 9681) or a polyclonal anti-acetyl-lysine antibody (Cell Signaling Technology, Inc., catalog number 9441, purified bleed 7602, 7605, 7604) immobilized on protein A-Sepharose or Protein A-Sepharose. Immunoaffinity-purified peptides were eluted with 0.15% TFA and a portion of this fraction was concentrated with Stage or Zip tips and analyzed by LC-MS/MS, using a ThermoFinnigan LCQ Deca XP Plus as well as LTQ ion trap mass spectrometer. Peptides were eluted from a 10 cm×75 μm reversed-phase column with a 45-min linear gradient of acetonitrile. MS/MS spectra were evaluated using the program Sequest with the NCBI human protein database.

This revealed a total of 432 novel lysine acetylation sites in protein acetylation signaling pathways. The identified acetylation sites and their parent proteins are enumerated in Table 1/FIG. 2. The lysine (human sequence) at which acetylation occurs is provided in Column D, and the peptide sequence encompassing the acetylatable lysine residue at the site is provided in Column E. FIG. 2 also shows the particular type of protein acetylation associated disease (see Column G) and cell line(s) (see Column F) in which a particular acetylation site was discovered.

As a result of the discovery of these acetylation sites, acetyl-specific antibodies and AQUA peptides for the detection of and quantification of these sites and their parent proteins may now be produced by standard methods, described below. These new reagents will prove highly useful in, e.g., studying the signaling pathways and events underlying the progression of protein acetylation associated diseases and the identification of new biomarkers and targets for diagnosis and treatment of such diseases.

### B. Antibodies and Cell Lines

Isolated acetylation site-specific antibodies that specifically bind a protein acetylation signaling protein disclosed in Column A of Table 1 only when acetylated (or only when not acetylated) at the corresponding amino acid and acetylation site listed in Columns D and E of Table 1/FIG. 2 may now be produced by standard antibody production methods, such as anti-peptide antibody methods, using the acetylation site sequence information provided in Column E of Table 1. For example, a previously unknown PARP1 DNA repair protein acetylation sites (lysine 105) (see Row 266 of Table 1/FIG. 2) are presently disclosed. Thus, an antibody that specifically binds novel PARP1 DNA repair protein sites can now be produced, e.g. by immunizing an animal with a peptide antigen comprising all or part of the amino acid sequence encompassing the respective acetylated residue (e.g. a peptide antigen comprising the sequence set forth in Row 266, Column E, of Table 1 (SEQ ID NO: 265) (which encompasses the acetylated lysine at position 105 in PARP1), to produce an antibody that only binds PARP1 DNA repair protein when acetylated at that site.

Polyclonal antibodies of the invention may be produced according to standard techniques by immunizing a suitable animal (e.g., rabbit, goat, etc.) with a peptide antigen corresponding to the protein acetylation acetylation site of interest (i.e. a acetylation site enumerated in Column E of Table 1, which comprises the corresponding acetylatable amino acid listed in Column D of Table 1), collecting immune serum from the animal, and separating the polyclonal antibodies from the immune serum, in accordance with known procedures. For example, a peptide antigen corresponding to all or part of the novel MRVI1 Adaptor/Scaffold acetylation site disclosed herein (SEQ ID NO: 38=FAGKAGGKLAKAPGLK, encompassing acetylated lysine 405 (see Row 39 of Table 1)) may be used to produce antibodies that only bind MRVI1 when acetylated at Lys405. Similarly, a peptide comprising all or part of any one of the acetylation site sequences provided in Column E of Table 1 may employed as an antigen to produce an antibody that only binds the corresponding protein listed in Column A of Table 1 when acetylated (or when not acetylated) at the corresponding residue listed in Column D. If an antibody that only binds the protein when acetylated at the disclosed site is desired, the peptide antigen includes the acetylated form of the amino acid. Conversely, if an antibody that only binds the protein when not acetylated at the disclosed site is desired, the peptide antigen includes the non-acetylated form of the amino acid.

Peptide antigens suitable for producing antibodies of the invention may be designed, constructed and employed in accordance with well-known techniques. See, e.g., ANTIBODIES: A LABORATORY MANUAL, Chapter 5, p. 75-76, Harlow & Lane Eds., Cold Spring Harbor Laboratory (1988); Czernik, Methods In Enzymology, 201: 264-283 (1991); Merrifield, J. Am. Chem. Soc. 85:21-49 (1962)).

It will be appreciated by those of skill in the art that longer or shorter acetylpeptide antigens may be employed. See Id. For example, a peptide antigen may comprise the full sequence disclosed in Column E of Table 1/FIG. 2, or it may comprise additional amino acids flanking such disclosed sequence, or may comprise of only a portion of the disclosed sequence immediately flanking the acetylatable amino acid (indicated in Column E by uppercase “K”). Typically, a desirable peptide antigen will comprise four or more amino acids flanking each side of the acetylatable amino acid and encompassing it. Polyclonal antibodies produced as described herein may be screened as further described below.

Monoclonal antibodies of the invention may be produced in a hybridoma cell line according to the well-known technique of Kohler and Milstein. See Nature 265:495-97 (1975); Kohler and Milstein, Eur. J. Immunol. 6: 511 (1976); see also, CURRENT PROTOCOLS IN MOLECULAR BIOLOGY, Ausubel et al. Eds. (1989). Monoclonal antibodies so produced are highly specific, and improve the selectivity and specificity of diagnostic assay methods provided by the invention. For example, a solution containing the appropriate antigen may be injected into a mouse or other species and, after a sufficient time (in keeping with conventional techniques), the animal is sacrificed and spleen cells obtained. The spleen cells are then immortalized by fusing them with myeloma cells, typically in the presence of polyethylene glycol, to produce hybridoma cells. Rabbit fusion hybridomas, for example, may be produced as described in U.S. Pat. No. 5,675,063, C. Knight, Issued Oct. 7, 1997. The hybridoma cells are then grown in a suitable selection media, such as hypoxanthine-aminopterin-thymidine (HAT), and the supernatant screened for monoclonal antibodies having the desired specificity, as described below. The secreted antibody may be recovered from tissue culture supernatant by conventional methods such as precipitation, ion exchange or affinity chromatography, or the like.

Monoclonal Fab fragments may also be produced in Escherichia coli by recombinant techniques known to those skilled in the art. See, e.g., W. Huse, Science 246:1275-81 (1989); Mullinax et al., Proc. Nat'l Acad. Sci. 87: 8095 (1990). If monoclonal antibodies of one isotype are preferred for a particular application, particular isotypes can be prepared directly, by selecting from the initial fusion, or prepared secondarily, from a parental hybridoma secreting a monoclonal antibody of different isotype by using the sib selection technique to isolate class-switch variants (Steplewski, et al., Proc. Nat'l. Acad. Sci., 82: 8653 (1985); Spira et al., J. Immunol. Methods, 74: 307 (1984)).

The preferred epitope of a acetylation-site specific antibody of the invention is a peptide fragment consisting essentially of about 8 to 17 amino acids including the acetylatable lysine, wherein about 3 to 8 amino acids are positioned on each side of the acetylatable lysine (for example, the ALS2CR19 lysine 848 acetylation site sequence disclosed in Row 27, Column E of Table 1), and antibodies of the invention thus specifically bind a target Protein acetylation signaling polypeptide comprising such epitopic sequence. Particularly preferred epitopes bound by the antibodies of the invention comprise all or part of an acetylatable site sequence listed in Column E of Table 1, including the acetylatable amino acid.

Included in the scope of the invention are equivalent non-antibody molecules, such as protein binding domains or nucleic acid aptamers, which bind, in a acetyl-specific manner, to essentially the same acetylatable epitope to which the acetyl-specific antibodies of the invention bind. See, e.g., Neuberger et al., Nature 312:604 (1984). Such equivalent non-antibody reagents may be suitably employed in the methods of the invention further described below.

Antibodies provided by the invention may be any type of immunoglobulins, including IgG, IgM, IgA, IgD, and IgE, including F_{ab} or antigen-recognition fragments thereof. The antibodies may be monoclonal or polyclonal and may be of any species of origin, including (for example) mouse, rat, rabbit, horse, or human, or may be chimeric antibodies. See, e.g., M. Walker et al., Molec. Immunol. 26: 403-11 (1989); Morrision et al., Proc. Nat'l. Acad. Sci. 81: 6851 (1984); Neuberger et al., Nature 312:604 (1984)). The antibodies may be recombinant monoclonal antibodies produced according to the methods disclosed in U.S. Pat. No. 4,474,893 (Reading) or U.S. Pat. No. 4,816,567 (Cabilly et al.) The antibodies may also be chemically constructed by specific antibodies made according to the method disclosed in U.S. Pat. No. 4,676,980 (Segel et al.)

The invention also provides immortalized cell lines that produce an antibody of the invention. For example, hybridoma clones, constructed as described above, that produce monoclonal antibodies to the protein acetylation signaling protein acetylation sties disclosed herein are also provided. Similarly, the invention includes recombinant cells producing an antibody of the invention, which cells may be constructed by well known techniques; for example the antigen combining site of the monoclonal antibody can be cloned by PCR and single-chain antibodies produced as phage-displayed recombinant antibodies or soluble antibodies in E. coli (see, e.g., ANTIBODY ENGINEERING PROTOCOLS, 1995, Humana Press, Sudhir Paul editor.)

Acetylation site-specific antibodies of the invention, whether polyclonal or monoclonal, may be screened for epitope and acetyl-specificity according to standard techniques. See, e.g. Czernik et al., Methods in Enzymology, 201: 264-283 (1991). For example, the antibodies may be screened against the acetyl and non-acetyl peptide library by ELISA to ensure specificity for both the desired antigen (i.e. that epitope including a acetylation site sequence enumerated in Column E of Table 1) and for reactivity only with the acetylated (or non-acetylated) form of the antigen. Peptide competition assays may be carried out to confirm lack of reactivity with other acetyl-epitopes on the given protein acetylation signaling protein. The antibodies may also be tested by Western blotting against cell preparations containing the signaling protein, e.g. cell lines over-expressing the target protein, to confirm reactivity with the desired acetylated epitope/target.

Specificity against the desired acetylated epitope may also be examined by constructing mutants lacking acetylatable residues at positions outside the desired epitope that are known to be acetylated, or by mutating the desired acetyl-epitope and confirming lack of reactivity. Acetylation-site specific antibodies of the invention may exhibit some limited cross-reactivity to related epitopes in non-target proteins. This is not unexpected as most antibodies exhibit some degree of cross-reactivity, and anti-peptide antibodies will often cross-react with epitopes having high homology to the immunizing peptide. See, e.g., Czernik, supra. Cross-reactivity with non-target proteins is readily characterized by Western blotting alongside markers of known molecular weight. Amino acid sequences of cross-reacting proteins may be examined to identify sites highly homologous to the protein acetylation signaling protein epitope for which the antibody of the invention is specific.

In certain cases, polyclonal antisera may exhibit some undesirable general cross-reactivity to acetyl-lysine itself, which may be removed by further purification of antisera, e.g. over an acetyltyramine column. Antibodies of the invention specifically bind their target protein (i.e. a protein listed in Column A of Table 1) only when acetylated (or only when not acetylated, as the case may be) at the site disclosed in corresponding Columns D/E, and do not (substantially) bind to the other form (as compared to the form for which the antibody is specific).

Antibodies may be further characterized via immunohistochemical (IHC) staining using normal and diseased tissues to examine protein acetylation and activation status in diseased tissue. IHC may be carried out according to well-known techniques. See, e.g., ANTIBODIES: A LABORATORY MANUAL, Chapter 10, Harlow & Lane Eds., Cold Spring Harbor Laboratory (1988). Briefly, paraffin-embedded tissue (e.g. tumor tissue) is prepared for immunohistochemical staining by deparaffinizing tissue sections with xylene followed by ethanol; hydrating in water then PBS; unmasking antigen by heating slide in sodium citrate buffer; incubating sections in hydrogen peroxide; blocking in blocking solution; incubating slide in primary antibody and secondary antibody; and finally detecting using ABC avidin/biotin method according to manufacturer's instructions.

Antibodies may be further characterized by flow cytometry carried out according to standard methods. See Chow et al., Cytometry (Communications in Clinical Cytometry) 46:7265-274 (2001). Briefly and by way of example, the following protocol for cytometric analysis may be employed: samples may be centrifuged on Ficoll gradients to remove erythrocytes, and cells may then be fixed with 2% paraformaldehyde for 10 minutes at 37° C. followed by permeabilization in 90% methanol for 30 minutes on ice. Cells may then be stained with the primary acetylation-site specific antibody of the invention (which detects a protein acetylation signal transduction protein enumerated in Table 1), washed and labeled with a fluorescent-labeled secondary antibody. Additional fluorochrome-conjugated marker antibodies (e.g. CD45, CD34) may also be added at this time to aid in the subsequent identification of specific hematopoietic cell types. The cells would then be analyzed on a flow cytometer (e.g. a Beckman Coulter FC500) according to the specific protocols of the instrument used.

Antibodies of the invention may also be advantageously conjugated to fluorescent dyes (e.g. Alexa488, PE) for use in multi-parametric analyses along with other signal transduction (acetyl-CrkL, acetyl-Erk 1/2) and/or cell marker (CD34) antibodies.

Acetylation-site specific antibodies of the invention specifically bind to a human protein acetylation signal transduction protein or polypeptide only when acetylated at a disclosed site, but are not limited only to binding the human species, per se. The invention includes antibodies that also bind conserved and highly homologous or identical acetylation sites in respective protein acetylation proteins from other species (e.g. mouse, rat, monkey, yeast), in addition to binding the human acetylation site. Highly homologous or identical sites conserved in other species can readily be identified by standard sequence comparisons, such as using BLAST, with the human protein acetylation signal transduction protein acetylation sites disclosed herein.

### C. Heavy-Isotope Labeled Peptides (AQUA Peptides).

The novel protein acetylation signaling protein acetylation sites disclosed herein now enable the production of corresponding heavy-isotope labeled peptides for the absolute quantification of such signaling proteins (both acetylated and not acetylated at a disclosed site) in biological samples. The production and use of AQUA peptides for the absolute quantification of proteins (AQUA) in complex mixtures has been described. See WO/03016861, “Absolute Quantification of Proteins and Modified Forms Thereof by Multistage Mass Spectrometry,” Gygi et al. and also Gerber et al. Proc. Natl. Acad. Sci. U.S.A. 100: 6940-5 (2003) (the teachings of which are hereby incorporated herein by reference, in their entirety).

The AQUA methodology employs the introduction of a known quantity of at least one heavy-isotope labeled peptide standard (which has a unique signature detectable by LC-SRM chromatography) into a digested biological sample in order to determine, by comparison to the peptide standard, the absolute quantity of a peptide with the same sequence and protein modification in the biological sample. Briefly, the AQUA methodology has two stages: peptide internal standard selection and validation and method development; and implementation using validated peptide internal standards to detect and quantify a target protein in sample. The method is a powerful technique for detecting and quantifying a given peptide/protein within a complex biological mixture, such as a cell lysate, and may be employed, e.g., to quantify change in protein acetylation as a result of drug treatment, or to quantify differences in the level of a protein in different biological states.

Generally, to develop a suitable internal standard, a particular peptide (or modified peptide) within a target protein sequence is chosen based on its amino acid sequence and the particular protease to be used to digest. The peptide is then generated by solid-phase peptide synthesis such that one residue is replaced with that same residue containing stable isotopes (¹³C, ¹⁵N). The result is a peptide that is chemically identical to its native counterpart formed by proteolysis, but is easily distinguishable by MS via a 7-Da mass shift. A newly synthesized AQUA internal standard peptide is then evaluated by LC-MS/MS. This process provides qualitative information about peptide retention by reverse-phase chromatography, ionization efficiency, and fragmentation via collision-induced dissociation. Informative and abundant fragment ions for sets of native and internal standard peptides are chosen and then specifically monitored in rapid succession as a function of chromatographic retention to form a selected reaction monitoring (LC-SRM) method based on the unique profile of the peptide standard.

The second stage of the AQUA strategy is its implementation to measure the amount of a protein or modified protein from complex mixtures. Whole cell lysates are typically fractionated by SDS-PAGE gel electrophoresis, and regions of the gel consistent with protein migration are excised. This process is followed by in-gel proteolysis in the presence of the AQUA peptides and LC-SRM analysis. (See Gerber et al. supra.) AQUA peptides are spiked in to the complex peptide mixture obtained by digestion of the whole cell lysate with a proteolytic enzyme and subjected to immunoaffinity purification as described above. The retention time and fragmentation pattern of the native peptide formed by digestion (e.g. trypsinization) is identical to that of the AQUA internal standard peptide determined previously; thus, LC-MS/MS analysis using an SRM experiment results in the highly specific and sensitive measurement of both internal standard and analyte directly from extremely complex peptide mixtures. Because an absolute amount of the AQUA peptide is added (e.g. 250 fmol), the ratio of the areas under the curve can be used to determine the precise expression levels of a protein or acetylated form of a protein in the original cell lysate. In addition, the internal standard is present during in-gel digestion as native peptides are formed, such that peptide extraction efficiency from gel pieces, absolute losses during sample handling (including vacuum centrifugation), and variability during introduction into the LC-MS system do not affect the determined ratio of native and AQUA peptide abundances.

An AQUA peptide standard is developed for a known acetylation site sequence previously identified by the IAP-LC-MS/MS method within a target protein. One AQUA peptide incorporating the acetylated form of the particular residue within the site may be developed, and a second AQUA peptide incorporating the non-acetylated form of the residue developed. In this way, the two standards may be used to detect and quantify both the acetylated and non-acetylated forms of the site in a biological sample.

Peptide internal standards may also be generated by examining the primary amino acid sequence of a protein and determining the boundaries of peptides produced by protease cleavage. Alternatively, a protein may actually be digested with a protease and a particular peptide fragment produced can then sequenced. Suitable proteases include, but are not limited to, lysine proteases (e.g. trypsin, hepsin), metallo proteases (e.g. PUMP1), chymotrypsin, cathepsin, pepsin, thermolysin, carboxypeptidases, etc.

A peptide sequence within a target protein is selected according to one or more criteria to optimize the use of the peptide as an internal standard. Preferably, the size of the peptide is selected to minimize the chances that the peptide sequence will be repeated elsewhere in other non-target proteins. Thus, a peptide is preferably at least about 6 amino acids. The size of the peptide is also optimized to maximize ionization frequency. Thus, peptides longer than about 20 amino acids are not preferred. The preferred ranged is about 7 to 15 amino acids. A peptide sequence is also selected that is not likely to be chemically reactive during mass spectrometry, thus sequences comprising cysteine, tryptophan, or methionine are avoided.

A peptide sequence that does not include a modified region of the target region may be selected so that the peptide internal standard can be used to determine the quantity of all forms of the protein. Alternatively, a peptide internal standard encompassing a modified amino acid may be desirable to detect and quantify only the modified form of the target protein. Peptide standards for both modified and unmodified regions can be used together, to determine the extent of a modification in a particular sample (i.e. to determine what fraction of the total amount of protein is represented by the modified form). For example, peptide standards for both the acetylated and unacetylated form of a protein known to be acetylated at a particular site can be used to quantify the amount of acetylated form in a sample.

The peptide is labeled using one or more labeled amino acids (i.e. the label is an actual part of the peptide) or less preferably, labels may be attached after synthesis according to standard methods. Preferably, the label is a mass-altering label selected based on the following considerations: The mass should be unique to shift fragment masses produced by MS analysis to regions of the spectrum with low background; the ion mass signature component is the portion of the labeling moiety that preferably exhibits a unique ion mass signature in MS analysis; the sum of the masses of the constituent atoms of the label is preferably uniquely different than the fragments of all the possible amino acids. As a result, the labeled amino acids and peptides are readily distinguished from unlabeled ones by the ion/mass pattern in the resulting mass spectrum. Preferably, the ion mass signature component imparts a mass to a protein fragment that does not match the residue mass for any of the natural amino acids.

The label should be robust under the fragmentation conditions of MS and not undergo unfavorable fragmentation. Labeling chemistry should be efficient under a range of conditions, particularly denaturing conditions, and the labeled tag preferably remains soluble in the MS buffer system of choice. The label preferably does not suppress the ionization efficiency of the protein and is not chemically reactive. The label may contain a mixture of two or more isotopically distinct species to generate a unique mass spectrometric pattern at each labeled fragment position. Stable isotopes, such as ²H, ¹³C, ¹⁵N, ¹⁷O, ¹⁸O, or ³⁴S, are among preferred labels. Pairs of peptide internal standards that incorporate a different isotope label may also be prepared. Preferred amino acid residues into which a heavy isotope label may be incorporated include leucine, proline, valine, and phenylalanine.

Peptide internal standards are characterized according to their mass-to-charge (m/z) ratio, and preferably, also according to their retention time on a chromatographic column (e.g. an HPLC column). Internal standards that co-elute with unlabeled peptides of identical sequence are selected as optimal internal standards. The internal standard is then analyzed by fragmenting the peptide by any suitable means, for example by collision-induced dissociation (CID) using, e.g., argon or helium as a collision gas. The fragments are then analyzed, for example by multi-stage mass spectrometry (MSⁿ) to obtain a fragment ion spectrum, to obtain a peptide fragmentation signature. Preferably, peptide fragments have significant differences in m/z ratios to enable peaks corresponding to each fragment to be well separated, and a signature that is unique for the target peptide is obtained. If a suitable fragment signature is not obtained at the first stage, additional stages of MS are performed until a unique signature is obtained.

Fragment ions in the MS/MS and MS³ spectra are typically highly specific for the peptide of interest, and, in conjunction with LC methods, allow a highly selective means of detecting and quantifying a target peptide/protein in a complex protein mixture, such as a cell lysate, containing many thousands or tens of thousands of proteins. Any biological sample potentially containing a target protein/peptide of interest may be assayed. Crude or partially purified cell extracts are preferably employed. Generally, the sample has at least 0.01 mg of protein, typically a concentration of 0.1-10 mg/mL, and may be adjusted to a desired buffer concentration and pH.

A known amount of a labeled peptide internal standard, preferably about 10 femtomoles, corresponding to a target protein to be detected/quantified is then added to a biological sample, such as a cell lysate. The spiked sample is then digested with one or more protease(s) for a suitable time period to allow digestion. A separation is then performed (e.g. by HPLC, reverse-phase HPLC, capillary electrophoresis, ion exchange chromatography, etc.) to isolate the labeled internal standard and its corresponding target peptide from other peptides in the sample. Microcapillary LC is a preferred method.

Each isolated peptide is then examined by monitoring of a selected reaction in the MS. This involves using the prior knowledge gained by the characterization of the peptide internal standard and then requiring the MS to continuously monitor a specific ion in the MS/MS or MSⁿ spectrum for both the peptide of interest and the internal standard. After elution, the area under the curve (AUC) for both peptide standard and target peptide peaks are calculated. The ratio of the two areas provides the absolute quantification that can be normalized for the number of cells used in the analysis and the protein's molecular weight, to provide the precise number of copies of the protein per cell. Further details of the AQUA methodology are described in Gygi et al., and Gerber et al. supra.

In accordance with the present invention, AQUA internal peptide standards (heavy-isotope labeled peptides) may now be produced, as described above, for any of the 432 novel Protein acetylation signaling protein acetylation sites disclosed herein (see Table 1/FIG. 2). Peptide standards for a given acetylation site (e.g. the lysine 803 in DSP—see Row 61 of Table 1) may be produced for both the acetylated and non-acetylated forms of the site (e.g. see DSP site sequence in Column E, Row 61 of Table 1 (SEQ ID NO: 60) and such standards employed in the AQUA methodology to detect and quantify both forms of such acetylation site in a biological sample.

AQUA peptides of the invention may comprise all, or part of, an acetylation site peptide sequence disclosed herein (see Column E of Table 1/FIG. 2). In a preferred embodiment, an AQUA peptide of the invention comprises an acetylation site sequence disclosed herein in Table 1/FIG. 2. For example, an AQUA peptide of the invention for detection/quantification of ATRX DNA repair protein when acetylated at lysine K1935 may comprise the sequence KKKKGkKGKK (k=acetyl-lysine), which comprises acetylatable lysine 1935 (see Row 271, Column E; (SEQ ID NO: 270)). Heavy-isotope labeled equivalents of the peptides enumerated in Table 1/FIG. 2 (both in acetylated and unacetylated form) can be readily synthesized and their unique MS and LC-SRM signature determined, so that the peptides are validated as AQUA peptides and ready for use in quantification experiments.

The acetylation site peptide sequences disclosed herein (see Column E of Table 1/FIG. 2) are particularly well suited for development of corresponding AQUA peptides, since the IAP method by which they were identified (see Part A above and Example 1) inherently confirmed that such peptides are in fact produced by enzymatic digestion (trypsinization) and are in fact suitably fractionated/ionized in MS/MS. Thus, heavy-isotope labeled equivalents of these peptides (both in acetylated and unacetylated form) can be readily synthesized and their unique MS and LC-SRM signature determined, so that the peptides are validated as AQUA peptides and ready for use in quantification experiments.

Accordingly, the invention provides heavy-isotope labeled peptides (AQUA peptides) for the detection and/or quantification of any of the protein acetylation sites disclosed in Table 1/FIG. 2 (see Column E) and/or their corresponding parent proteins/polypeptides (see Column A). An acetyl peptide sequence comprising any of the acetylation sequences listed in Table 1 may be considered a preferred AQUA peptide of the invention. For example, an AQUA peptide comprising the sequence MLPYkVTQDELK (SEQ ID NO: 257) (where k is acetyl-lysine, and where V=labeled valine (e.g. ¹⁴C)) is provided for the quantification of acetylated (or non-acetylated) NCL DNA binding protein (Lys403) in a biological sample (see Row 258 of Table 1, lysine 403 being the acetylatable residue within the site). However, it will be appreciated that a larger AQUA peptide comprising a disclosed acetylation site sequence (and additional residues downstream or upstream of it) may also be constructed. Similarly, a smaller AQUA peptide comprising less than all of the residues of a disclosed acetylation site sequence (but still comprising the acetylatable residue enumerated in Column D of Table 1/FIG. 2) may alternatively be constructed. Such larger or shorter AQUA peptides are within the scope of the present invention, and the selection and production of preferred AQUA peptides may be carried out as described above (see Gygi et al., Gerber et al. supra.).

Certain particularly preferred subsets of AQUA peptides provided by the invention are described above (corresponding to particular protein types/groups in Table 1, for example, Acetyltransferases and DNA repair proteins). Example 4 is provided to further illustrate the construction and use, by standard methods described above, of exemplary AQUA peptides provided by the invention. For example, the above-described AQUA peptides corresponding to both the acetylated and non-acetylated forms of the disclosed ATP5A1 Enzyme protein lysine, 498 acetylation site (see Row 295 of Table 1/FIG. 2) may be used to quantify the amount of acetylated WNK1 kinase (Lys498) in a biological sample, e.g. a tumor cell sample (or a sample before or after treatment with a test drug).

AQUA peptides of the invention may also be employed within a kit that comprises one or multiple AQUA peptide(s) provided herein (for the quantification of a Protein acetylation signal transduction protein disclosed in Table 1/FIG. 2), and, optionally, a second detecting reagent conjugated to a detectable group. For example, a kit may include AQUA peptides for both the acetylated and non-acetylated form of an acetylation site disclosed herein. The reagents may also include ancillary agents such as buffering agents and protein stabilizing agents, e.g., polysaccharides and the like. The kit may further include, where necessary, other members of the signal-producing system of which system the detectable group is a member (e.g., enzyme substrates), agents for reducing background interference in a test, control reagents, apparatus for conducting a test, and the like. The test kit may be packaged in any suitable manner, typically with all elements in a single container along with a sheet of printed instructions for carrying out the test.

AQUA peptides provided by the invention will be highly useful in the further study of signal transduction anomalies underlying cancer, including both solid and blood borne cancers, and in identifying diagnostic/bio-markers of these diseases, new potential drug targets, and/or in monitoring the effects of test compounds on protein acetylation signal transduction proteins and pathways.

### D. Immunoassay Formats

Antibodies provided by the invention may be advantageously employed in a variety of standard immunological assays (the use of AQUA peptides provided by the invention is described separately above). Assays may be homogeneous assays or heterogeneous assays. In a homogeneous assay the immunological reaction usually involves a acetylation-site specific antibody of the invention), a labeled analyte, and the sample of interest. The signal arising from the label is modified, directly or indirectly, upon the binding of the antibody to the labeled analyte. Both the immunological reaction and detection of the extent thereof are carried out in a homogeneous solution. Immunochemical labels that may be employed include free radicals, radioisotopes, fluorescent dyes, enzymes, bacteriophages, coenzymes, and so forth.

In a heterogeneous assay approach, the reagents are usually the specimen, an acetylation-site specific antibody of the invention, and suitable means for producing a detectable signal. Similar specimens as described above may be used. The antibody is generally immobilized on a support, such as a bead, plate or slide, and contacted with the specimen suspected of containing the antigen in a liquid phase. The support is then separated from the liquid phase and either the support phase or the liquid phase is examined for a detectable signal employing means for producing such signal. The signal is related to the presence of the analyte in the specimen. Means for producing a detectable signal include the use of radioactive labels, fluorescent labels, enzyme labels, and so forth. For example, if the antigen to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays are the radioimmunoassay, immunofluorescence methods, enzyme-linked immunoassays, and the like.

Immunoassay formats and variations thereof that may be useful for carrying out the methods disclosed herein are well known in the art. See generally E. Maggio, Enzyme-Immunoassay, (1980) (CRC Press, Inc., Boca Raton, Fla.); see also, e.g., U.S. Pat. No. 4,727,022 (Skold et al., “Methods for Modulating Ligand-Receptor Interactions and their Application”); U.S. Pat. No. 4,659,678 (Forrest et al., “Immunoassay of Antigens”); U.S. Pat. No. 4,376,110 (David et al., “Immunometric Assays Using Monoclonal Antibodies”). Conditions suitable for the formation of reagent-antibody complexes are well described. See id. Monoclonal antibodies of the invention may be used in a “two-site” or “sandwich” assay, with a single cell line serving as a source for both the labeled monoclonal antibody and the bound monoclonal antibody. Such assays are described in U.S. Pat. No. 4,376,110. The concentration of detectable reagent should be sufficient such that the binding of a target Protein acetylation signal transduction protein is detectable compared to background.

Acetylation site-specific antibodies disclosed herein may be conjugated to a solid support suitable for a diagnostic assay (e.g., beads, plates, slides or wells formed from materials such as latex or polystyrene) in accordance with known techniques, such as precipitation. Antibodies, or other target protein or target site-binding reagents, may likewise be conjugated to detectable groups such as radiolabels (e.g., ³⁵S, ¹²⁵I, ¹³¹I), enzyme labels (e.g., horseradish peroxidase, alkaline phosphatase), and fluorescent labels (e.g., fluorescein) in accordance with known techniques.

Antibodies of the invention may also be optimized for use in a flow cytometry (FC) assay to determine the activation/acetylation status of a target Protein acetylation signal transduction protein in patients before, during, and after treatment with a drug targeted at inhibiting acetylation at such a protein at the acetylation site disclosed herein. For example, bone marrow cells or peripheral blood cells from patients may be analyzed by flow cytometry for target Protein acetylation signal transduction protein acetylation, as well as for markers identifying various hematopoietic cell types. In this manner, activation status of the malignant cells may be specifically characterized. Flow cytometry may be carried out according to standard methods. See, e.g. Chow et al., Cytometry (Communications in Clinical Cytometry) 46:72-78 (2001). Briefly and by way of example, the following protocol for cytometric analysis may be employed: fixation of the cells with 1% para-formaldehyde for 10 minutes at 37° C. followed by permeabilization in 90% methanol for 30 minutes on ice. Cells may then be stained with the primary antibody (an acetyl-specific antibody of the invention), washed and labeled with a fluorescent-labeled secondary antibody. Alternatively, the cells may be stained with a fluorescent-labeled primary antibody. The cells would then be analyzed on a flow cytometer (e.g. a Beckman Coulter EPICS-XL) according to the specific protocols of the instrument used. Such an analysis would identify the presence of activated protein acetylation signal transduction protein(s) in the malignant cells and reveal the drug response on the targeted protein.

Alternatively, antibodies of the invention may be employed in immunohistochemical (IHC) staining to detect differences in signal transduction or protein activity using normal and diseased tissues. IHC may be carried out according to well-known techniques. See, e.g., ANTIBODIES: A LABORATORY MANUAL, supra. Briefly, paraffin-embedded tissue (e.g. tumor tissue) is prepared for immunohistochemical staining by deparaffinizing tissue sections with xylene followed by ethanol; hydrating in water then PBS; unmasking antigen by heating slide in sodium citrate buffer; incubating sections in hydrogen peroxide; blocking in blocking solution; incubating slide in primary antibody and secondary antibody; and finally detecting using ABC avidin/biotin method according to manufacturer's instructions.

Antibodies of the invention may be also be optimized for use in other clinically-suitable applications, for example bead-based multiplex-type assays, such as IGEN, Luminex™ and/or Bioplex™ assay formats, or otherwise optimized for antibody arrays formats, such as reversed-phase array applications (see, e.g. Paweletz et al., Oncogene 20(16): 1981-89 (2001)). Accordingly, in another embodiment, the invention provides a method for the multiplex detection of protein acetylation in a biological sample, the method comprising utilizing two or more antibodies or AQUA peptides of the invention to detect the presence of two or more acetylated protein acetylation signaling proteins enumerated in Column A of Table 1/FIG. 2. In one preferred embodiment, two to five antibodies or AQUA peptides of the invention are employed in the method. In another preferred embodiment, six to ten antibodies or AQUA peptides of the invention are employed, while in another preferred embodiment eleven to twenty such reagents are employed.

Antibodies and/or AQUA peptides of the invention may also be employed within a kit that comprises at least one acetylation site-specific antibody or AQUA peptide of the invention (which binds to or detects a Protein acetylation signal transduction protein disclosed in Table 1/FIG. 2), and, optionally, a second antibody conjugated to a detectable group. In some embodiments, the kit is suitable for multiplex assays and comprises two or more antibodies or AQUA peptides of the invention, and in some embodiments, comprises two to five, six to ten, or eleven to twenty reagents of the invention. The kit may also include ancillary agents such as buffering agents and protein stabilizing agents, e.g., polysaccharides and the like. The kit may further include, where necessary, other members of the signal-producing system of which system the detectable group is a member (e.g., enzyme substrates), agents for reducing background interference in a test, control reagents, apparatus for conducting a test, and the like. The test kit may be packaged in any suitable manner, typically with all elements in a single container along with a sheet of printed instructions for carrying out the test.

The following Examples are provided only to further illustrate the invention, and are not intended to limit its scope, except as provided in the claims appended hereto. The present invention encompasses modifications and variations of the methods taught herein which would be obvious to one of ordinary skill in the art.

### EXAMPLE 1

### Isolation of Acetyl-Lysine Containing Peptides from Extracts of Human Cancer Cell Lines and Identification of Novel Acetylation Sites

In order to discover previously unknown protein acetylation signal transduction protein acetylation sites, IAP isolation techniques were employed to identify acetyl-lysine containing peptides in cell extracts from the following cell lines: OCI/AML2, 293A, HepG2, HCT116, NB-4, OCI/AML3, SW620, sw480, HeLa and SIL-ALL. OCI/AMLL2, OCI/AML3, NB-4, and SIL-ALL cell lines were grown in RPMI1640 medium with 10% FBS. 293A, HepG2, and HeLa cells were grown in MEM medium with 10% FBS. HCT116, SW620, and sw480 cells were grown in DMEM medium with 10% FBS. Cells were either untreated or treated with HDAC inhibitors TSA or Nicotinamide, were harvested when they were about 60-80% confluent. About 200 million cells were harvested in 10 mL lysis buffer per 2×10⁸ cells (20 mM HEPES pH 8.0, 9 M urea, 1 mM sodium vanadate, supplemented with 2.5 mM sodium pyro-phosphate, 1 mM 9-glycerol-phosphate) and sonicated.

Sonicated cell lysates were cleared by centrifugation at 20,000×g, and proteins were reduced with DTT at a final concentration of 4.1 mM and alkylated with iodoacetamide at 8.3 mM. For digestion with trypsin, protein extracts were diluted in 20 mM HEPES pH 8.0 to a final concentration of 2 M urea and soluble TPCK-trypsin (Worthington) was added at 10-20 μg/mL. Digestion was performed for overnight at room temperature.

Trifluoroacetic acid (TFA) was added to protein digests to a final concentration of 1%, precipitate was removed by centrifugation, and digests were loaded onto Sep-Pak C₁₈ columns (Waters) equilibrated with 0.1% TFA. A column volume of 0.7-1.0 ml was used per 2×10⁸ cells. Columns were washed with 15 volumes of 0.1% TFA, followed by 4 volumes of 5% acetonitrile (MeCN) in 0.1% TFA. Bound peptide was eluted with step-wise increasing concentration of acetonitrile (85, 12%, 15%, 18%, 22%, 25%, 30%, 35%, 40%) in 0.1% TFA. Peptide elute was then lyophilized.

Lyophilized peptide was dissolved in 1.4 ml of IAP buffer (20 mM Tris/HCl or 50 mM MOPS pH 7.2, 10 mM sodium phosphate, 50 mM NaCl) and insoluble matter was removed by centrifugation. The monoclonal anti-acetyl-lysine antibody (Cell Signaling Technology, Inc., catalog number 9681) or a polyclonal anti-acetyl-lysine antibody (Cell Signaling Technology, Inc., catalog number 9441, purified bleed 7602, 7605, 7604) was coupled at 4 mg/ml beads to protein G or protein A agarose (Roche), respectively. Immobilized antibody (40 μl, 160 μg) was added as 1:1 slurry in IAP buffer to 1.4 ml of cleared peptide solution, and the mixture was incubated overnight at 4° C. with gentle rotation. The immobilized antibody beads were washed three times with 1 ml IAP buffer and twice with 1 ml water, all at 4° C. Peptides were eluted from beads by incubation with 55 μl of 0.15% TFA at room temperature for 10 min (eluate 1), followed by a wash of the beads (eluate 2) with 45 μl of 0.15% TFA. Both eluates were combined.

### Analysis by LC-MS/MS Mass Spectrometry

40 μl or more of IAP eluate were purified by 0.2 μl StageTips or ZipTips. Peptides were eluted from the microcolumns with 1 μl of 40% MeCN, 0.1% TFA (fractions I and II) or 1 μl of 60% MeCN, 0.1% TFA (fraction III) into 7.6 μl of 0.4% acetic acid/0.005% heptafluorobutyric acid. This sample was loaded onto a 10 cm×75 μm PicoFrit capillary column (New Objective) packed with Magic C18 AQ reversed-phase resin (Michrom Bioresources) using a Famos autosampler with an inert sample injection valve (Dionex). The column was then developed with a 45-min linear gradient of acetonitrile delivered at 200 nl/min (Ultimate, Dionex), and tandem mass spectra were collected in a data-dependent manner with an LCQ Deca XP Plus ion trap mass spectrometer essentially as described by Gygi et al., supra.

### Database Analysis & Assignments.

MS/MS spectra were evaluated using TurboSequest in the Sequest Browser package (v. 27, rev. 12) supplied as part of BioWorks 3.0 (ThermoFinnigan). Individual MS/MS spectra were extracted from the raw data file using the Sequest Browser program CreateDta, with the following settings: bottom MW, 700; top MW, 4,500; minimum number of ions, 20; minimum TIC, 4×10⁵; and precursor charge state, unspecified. Spectra were extracted from the beginning of the raw data file before sample injection to the end of the eluting gradient. The IonQuest and VuDta programs were not used to further select MS/MS spectra for Sequest analysis. MS/MS spectra were evaluated with the following TurboSequest parameters: peptide mass tolerance, 2.5; fragment ion tolerance, 0.0; maximum number of differential amino acids per modification, 4; mass type parent, average; mass type fragment, average; maximum number of internal cleavage sites, 10; neutral losses of water and ammonia from b and y ions were considered in the correlation analysis. Proteolytic enzyme was specified except for spectra collected from elastase digests.

Searches were performed against the NCBI human protein database (as released on Aug. 24, 2004 and containing 27, 960 protein sequences). Cysteine carboxamidomethylation was specified as a static modification, and acetylation was allowed as a variable modification on lysine and/or lysine. Furthermore, it should be noted that certain peptides were originally isolated in mouse and later normalized to human sequences as shown by Table 1/FIG. 2.

In proteomics research, it is desirable to validate protein identifications based solely on the observation of a single peptide in one experimental result, in order to indicate that the protein is, in fact, present in a sample. This has led to the development of statistical methods for validating peptide assignments, which are not yet universally accepted, and guidelines for the publication of protein and peptide identification results (see Carr et al., Mol. Cell. Proteomics 3: 531-533 (2004)), which were followed in this Example. However, because the immunoaffinity strategy separates acetylated peptides from unacetylated peptides, observing just one acetylpeptide from a protein is a common result, since many acetylated proteins have only one lysine-acetylated site. For this reason, it is appropriate to use additional criteria to validate acetylpeptide assignments. Assignments are likely to be correct if any of these additional criteria are met: (i) the same sequence is assigned to co-eluting ions with different charge states, since the MS/MS spectrum changes markedly with charge state; (ii) the site is found in more than one peptide sequence context due to sequence overlaps from incomplete proteolysis or use of proteases other than trypsin; (iii) the site is found in more than one peptide sequence context due to homologous but not identical protein isoforms; (iv) the site is found in more than one peptide sequence context due to homologous but not identical proteins among species; and (v) sites validated by MS/MS analysis of synthetic acetylpeptides corresponding to assigned sequences, since the ion trap mass spectrometer produces highly reproducible MS/MS spectra. The last criterion is routinely employed to confirm novel site assignments of particular interest.

All spectra and all sequence assignments made by Sequest were imported into a relational database. The following Sequest scoring thresholds were used to select acetylpeptide assignments that are likely to be correct: RSp<6, XCorr≧2.2, and DeltaCN>0.099. Further, the assigned sequences could be accepted or rejected with respect to accuracy by using the following conservative, two-step process.

In the first step, a subset of high-scoring sequence assignments should be selected by filtering for XCorr values of at least 1.5 for a charge state of +1, 2.2 for +2, and 3.3 for +3, allowing a maximum RSp value of 10. Assignments in this subset should be rejected if any of the following criteria were satisfied: (i) the spectrum contains at least one major peak (at least 10% as intense as the most intense ion in the spectrum) that can not be mapped to the assigned sequence as an a, b, or y ion, as an ion arising from neutral-loss of water or ammonia from a b or y ion, or as a multiply protonated ion; (ii) the spectrum does not contain a series of b or y ions equivalent to at least six uninterrupted residues; or (iii) the sequence is not observed at least five times in all the studies conducted (except for overlapping sequences due to incomplete proteolysis or use of proteases other than trypsin).

In the second step, assignments with below-threshold scores should be accepted if the low-scoring spectrum shows a high degree of similarity to a high-scoring spectrum collected in another study, which simulates a true reference library-searching strategy.

### EXAMPLE 2

### Production of Acetyl-Specific Polyclonal Antibodies for the Detection of Protein Acetylation Signaling Protein Acetylation

Polyclonal antibodies that specifically bind a protein acetylation signal transduction protein only when acetylated at the respective acetylation site disclosed herein (see Table 1/FIG. 2) are produced according to standard methods by first constructing a synthetic peptide antigen comprising the acetylation site sequence and then immunizing an animal to raise antibodies against the antigen, as further described below. Production of exemplary polyclonal antibodies is provided below.

### A. CTTN (Lysine 198).

A 15 amino acid acetyl-peptide antigen, GFGGk*YGIDKDKVDK (where k*=acetyl-lysine) that corresponds to the sequence encompassing the lysine 198 acetylation site in human CTTN transcription Actin binding protein (see Row 18 of Table 1; SEQ ID NO: 17), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals to produce (and subsequently screen) acetyl-specific CTTN (lys198) polyclonal antibodies as described in Immunization/Screening below.

### B. CUL1 (Lysine 689).

A 12 amino acid acetyl-peptide antigen, VNINVPMk*TEQK (where k*=acetyl-lysine) that corresponds to the sequence encompassing the lysine 689 acetylation site in human CULL Cell cycle regulation protein (see Row 82 of Table 1 (SEQ ID NO: 81)), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals to produce (and subsequently screen) acetyl-specific CUL1 (lys689) polyclonal antibodies as described in Immunization/Screening below.

### C. STMN1 (Lysine 119).

A 10 amino acid acetyl-peptide antigen, EAQMAAk*LER (where k*=acetyl-lysine) that corresponds to the sequence encompassing the lysine 119 acetylation site in human STMN1 Cytoskeletal protein (see Row 197 of Table 1 (SEQ ID NO: 196), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals to produce (and subsequently screen) acetyl-specific STMN1 (lys119) antibodies as described in Immunization/Screening below.

### Immunization/Screening.

A synthetic acetyl-peptide antigen as described in A-C above is coupled to KLH, and rabbits are injected intradermally (ID) on the back with antigen in complete Freunds adjuvant (500 μg antigen per rabbit). The rabbits are boosted with same antigen in incomplete Freund adjuvant (250 μg antigen per rabbit) every three weeks. After the fifth boost, bleeds are collected. The sera are purified by Protein A-affinity chromatography by standard methods (see ANTIBODIES: A LABORATORY MANUAL, Cold Spring Harbor, supra.). The eluted immunoglobulins are further loaded onto a non-acetylated synthetic peptide antigen-resin Knotes column to pull out antibodies that bind the non-acetylated form of the acetylation site. The flow through fraction is collected and applied onto an acetyl-synthetic peptide antigen-resin column to isolate antibodies that bind the acetylated form of the site. After washing the column extensively, the bound antibodies (i.e. antibodies that bind a acetylated peptide described in A-C above, but do not bind the non-acetylated form of the peptide) are eluted and kept in antibody storage buffer.

The isolated antibody is then tested for acetyl-specificity using Western blot assay using an appropriate cell line that expresses (or overexpresses) target acetyl-protein (i.e. acetylated CTTN, CULL and STMN1), for example, HeLa, HCT116 and NB-4 respectively. Cells are cultured in DMEM or RPMI supplemented with 10% FBS. Cell are collected, washed with PBS and directly lysed in cell lysis buffer. The protein concentration of cell lysates is then measured. The loading buffer is added into cell lysate and the mixture is boiled at 100° C. for 5 minutes. 20 μl (10 μg protein) of sample is then added onto 7.5% SDS-PAGE gel.

A standard Western blot may be performed according to the Immunoblotting Protocol set out in the CELL SIGNALING TECHNOLOGY,INC. 2003-04 Catalogue, p. 390. The isolated acetyl-specific antibody is used at dilution 1:1000. Acetylation-site specificity of the antibody will be shown by binding of only the acetylated form of the target protein. Isolated acetyl-specific polyclonal antibody does not (substantially) recognize the target protein when not acetylated at the appropriate acetylation site in the non-stimulated cells (e.g. CTTN is not bound when not acetylated at lysine 198).

In order to confirm the specificity of the isolated antibody, different cell lysates containing various acetylated signal transduction proteins other than the target protein are prepared. The Western blot assay is performed again using these cell lysates. The acetyl-specific polyclonal antibody isolated as described above is used (1:1000 dilution) to test reactivity with the different acetylated non-target proteins on Western blot membrane. The acetyl-specific antibody does not significantly cross-react with other acetylated signal transduction proteins, although occasionally slight binding with a highly homologous acetylation-site on another protein may be observed. In such case the antibody may be further purified using affinity chromatography, or the specific immunoreactivity cloned by rabbit hybridoma technology.

### EXAMPLE 3

### Production of Acetyl-Specific Monoclonal Antibodies for the Detection of Protein Acetylation Signaling

Monoclonal antibodies that specifically bind a protein acetylation signal transduction protein only when acetylated at the respective acetylation site disclosed herein (see Table 1/FIG. 2) are produced according to standard methods by first constructing a synthetic peptide antigen comprising the acetylation site sequence and then immunizing an animal to raise antibodies against the antigen, and harvesting spleen cells from such animals to produce fusion hybridomas, as further described below. Production of exemplary monoclonal antibodies is provided below.

### A. MSH2 (Lysine 73).

A 17 amino acid acetyl-peptide antigen, YMGPAGAk*NLQSWLSK (where k*=acetyl-lysine) that corresponds to the sequence encompassing the lysine 73 acetylation site in human MSH2 DNA binding protein (see Row 223 of Table 1 (SEQ ID NO: 222)), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals and harvest spleen cells for generation (and subsequent screening) of acetyl-specific monoclonal MSH2 (lys73) antibodies as described in Immunization/Fusion/Screening below.

### B. PARP1 (Lysine 105).

An 11 amino acid acetyl-peptide antigen GQDGIGSk*AEK (where k*=acetyl-lysine) that corresponds to the sequence encompassing the lysine 105 acetylation site in human PARP1 DNA repair protein (see Row 266 of Table 1 (SEQ ID NO: 265)), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals and harvest spleen cells for generation (and subsequent screening) of acetyl-specific monoclonal PARP1 (lys105) antibodies as described in Immunization/Fusion/Screening below.

### C. RALB (Lysine 179).

A 16 amino acid acetyl-peptide antigen, EIRTk*KMSENLDKNGK (where k*=acetyl-lysine) that corresponds to the sequence encompassing the lysine 179 acetylation site in human RALB G protein/GTPase/Guanine Nucleotide Exchange Factor (see Row 360 of Table 1 (SEQ ID NO: 359)), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals and harvest spleen cells for generation (and subsequent screening) of acetyl-specific monoclonal RALB (lys179) antibodies as described in Immunization/Fusion/Screening below.

### Immunization/Fusion/Screening.

A synthetic acetyl-peptide antigen as described in A-C above is coupled to KLH, and BALB/C mice are injected intradermally (ID) on the back with antigen in complete Freunds adjuvant (e.g. 50 μg antigen per mouse). The mice are boosted with same antigen in incomplete Freund adjuvant (e.g. 25 μg antigen per mouse) every three weeks. After the fifth boost, the animals are sacrificed and spleens are harvested.

Harvested spleen cells are fused to SP2/0 mouse myeloma fusion partner cells according to the standard protocol of Kohler and Milstein (1975). Colonies originating from the fusion are screened by ELISA for reactivity to the acetyl-peptide and non-acetyl-peptide forms of the antigen and by Western blot analysis (as described in Example 1 above). Colonies found to be positive by ELISA to the acetyl-peptide while negative to the non-acetyl-peptide are further characterized by Western blot analysis. Colonies found to be positive by Western blot analysis are subcloned by limited dilution. Mouse ascites are produced from a single clone obtained from subcloning, and tested for acetyl-specificity (against the MSH2, PARP1, or RALB acetyl-peptide antigen, as the case may be) on ELISA. Clones identified as positive on Western blot analysis using cell culture supernatant as having acetyl-specificity, as indicated by a strong band in the induced lane and a weak band in the uninduced lane of the blot, are isolated and subcloned as clones producing monoclonal antibodies with the desired specificity.

Ascites fluid from isolated clones may be further tested by Western blot analysis. The ascites fluid should produce similar results on Western blot analysis as observed previously with the cell culture supernatant, indicating acetyl-specificity against the acetylated target (e.g. RALB acetylated at lysine 179).

### EXAMPLE 4

### Production and Use of AQUA Peptides for the Quantification of Protein Acetylation Signaling Protein

Heavy-isotope labeled peptides (AQUA peptides (internal standards)) for the detection and quantification of a protein acetylation signal transduction protein only when acetylated at the respective acetylation site disclosed herein (see Table 1/FIG. 2) are produced according to the standard AQUA methodology (see Gygi et al., Gerber et al., supra.) methods by first constructing a synthetic peptide standard corresponding to the acetylation site sequence and incorporating a heavy-isotope label. Subsequently, the MSⁿ and LC-SRM signature of the peptide standard is validated, and the AQUA peptide is used to quantify native peptide in a biological sample, such as a digested cell extract. Production and use of exemplary AQUA peptides is provided below.

### A. XRCC5 (Lysine 265).

An AQUA peptide comprising the sequence, IAAYk*SILQER (k*=acetyl-lysine; sequence incorporating ¹⁴C/¹⁵N-labeled leucine (indicated by bold L), which corresponds to the lysine 265 acetylation site in human XRCC5 (see Row 378 in Table 1 (SEQ ID NO: 377)), is constructed according to standard synthesis techniques using, e.g., a Rainin/Protein Technologies, Inc., Symphony peptide synthesizer (see Merrifield, supra.) as further described below in Synthesis & MS/MS Signature. The XRCC5 (lys265) AQUA peptide is then spiked into a biological sample to quantify the amount of acetylated XRCC5 (lys265) in the sample, as further described below in Analysis & Quantification.

### B. GNB2L1 (Lysine 172).

An AQUA peptide comprising the sequence FSPNSSNPIIVSCGWDk*LVK (k*=acetyl-lysine; sequence incorporating ¹⁴C/¹⁵N-labeled leucine (indicated by bold L), which corresponds to the lysine 172 acetylation site in human GNB2 μl Adaptor/Scaffold protein (see Row 36 in Table 1 (SEQ ID NO: 35)), is constructed according to standard synthesis techniques using, e.g., a Rainin/Protein Technologies, Inc., Symphony peptide synthesizer (see Merrifield, supra.) as further described below in Synthesis & MS/MS Signature. The GNB2L1 (lys172) AQUA peptide is then spiked into a biological sample to quantify the amount of acetylated GNB2L1 (lys172) in the sample, as further described below in Analysis & Quantification.

### C. MKI67 (Lysine 379)

An AQUA peptide comprising the sequence, ESVNLGk*SEGFK (K*=acetyllysine; sequence incorporating ¹⁴C/¹⁵N-labeled phenylalanine (indicated by bold F), which corresponds to the lysine 379 acetylation site in human MKI67 Cell cycle regulation protein (see Row 88 in Table 1 (SEQ ID NO: 87)), is constructed according to standard synthesis techniques using, e.g., a Rainin/Protein Technologies, Inc., Symphony peptide synthesizer (see Merrifield, supra.) as further described below in Synthesis & MS/MS Signature. The MKI67 (lys379) AQUA peptide is then spiked into a biological sample to quantify the amount of acetylated MKI67 (lys379) in the sample, as further described below in Analysis & Quantification.

### D. MAPT (Lysine 311).

An AQUA peptide comprising the sequence, VQIVYk*PVDLSK (k*=acetyl-lysine; sequence incorporating ¹⁴C/¹⁵N-labeled proline (indicated by bold P), which corresponds to the lysine 311 acetylation site in human MAPT Cytoskeletal protein (see Row 189 in Table 1 (SEQ ID NO: 188)), is constructed according to standard synthesis techniques using, e.g., a Rainin/Protein Technologies, Inc., Symphony peptide synthesizer (see Merrifield, supra.) as further described below in Synthesis & MS/MS Signature. The MAPT (lys311) AQUA peptide is then spiked into a biological sample to quantify the amount of acetylated MAPT (lys311) in the sample, as further described below in Analysis & Quantification.

### Synthesis & MS/MS Spectra.

Fluorenylmethoxycarbonyl (Fmoc)-derivatized amino acid monomers may be obtained from AnaSpec (San Jose, Calif.). Fmoc-derivatized stable-isotope monomers containing one ¹⁵N and five to nine ¹³C atoms may be obtained from Cambridge Isotope Laboratories (Andover, Mass.). Preloaded Wang resins may be obtained from Applied Biosystems. Synthesis scales may vary from 5 to 25 μmol. Amino acids are activated in situ with 1-H-benzotriazolium, 1-bis(dimethylamino) methylene]-hexafluorophosphate (1-),3-oxide:1-hydroxybenzotriazole hydrate and coupled at a 5-fold molar excess over peptide. Each coupling cycle is followed by capping with acetic anhydride to avoid accumulation of one-residue deletion peptide by-products. After synthesis peptide-resins are treated with a standard scavenger-containing trifluoroacetic acid (TFA)-water cleavage solution, and the peptides are precipitated by addition to cold ether. Peptides (i.e. a desired AQUA peptide described in A-D above) are purified by reversed-phase C18 HPLC using standard TFA/acetonitrile gradients and characterized by matrix-assisted laser desorption ionization-time of flight (Biflex III, Bruker Daltonics, Billerica, Mass.) and ion-trap (ThermoFinnigan, LCQ DecaXP) MS.

MS/MS spectra for each AQUA peptide should exhibit a strong y-type ion peak as the most intense fragment ion that is suitable for use in an SRM monitoring/analysis. Reverse-phase microcapillary columns (0.1 Å˜150-220 mm) are prepared according to standard methods. An Agilent 1100 liquid chromatograph may be used to develop and deliver a solvent gradient [0.4% acetic acid/0.005% heptafluorobutyric acid (HFBA)/7% methanol and 0.4% acetic acid/0.005% HFBA/65% methanol/35% acetonitrile] to the microcapillary column by means of a flow splitter. Samples are then directly loaded onto the microcapillary column by using a FAMOS inert capillary autosampler (LC Packings, San Francisco) after the flow split. Peptides are reconstituted in 6% acetic acid/0.01% TFA before injection.

### Analysis & Quantification.

Target protein (e.g. a acetylated protein of A-D above) in a biological sample is quantified using a validated AQUA peptide (as described above). The IAP method is then applied to the complex mixture of peptides derived from proteolytic cleavage of crude cell extracts to which the AQUA peptides have been spiked in.

LC-SRM of the entire sample is then carried out. MS/MS may be performed by using a ThermoFinnigan (San Jose, Calif.) mass spectrometer (LCQ DecaXP ion trap or TSQ Quantum triple quadrupole). On the DecaXP, parent ions are isolated at 1.6 m/z width, the ion injection time being limited to 150 ms per microscan, with two microscans per peptide averaged, and with an AGC setting of 1×10⁸; on the Quantum, Q1 is kept at 0.4 and Q3 at 0.8 m/z with a scan time of 200 ms per peptide. On both instruments, analyte and internal standard are analyzed in alternation within a previously known reverse-phase retention window; well-resolved pairs of internal standard and analyte are analyzed in separate retention segments to improve duty cycle. Data are processed by integrating the appropriate peaks in an extracted ion chromatogram (60.15 m/z from the fragment monitored) for the native and internal standard, followed by calculation of the ratio of peak areas multiplied by the absolute amount of internal standard (e.g., 500 fmol).

What is claimed is:

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. An isolated acetylation site-specific antibody that specifically binds a human acetylation signaling protein selected from Column A of Table 1, Rows 294, 355, 317, 90 and 398 only when acetylated at the lysine listed in corresponding Column D of Table 1, comprised within the acetylatable peptide sequence listed in corresponding Column E of Table 1 (SEQ ID NOs: 293, 354, 316, 89 and 397), wherein said antibody does not bind said signaling protein when not acetylated at said lysine.

50. An isolated acetylation site-specific antibody that specifically binds a human acetylation signaling protein selected from Column A of Table 1, Rows 294, 355, 317, 90 and 398 only when not acetylated at the lysine listed in corresponding Column D of Table 1, comprised within the acetylatable peptide sequence listed in corresponding Column E of Table 1 (SEQ ID NOs: 293, 354, 316, 89 and 397), wherein said antibody does not bind said signaling protein when acetylated at said lysine.

51. A method selected from the group consisting of:
(a) a method for detecting a human acetylation signaling protein selected from Column A of Table 1, Rows 294, 355, 317, 90 and 398 wherein said human acetylation signaling protein is acetylated at the lysine listed in corresponding Column D of Table 1, comprised within the acetylatable peptide sequence listed in corresponding Column E of Table 1 (SEQ ID NOs: 293, 354, 316, 89 and 397), comprising the step of adding an isolated acetylation-specific antibody according to claim 49, to a sample comprising said human acetylation signaling protein under conditions that permit the binding of said antibody to said human acetylation signaling protein, and detecting bound antibody;
(b) a method for quantifying the amount of a human acetylation signaling protein listed in Column A of Table 1, Rows 294, 355, 317, 90 and 398 that is acetylated at the corresponding lysine listed in Column D of Table 1, comprised within the acetylatable peptide sequence listed in corresponding Column E of Table 1 (SEQ ID NOs: 293, 354, 316, 89 and 397), in a sample using a heavy-isotope labeled peptide (AQUA™ peptide), said labeled peptide comprising a acetylated lysine at said corresponding lysine listed Column D of Table 1, comprised within the acetylatable peptide sequence listed in corresponding Column E of Table 1 as an internal standard; and
(c) a method comprising step (a) followed by step (b).

52. The method of claim 51, wherein said isolated acetylation-specific antibody is capable of specifically binding ALDOA only when acetylated at ALDOA, comprised within the acetylatable peptide sequence listed in Column E, Row 294, of Table 1 (SEQ ID NO: 293), wherein said antibody does not bind said protein when not acetylated at said lysine.

53. The method of claim 51, wherein said isolated acetylation-specific antibody is capable of specifically binding ALDOA only when not acetylated at ALDOA, comprised within the acetylatable peptide sequence listed in Column E, Row 294, of Table 1 (SEQ ID NO: 293), wherein said antibody does not bind said protein when acetylated at said lysine.

54. The method of claim 51, wherein said isolated acetylation-specific antibody is capable of specifically binding RAN only when acetylated at K71, comprised within the acetylatable peptide sequence listed in Column E, Row 355, of Table 1 (SEQ ID NO: 354), wherein said antibody does not bind said protein when not acetylated at said lysine.

55. The method of claim 51, wherein said isolated acetylation-specific antibody is capable of specifically binding RAN only when not acetylated at K71, comprised within the acetylatable peptide sequence listed in Column E, Row 355, of Table 1 (SEQ ID NO: 354), wherein said antibody does not bind said protein when acetylated at said lysine.

56. The method of claim 51, wherein said isolated acetylation-specific antibody is capable of specifically binding PKLR only when acetylated at K305, comprised within the acetylatable peptide sequence listed in Column E, Row 317, of Table 1 (SEQ ID NO: 316), wherein said antibody does not bind said protein when not acetylated at said lysine.

57. The method of claim 51, wherein said isolated acetylation-specific antibody is capable of specifically binding PKLR only when not acetylated at K305, comprised within the acetylatable peptide sequence listed in Column E, Row 317, of Table 1 (SEQ ID NO: 316), wherein said antibody does not bind said protein when acetylated at said lysine.

58. The method of claim 51, wherein said isolated acetylation-specific antibody is capable of specifically binding PCNA only when acetylated at K80, comprised within the acetylatable peptide sequence listed in Column E, Row 90, of Table 1 (SEQ ID NO: 89), wherein said antibody does not bind said protein when not acetylated at said lysine.

59. The method of claim 51, wherein said isolated acetylation-specific antibody is capable of specifically binding PCNA only when not acetylated at K80, comprised within the acetylatable peptide sequence listed in Column E, Row 90, of Table 1 (SEQ ID NO: 89), wherein said antibody does not bind said protein when acetylated at said lysine.

60. The method of claim 51, wherein said isolated acetylation-specific antibody is capable of specifically binding PPIA only when acetylated at K82, comprised within the acetylatable peptide sequence listed in Column E, Row 398, of Table 1 (SEQ ID NO: 397), wherein said antibody does not bind said protein when not acetylated at said lysine.

61. The method of claim 51, wherein said isolated acetylation-specific antibody is capable of specifically binding PPIA only when not acetylated at K82, comprised within the acetylatable peptide sequence listed in Column E, Row 398, of Table 1 (SEQ ID NO: 397), wherein said antibody does not bind said protein when acetylated at said lysine.
